(19) United States
(12) Patent Application Publication
(10) Pub. No.: US 2021/0094973 A1
(43) Pub. Date: Apr. 1, 2021

(54) HETEROCYCLIC COMPOUNDS

(71) Applicant: Hoffmann-La Roche Inc., Little Falls, NJ (US)

(72) Inventors: Luca GOBBI, Basel (CH); Uwe GRETHER, Basel (CH); Katrin GROEBKE ZBINDEN, Basel (CH); Benoit HORNSPERGER, Basel (CH); Carsten KROLL, Basel (CH); Bernd KUHN, Basel (CH); Marius Daniel Rinaldo LUTZ, Basel (CH); Fionn O'HARA, Basel (CH); Hans RICHTER, Basel (CH); Martin RITTER, Basel (CH)

(73) Assignee: Hoffmann-La Roche Inc., Little Falls, NJ (US)

(21) Appl. No.: 17/027,976

(22) Filed: Sep. 22, 2020

(30) Foreign Application Priority Data
Sep. 24, 2019 (EP) 19199108.2

(51) Int. Cl. C07D 498/04; A61K 9/20; A61K 9/48

(52) U.S. Cl. CPC C07D 498/04; A61K 9/2009; A61K 9/2054; A61K 9/485; A61K 9/4866; A61K 9/4858; A61K 9/2059

(57) ABSTRACT

The invention provides new heterocyclic compounds having the general formula (I)

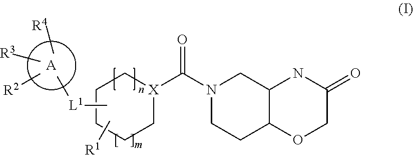

wherein A, L¹, X, m, n and R¹ to R⁴ are as described herein, compositions including the compounds, processes of manufacturing the compounds and methods of using the compounds.

### CROSS-REFERENCE TO RELATED APPLICATIONS

This application claims priority to EP Application No. 19199108.2, filed Sep. 24, 2019, the disclosure of which is incorporated herein by reference in its entirety.

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA2-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process.

The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-a) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403). Importantly, genetic and/or pharmacological disruption of MAGL activity also blocks LPS-induced activation of microglial cells in the brain (Nomura, D. K., et al., Science 2011, 334, 809).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J. Pharmacol. Exp. Ther. 2015, 353, 424) and on mental disorders, such as depression in chronic stress models (Zhong P. et al., Neuropsychopharmacology 2014, 39, 1763).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bemal-Chico, A., et al., Glia 2015, 63, 163). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421). Inhibition of MAGL was also shown to promote remyelination and functional recovery in a mouse model of progressive multiple sclerosis (Feliu A. et al., Journal of Neuroscience 2017, 37 (35), 8385).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez L. et al., PLoS One 2009, 4(9), e6893). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright K. et al., Gastroenterology 2005, 129(2), 437-453; Duncan, M. et al., Aliment Pharmacol Ther 2005, 22(8), 667-683). Activation of CB1 produces anti-emetic, anti-motility, and anti-inflammatory effect, and help to modulate pain (Perisetti, A. et al., Ann Gastroenterol 2020, 33(2), 134-144). CB2 receptors are expressed in immune cells such as plasma cells and macrophages, in the lamina propria of the GI tract (Wright K. et al., Gastroenterology 2005, 129(2), 437-453), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez L. et al., PLoS One 2009, 4(9), e6893) and 2-AG levels are increased in plasma of IBD patients (Grill, M. et al., Sci Rep 2019, 9(1), 2358). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez L. et al., PLoS One 2009, 4(9), e6893). Furthermore, MAGL inhibition improves gut wall integrity and intestinal permeability via a CB1 driven MoA (Wang, J. et al., Biochem Biophys Res Commun 2020, 525(4), 962-967).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides new heterocyclic compounds having the general formula (I)

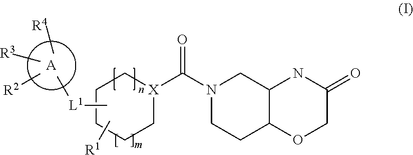

wherein A, L¹, X, m, n and R¹ to R⁴ are as defined herein.

In a further aspect, the present invention provides a process of manufacturing the compounds of formula (I) as described herein, comprising:
(a) reacting a first amine 4a,5,6,7,8,8a-hexahydro-4H-pyrido[4,3-b][1,4]oxazin-3-one (1),

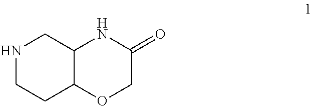 [SMILES: O=C1COC2CCNCC2N1]

with a second amine 2, wherein A, L¹, m, n, and R¹ to R⁴ are as defined herein

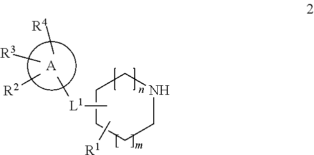

in the presence of a base and a urea forming reagent, to form a compound of formula (IA), wherein A, L¹, m, n, and R¹ to R⁴ are as defined herein,

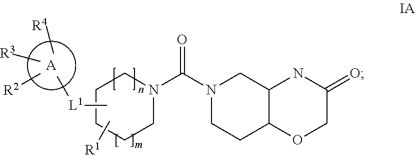

or
(b) reacting 4a,5,6,7,8,8a-hexahydro-4H-pyrido[4,3-b][1,4]oxazin-3-one (1),

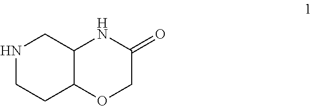 [SMILES: O=C1COC2CCNCC2N1]

with a carboxylic acid 3a, wherein A, L¹, m, n, and R¹ to R⁴ are as defined herein,

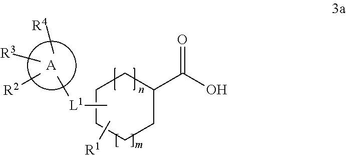

in the presence of a coupling reagent, such as CDI, DCC, HATU, HBTU, HOBT, TBTU, T3P or Mukaiyama reagent and optionally a base, such as TEA, DIPEA or DMAP, to form a compound of formula (IB), wherein A, L¹, m, n, and R¹ to R⁴ are as defined herein,

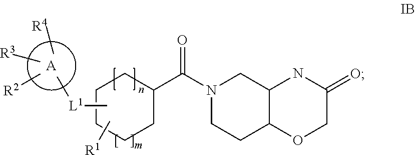

or
(c) reacting 4a,5,6,7,8,8a-hexahydro-4H-pyrido[4,3-b][1,4]oxazin-3-one (1),

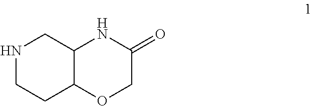 [SMILES: O=C1COC2CCNCC2N1]

with a carboxylic acid chloride 3b, wherein A, L¹, m, n, and R¹ to R⁴ are as defined herein,

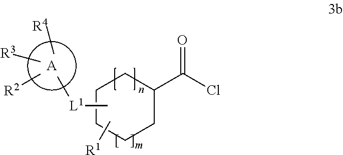

in the presence of a base, e.g. TEA, Huenig's base, pyridine, DMAP or lithium bis(trimethylsilyl)amide, to form a compound of formula (IB), wherein A, L¹, m, n, and R¹ to R⁴ are as defined herein,

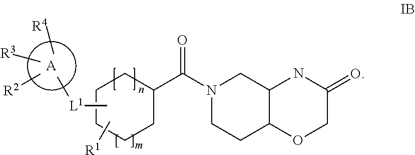

In a further aspect, the present invention provides a compound of formula (I) as described herein, when manufactured according to the processes described herein.

In a further aspect, the present invention provides a compound of formula (I) as described herein, for use as therapeutically active substance.

In a further aspect, the present invention provides a pharmaceutical composition comprising a compound of formula (I) as described herein and a therapeutically inert carrier.

In a further aspect, the present invention provides the use of a compound of formula (I) as described herein for inhibiting monoacylglycerol lipase (MAGL) in a mammal.

In a further aspect, the present invention provides the use of a compound of formula (I) as described herein, or a pharmaceutically acceptable salt thereof, or of a pharmaceutical composition described herein for the treatment or prophylaxis of neuroinflammation, neurodegenerative diseases, pain, cancer, mental disorders and/or inflammatory bowel disease in a mammal.

In a further aspect, the present invention provides the use of a compound of formula (I) as described herein, or a pharmaceutically acceptable salt thereof, or of a pharmaceutical composition described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms, e.g., 1, 2, 3, 4, 5, or 6 carbon atoms (“C₁-C₆-alkyl”). In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl.

A particularly preferred, yet non-limiting example of alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁-C₆-alkyl”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl and cycloheptyl. Particularly preferred, yet non-limiting examples of cycloalkyl include cyclopropyl, cyclobutyl, and cyclopentyl.

The term “heterocyclyl” as used herein refers to a saturated or partly unsaturated mono- or bicyclic, preferably monocyclic ring system of 3 to 10 ring atoms, preferably 3 to 8 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of monocyclic heterocyclyl groups include azetidin-3-yl, azetidin-2-yl, oxetan-3-yl, oxetan-2-yl, 2-oxopyrrolidin-1-yl, 2-oxopyrrolidin-3-yl, 5-oxopyrrolidin-2-yl, 5-oxopyrrolidin-3-yl, 2-oxo-1-piperidyl, 2-oxo-3-piperidyl, 2-oxo-4-piperidyl, 6-oxo-2-piperidyl, 6-oxo-3-piperidyl, 1-piperidinyl, 2-piperidinyl, 3-piperidinyl, 4-piperidinyl, morpholino, morpholin-2-yl, morpholin-3-yl, tetrahydropyranyl, and tetrahydrofuranyl. Particularly preferred, yet non-limiting examples of heterocyclyl groups include tetrahydropyranyl, and tetrahydrofuranyl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic, bicyclic or tricyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include thiadiazolyl, imidazolyl, oxadiazolyl, 1H-indazoyl, pyrazolyl, pyridyl, and pyridazinyl.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl and 2,2,2-trifluoro-1,1-dimethyl-ethoxy.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid.

Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The term “urea forming reagent” refers to a chemical compound that is able to render a first amine to a species that will react with a second amine, thereby forming an urea derivative. Non-limiting examples of a urea forming reagent include bis(trichloromethyl) carbonate, phosgene, trichloromethyl chloroformate, (4-nitrophenyl)carbonate and 1,1′-carbonyldiimidazole. The urea forming reagents described in G. Sartori et al., Green Chemistry 2000, 2, 140 are incorporated herein by reference.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician.

However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and psychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

Compounds of the Invention

In a first aspect (A), the present invention provides compounds of Formula (I)

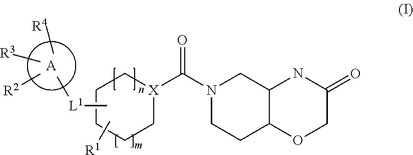

or a pharmaceutically acceptable salts thereof, wherein:
R¹ is hydrogen or C₁-C₆-alkyl;
R², R³, and R⁴ are independently selected from hydrogen, a group

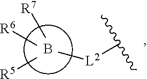

C₁-C₆-alkyl, C₁-C₆-alkoxy, halo-C₁-C₆-alkyl, halo-C₁-C₆-alkoxy, halogen, and SF₅;
R⁵, R⁶, and R⁷ are independently selected from hydrogen, halogen, and halo-C₁-C₆-alkyl;
X is CH or N;
m and n are both 1; or
m and n are both 0;
A is selected from C₆-C₁₄-aryl and 5-14 membered heteroaryl;
L¹ is selected from a covalent bond, —CH₂—, —OCHR^{L}—, —CHR^{L}O—, and —NHC(O)—;
R^{L} is selected from hydrogen and C₁-C₆-alkyl; and
B is
   (i) C₆-C₁₄-aryl and L² is a covalent bond; or
   (ii) 3-14 membered heterocyclyl or C₃-C₁₀-cycloalkyl; and L² is selected from a covalent bond, —O—, and —CH₂O—.

The invention also provides the following enumerated Embodiments (E) of the first aspect (A1) of the invention:
E1. The compound of formula (I) according to A1, or a pharmaceutically acceptable salt thereof, wherein the compound of formula (I) is not selected from:
rac-cis-6-(4-(5-Chloro-1-methyl-1H-indol-3-yl)piperidine-1-carbonyl)hexahydro-2H-pyrido[4,3-b][1,4]oxazin-3(4H)-one;
rac-cis-6-(4-(9H-Fluoren-9-yl)piperidine-1-carbonyl)hexahydro-2H-pyrido[4,3-b][1,4]oxazin-3(4H)-one;
(+)-cis-6-[4-(6-Fluoro-1H-indol-3-yl)piperidine-1-carbonyl]-4,4a,5,7,8,8a-hexahydropyrido[4,3-b][1,4]oxazin-3-one;
rac-cis-6-[4-(6-Fluoro-1H-indol-3-yl)piperidine-1-carbonyl]-4,4a,5,7,8,8a-hexahydropyrido[4,3-b][1,4]oxazin-3-one;
rac-cis-6-(4-(5-Fluorobenzo[d]isoxazol-3-yl)piperidine-1-carbonyl)hexahydro-2H-pyrido[4,3-b][1,4]oxazin-3(4H)-one;
rac-cis-6-(4-(5-Chloro-1H-indol-3-yl)piperazine-1-carbonyl)hexahydro-2H-pyrido[4,3-b][1,4]oxazin-3(4H)-one;
rac-cis-6-(4-(1-Methyl-1H-indazol-5-yl)piperidine-1-carbonyl)hexahydro-2H-pyrido[4,3-b][1,4]oxazin-3(4H)-one;
(+)- or (−)-cis-6-(4-(5-Chloro-1-cyclopropyl-1H-indol-3-yl)piperidine-1-carbonyl)hexahydro-2H-pyrido[4,3-b][1,4]oxazin-3(4H)-one;
(+)- or (−)-cis-6-(4-(5-Chloro-1-(oxetan-3-yl)-1H-indol-3-yl)piperidine-1-carbonyl)hexahydro-2H-pyrido[4,3-b][1,4]oxazin-3(4H)-one;
rac-cis-6-(4-(1-(2-Chloro-4-fluorophenoxy)ethyl)piperidine-1-carbonyl)hexahydro-2H-pyrido[4,3-b][1,4]oxazin-3(4H)-one;
rac-cis-6-(4-(5-(Trifluoromethyl)pyridin-3-yl)piperidine-1-carbonyl)hexahydro-2H-pyrido[4,3-b][1,4]oxazin-3(4H)-one;
(+) or (−)-cis-6-(4-((S or R)-1-(2-chloro-4-fluorophenoxy)ethyl)piperidine-1-carbonyl)hexahydro-2H-pyrido[4,3-b][1,4]oxazin-3(4H)-one;
(+) or (−)-cis-6-(4-((R or S)-1-(2-chloro-4-fluorophenoxy)ethyl)piperidine-1-carbonyl)hexahydro-2H-pyrido[4,3-b][1,4]oxazin-3(4H)-one;
(+) or (−)-cis-6-(4-(5-Methoxypyridin-3-yl)piperidine-1-carbonyl)hexahydro-2H-pyrido[4,3-b][1,4]oxazin-3(4H)-one;
(+) or (−)-cis-6-(4-(5-(Trifluoromethoxy)pyridin-2-yl)piperidine-1-carbonyl)hexahydro-2H-pyrido[4,3-b][1,4]oxazin-3(4H)-one;
(+) or (−)-cis-6-(4-(5-Ethylpyridin-3-yl)piperidine-1-carbonyl)hexahydro-2H-pyrido[4,3-b][1,4]oxazin-3(4H)-one;
(+) or (−)-cis-6-(4-(5-(1,1-Difluoroethyl)pyridin-2-yl)piperidine-1-carbonyl)hexahydro-2H-pyrido[4,3-b][1,4]oxazin-3(4H)-one;
(+) or (−)-cis-6-(4-(6-Chloro-1-methyl-H-indazol-3-yl)piperidine-1-carbonyl)hexahydro-2H-pyrido[4,3-b][1,4]oxazin-3(4H)-one;
(4aR,8aS)-6-((3R)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-((3S)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-((4S)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-(4-(2-Cyclopropylpyridin-4-yl)piperidine-1-carbonyl)hexahydro-2H-pyrido[4,3-b][1,4]oxazin-3(4H)-one;
(4aR,8aS)-6-(3-(1-(2-Chloro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-(1-(2-Chloro-4-(trifluoromethyl)phenoxy)ethyl)azetidine-1-carbonyl)hexahydro-2H-pyrido[4,3-b][1,4]oxazin-3(4H)-one;
(+) or (−)-(4aR,8aS)-6-[3-[1-[4-(Trifluoromethyl)phenyl]ethoxy]azetidine-1-carbonyl]-4,4a,5,7,8,8a-hexahydropyrido[4,3-b][1,4]oxazin-3-one;
(4aR,8aS)-6-(3-(1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-(1-(4-(Trifluoromethyl)phenoxy)ethyl)azetidine-1-carbonyl)hexahydro-2H-pyrido[4,3-b][1,4]oxazin-3(4H)-one;
(4aR,8aS)-6-[4-[1-[4-(Trifluoromethyl)phenyl]ethoxy]piperidine-1-carbonyl]-4,4a,5,7,8,8a-hexahydropyrido[4,3-b][1,4]oxazin-3-one;
(4aR,8aS)-6-(3-((S)-1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-((R)-1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-((S)-1-(4-(Trifluoromethyl)phenoxy)ethyl)azetidine-1-carbonyl)hexahydro-2H-pyrido[4,3-b][1,4]oxazin-3(4H)-one;
(4aR,8aS)-6-(3-((R)-1-(4-(trifluoromethyl)phenoxy)ethyl)azetidine-1-carbonyl)hexahydro-2H-pyrido[4,3-b][1,4]oxazin-3(4H)-one;
(4aR,8aS)-6-(4-(5-Methyl-6-(trifluoromethyl)pyridin-3-yl)piperidine-1-carbonyl)hexahydro-2H-pyrido[4,3-b][1,4]oxazin-3(4H)-one;
(4aR,8aS)-6-(4-(5,6,7,8-Tetrahydroquinolin-4-yl)piperidine-1-carbonyl)hexahydro-2H-pyrido[4,3-b][1,4]oxazin-3(4H)-one;
(4aR,8aS)-6-(3-(4-Bromophenyl)azetidine-1-carbonyl)hexahydro-2H-pyrido[4,3-b][1,4]oxazin-3(4H)-one;
(4aR,8aS)-6-(3-(4′-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(2′-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(2′,4′-Dichloro-[1,1′-biphenyl]-4-yl)azetidine-1-carbonyl)hexahydro-2H-pyrido[4,3-b][1,4]oxazin-3(4H)-one;
(4aR,8aS)-6-(3-(1-(4-(Trifluoromethyl)phenyl)ethoxy)azetidine-1-carbonyl)hexahydro-2H-pyrido[4,3-b][1,4]oxazin-3(4H)-one;
(4aR,8aS)-6-(3-(1-(4-(Trifluoromethyl)phenyl)ethoxy)azetidine-1-carbonyl)hexahydro-2H-pyrido[4,3-b][1,4]oxazin-3(4H)-one;
(4aR,8aS)-6-[3-(S or R)-[1-(2-Chloro-4-fluoro-phenyl)ethoxy]azetidine-1-carbonyl]-4,4a,5,7,8,8a-hexahydropyrido[4,3-b][1,4]oxazin-3-one;
(4aR,8aS)-6-[3-(R or S)-[1-(2-Chloro-4-fluoro-phenyl)ethoxy]azetidine-1-carbonyl]-4,4a,5,7,8,8a-hexahydropyrido[4,3-b][1,4]oxazin-3-one;
(4aR,8aS)-6-[3-[4-(Trifluoromethoxy)phenyl]azetidine-1-carbonyl]-4,4a,5,7,8,8a-hexahydropyrido[4,3-b][1,4]oxazin-3-one;
(4aR,8aS)-6-(3-(4-Bromophenyl)-3-fluoroazetidine-1-carbonyl)hexahydro-2H-pyrido[4,3-b][1,4]oxazin-3(4H)-one;
(4aR,8aS)-6-(3-(4-Bromophenyl)-3-hydroxyazetidine-1-carbonyl)hexahydro-2H-pyrido[4,3-b][1,4]oxazin-3(4H)-one;
(4aR,8aS)-6-(3-(4-Bromophenyl)-3-methylazetidine-1-carbonyl)hexahydro-2H-pyrido[4,3-b][1,4]oxazin-3(4H)-one;
(4aR,8aS)-6-(3-(2′-(Trifluoromethyl)-[1,1′-biphenyl]-4-yl)azetidine-1-carbonyl)hexahydro-2H-pyrido[4,3-b][1,4]oxazin-3(4H)-one;
(4aR,8aS)-6-(3-(2′,4′-Difluoro-[1,1′-biphenyl]-4-yl)azetidine-1-carbonyl)hexahydro-2H-pyrido[4,3-b][1,4]oxazin-3(4H)-one;
(4aR,8aS)-6-(3-(4-(3-(Trifluoromethyl)azetidin-1-yl)phenyl)azetidine-1-carbonyl)hexahydro-2H-pyrido[4,3-b][1,4]oxazin-3(4H)-one;
(4aR,8aS)-6-(3-(2-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(4-Bromo-3-chlorophenyl)azetidine-1-carbonyl)hexahydro-2H-pyrido[4,3-b][1,4]oxazin-3(4H)-one;
(4aR,8aS)-6-[3-[4-(4-Chloro-2-fluoro-phenyl)phenyl]azetidine-1-carbonyl]-4,4a,5,7,8,8a-hexahydropyrido[4,3-b][1,4]oxazin-3-one;
(4aR,8aS)-6-[3-[4-(2-Chloro-4-fluoro-phenyl)phenyl]azetidine-1-carbonyl]-4,4a,5,7,8,8a-hexahydropyrido[4,3-b][1,4]oxazin-3-one;
(4aR,8aS)-6-(3-(3-Bromophenyl)azetidine-1-carbonyl)hexahydro-2H-pyrido[4,3-b][1,4]oxazin-3(4H)-one;
(4aR,8aS)-6-(3-(4-(tert-Butyl)phenyl)azetidine-1-carbonyl)hexahydro-2H-pyrido[4,3-b][1,4]oxazin-3(4H)-one;
(4aR,8aS)-6-[3-(4-Phenylphenyl)azetidine-1-carbonyl]-4,4a,5,7,8,8a-hexahydropyrido[4,3-b][1,4]oxazin-3-one;
(4aR,8aS)-6-[3-[4-[2-(Difluoromethyl)phenyl]phenyl]azetidine-1-carbonyl]-4,4a,5,7,8,8a-hexahydropyrido[4,3-b][1,4]oxazin-3-one;
(4aR,8aS)-6-(3-(6-Chloropyridin-3-yl)azetidine-1-carbonyl)hexahydro-2H-pyrido[4,3-b][1,4]oxazin-3(4H)-one;
(4aR,8aS)-6-(3-(4-(Trifluoromethyl)phenyl)azetidine-1-carbonyl)hexahydro-2H-pyrido[4,3-b][1,4]oxazin-3(4H)-one;
(4aR,8aS)-6-(3-(4-(1,1-Difluoroethyl)phenyl)azetidine-1-carbonyl)hexahydro-2H-pyrido[4,3-b][1,4]oxazin-3(4H)-one;
(4aR,8aS)-6-(3-(6-(2,4-Dichlorophenyl)pyridin-3-yl)azetidine-1-carbonyl)hexahydro-2H-pyrido[4,3-b][1,4]oxazin-3(4H)-one;
(4aR,8aS)-6-(3-(4-(3,3-Difluoroazetidin-1-yl)phenyl)azetidine-1-carbonyl)hexahydro-2H-pyrido[4,3-b][1,4]oxazin-3(4H)-one;
(4aR,8aS)-6-(4-(4-Bromophenyl)piperidine-1-carbonyl)hexahydro-2H-pyrido[4,3-b][1,4]oxazin-3(4H)-one;
(4aR,8aS)-6-[3-[4-(2,2,2-Trifluoroethoxy)phenyl]azetidine-1-carbonyl]-4,4a,5,7,8,8a-hexahydropyrido[4,3-b][1,4]oxazin-3-one;
(4aR,8aS)-6-(3-(4-(2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-(3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(3-Bromophenyl)pyrrolidine-1-carbonyl)hexahydro-2H-pyrido[4,3-b][1,4]oxazin-3(4H)-one;
(4aR,8aS)-6-(4-(4-Bromophenyl)piperazine-1-carbonyl)hexahydro-2H-pyrido[4,3-b][1,4]oxazin-3(4H)-one;
(4aR,8aS)-6-(4-(2′,4′-dichloro-[1,1′-biphenyl]-4-yl)piperidine-1-carbonyl)hexahydro-2H-pyrido[4,3-b][1,4]oxazin-3(4H)-one;
(4aR,8aS)-6-(3-(4-(3-azabicyclo[3.1.0]hexan-3-yl)phenyl)azetidine-1-carbonyl)hexahydro-2H-pyrido[4,3-b][1,4]oxazin-3(4H)-one;
(4aR,8aS)-6-(3-(3-(trifluoromethoxy)phenyl)azetidine-1-carbonyl)hexahydro-2H-pyrido[4,3-b][1,4]oxazin-3(4H)-one;
(4aR,8aS)-6-(3-(6-(2-(trifluoromethyl)pyrrolidin-1-yl)pyridin-3-yl)azetidine-1-carbonyl)hexahydro-2H-pyrido[4,3-b][1,4]oxazin-3(4H)-one;
(4aR,8aS)-6-(3-(4-(5-Azaspiro[2.4]heptan-5-yl)phenyl)azetidine-1-carbonyl)hexahydro-2H-pyrido[4,3-b][1,4]oxazin-3(4H)-one;
(4aR,8aS)-6-(3-(4-(Pentafluoro-16-sulfaneyl)phenyl)azetidine-1-carbonyl)hexahydro-2H-pyrido[4,3-b][1,4]oxazin-3(4H)-one;
(4aR,8aS)-6-(3-(5-Chloropyridin-2-yl)azetidine-1-carbonyl)hexahydro-2H-pyrido[4,3-b][1,4]oxazin-3(4H)-one;
(4aR,8aS)-6-(3-(2-Fluoro-4-(trifluoromethoxy)phenyl)azetidine-1-carbonyl)hexahydro-2H-pyrido[4,3-b][1,4]oxazin-3(4H)-one;
(4aR,8aS)-6-[3-(6-Methoxypyridin-3-yl)azetidine-1-carbonyl]-4,4a,5,7,8,8a-hexahydropyrido[4,3-b][1,4]oxazin-3-one;
(4aR,8aS)-6-(3-(4-Bromophenyl)pyrrolidine-1-carbonyl)hexahydro-2H-pyrido[4,3-b][1,4]oxazin-3(4H)-one;
(4aR,8aS)-6-(3-Phenylazetidine-1-carbonyl)hexahydro-2H-pyrido[4,3-b][1,4]oxazin-3(4H)-one;
(4aR,8aS)-6-(4-Phenylpiperidine-1-carbonyl)hexahydro-2H-pyrido[4,3-b][1,4]oxazin-3(4H)-one;
(4aR,8aS)-6-[3-[4-(2,2,2-Trifluoroethyl)phenyl]azetidine-1-carbonyl]-4,4a,5,7,8,8a-hexahydropyrido[4,3-b][1,4]oxazin-3-one;
(4aR,8aS)-6-[3-[4-[1-(Trifluoromethyl)cyclopropyl]phenyl]azetidine-1-carbonyl]-4,4a,5,7,8,8a-hexahydropyrido[4,3-b][1,4]oxazin-3-one;
(4aR,8aS)-6-[3-[4-(6,6-Difluoro-2-azaspiro[3.3]heptan-2-yl)phenyl]azetidine-1-carbonyl]-4,4a,5,7,8,8a-hexahydropyrido[4,3-b][1,4]oxazin-3-one;
(4aR,8aS)-6-(3-(4-(8-Oxa-3-azabicyclo[3.2.1]octan-3-yl)phenyl)azetidine-1-carbonyl)hexahydro-2H-pyrido[4,3-b][1,4]oxazin-3(4H)-one;
(4aR,8aS)-6-(3-(5-(2,4-Dichlorophenyl)pyridin-2-yl)azetidine-1-carbonyl)hexahydro-2H-pyrido[4,3-b][1,4]oxazin-3(4H)-one;
(4aR,8aS)-6-(3-(4-((S)-2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-((R)-2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-[3-[4-(1-Piperidyl)phenyl]azetidine-1-carbonyl]-4,4a,5,7,8,8a-hexahydropyrido[4,3-b][1,4]oxazin-3-one;
(4aR,8aS)-6-(3-(4-((R or S)-3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-((S or R)-3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-(3-Fluoroazetidin-1-yl)phenyl)azetidine-1-carbonyl)hexahydro-2H-pyrido[4,3-b][1,4]oxazin-3(4H)-one;
(4aR,8aS)-6-(3-(3-Fluoro-4-(trifluoromethoxy)phenyl)azetidine-1-carbonyl)hexahydro-2H-pyrido[4,3-b][1,4]oxazin-3(4H)-one;
(4aR,8aS)-6-(3-(3-Methyl-4-(trifluoromethoxy)phenyl)azetidine-1-carbonyl)hexahydro-2H-pyrido[4,3-b][1,4]oxazin-3(4H)-one;
(4aR,8aS)-6-(3-(3,5-Difluoro-4-(trifluoromethoxy)phenyl)azetidine-1-carbonyl)hexahydro-2H-pyrido[4,3-b][1,4]oxazin-3(4H)-one;
(4aR,8aS)-6-(3-(2-Chloro-4-(trifluoromethoxy)phenyl)azetidine-1-carbonyl)hexahydro-2H-pyrido[4,3-b][1,4]oxazin-3(4H)-one;
(4aR,8aS)-6-(3-(4-(Bicyclo[1.1.1]pentan-1-yl)phenyl)azetidine-1-carbonyl)hexahydro-2H-pyrido[4,3-b][1,4]oxazin-3(4H)-one;
(4aR,8aS)-6-(3-(5-(2-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-Fluoro-1H-indol-3-yl)pyrrolidine-1-carbonyl)hexahydro-2H-pyrido[4,3-b][1,4]oxazin-3(4H)-one;
(4aR,8aS)-6-(3-(2-Fluoro-4-(trifluoromethyl)phenyl)azetidine-1-carbonyl)hexahydro-2H-pyrido[4,3-b][1,4]oxazin-3(4H)-one;
(4aR,8aS)-6-(3-(3-Chloro-4-(trifluoromethoxy)phenyl)azetidine-1-carbonyl)hexahydro-2H-pyrido[4,3-b][1,4]oxazin-3(4H)-one;
(4aR,8aS)-6-(3-(1-Methyl-1H-indazol-4-yl)azetidine-1-carbonyl)hexahydro-2H-pyrido[4,3-b][1,4]oxazin-3(4H)-one;
(4aR,8aS)-6-(3-(4-(3-Fluoropyrrolidin-1-yl)phenyl)azetidine-1-carbonyl)hexahydro-2H-pyrido[4,3-b][1,4]oxazin-3(4H)-one;
(4aR,8aS)-6-[3-[4-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-(3-(1-Methyl-1H-indazol-6-yl)azetidine-1-carbonyl)hexahydro-2H-pyrido[4,3-b][1,4]oxazin-3(4H)-one;
(4aR,8aS)-6-[3-(S or R)-[3-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-[3-(R or S)-[3-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-[3-[4-(Oxetan-3-yl)phenyl]azetidine-1-carbonyl]-4,4a,5,7,8,8a-hexahydropyrido[4,3-b][1,4]oxazin-3-one;
(4aR,8aS)-6-(3-(3-Chloro-4-(3,3-difluoroazetidin-1-yl)phenyl)azetidine-1-carbonyl)hexahydro-2H-pyrido[4,3-b][1,4]oxazin-3(4H)-one;
(4aR,8aS)-6-[(3S or R)-3-(3-Bromophenyl)pyrrolidine-1-carbonyl]-4,4a,5,7,8,8a-hexahydropyrido[4,3-b][1,4]oxazin-3-one;
(4aR,8aS)-6-[(3R or S)-3-(3-Bromophenyl)pyrrolidine-1-carbonyl]-4,4a,5,7,8,8a-hexahydropyrido[4,3-b][1,4]oxazin-3-one;
(4aR,8aS)-6-[3-[4-(3-Azabicyclo[3.1.1]heptan-3-yl)phenyl]azetidine-1-carbonyl]-4,4a,5,7,8,8a-hexahydropyrido[4,3-b][1,4]oxazin-3-one;
(4aR,8aS)-6-(2-Methyl-3-(4-(trifluoromethoxy)phenyl)azetidine-1-carbonyl)hexahydro-2H-pyrido[4,3-b][1,4]oxazin-3(4H)-one;
(4aR,8aS)-6-(3-(3,3-Dimethyl-2,3-dihydrobenzofuran-6-yl)azetidine-1-carbonyl)hexahydro-2H-pyrido[4,3-b][1,4]oxazin-3(4H)-one;
(4aR,8aS)-6-(3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-(3-(3,5-Dichlorophenyl)pyrrolidine-1-carbonyl)hexahydro-2H-pyrido[4,3-b][1,4]oxazin-3(4H)-one;
(4aR,8aS)-6-((R or S)-3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-((S or R)-3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-(3-(4-(tert-Butyl)-3-methoxyphenyl)azetidine-1-carbonyl)hexahydro-2H-pyrido[4,3-b][1,4]oxazin-3(4H)-one;
(4aR,8aS)-6-(3-(1-Methyl-1H-indazol-5-yl)azetidine-1-carbonyl)hexahydro-2H-pyrido[4,3-b][1,4]oxazin-3(4H)-one;
(4aR,8aS)-6-(3-(4-Propylphenyl)azetidine-1-carbonyl)hexahydro-2H-pyrido[4,3-b][1,4]oxazin-3(4H)-one;
(4aR,8aS)-6-(3-(4-(Trifluoromethoxy)-3-(trifluoromethyl)phenyl)azetidine-1-carbonyl)hexahydro-2H-pyrido[4,3-b][1,4]oxazin-3(4H)-one;
(4aR,8aS)-6-[3-[4-(2,2,2-Trifluoro-1,1-dimethyl-ethyl)phenyl]azetidine-1-carbonyl]-4,4a,5,7,8,8a-hexahydropyrido[4,3-b][1,4]oxazin-3-one;
(4aR,8aS)-6-[3-(R or S)-[4-(2,2,2-Trifluoro-1-methyl-ethoxy)phenyl]azetidine-1-carbonyl]-4,4a,5,7,8,8a-hexahydropyrido[4,3-b][1,4]oxazin-3-one;
(4aR,8aS)-6-[3-[4-(3-Fluoropropyl)phenyl]azetidine-1-carbonyl]-4,4a,5,7,8,8a-hexahydropyrido[4,3-b][1,4]oxazin-3-one;
(4aR,8aS)-6-[3-(S or R)-[4-(2,2,2-Trifluoro-1-methyl-ethoxy)phenyl]azetidine-1-carbonyl]-4,4a,5,7,8,8a-hexahydropyrido[4,3-b][1,4]oxazin-3-one;
(4aR,8aS)-6-[3-(4-Cyclobutylphenyl)azetidine-1-carbonyl]-4,4a,5,7,8,8a-hexahydropyrido[4,3-b][1,4]oxazin-3-one;
(4aR,8aS)-6-[3-(3-Methoxy-4-methyl-phenyl)azetidine-1-carbonyl]-4,4a,5,7,8,8a-hexahydropyrido[4,3-b][1,4]oxazin-3-one;
(4aR,8aS)-6-[3-[5-(2,4-Dichlorophenyl)-1,3,4-oxadiazol-2-yl]azetidine-1-carbonyl]-4,4a,5,7,8,8a-hexahydropyrido[4,3-b][1,4]oxazin-3-one;
(4aR,8aS)-6-[3-[3-Fluoro-4-(trifluoromethyl)phenyl]azetidine-1-carbonyl]-4,4a,5,7,8,8a-hexahydropyrido[4,3-b][1,4]oxazin-3-one;
(4aR,8aS)-6-[3-[1-(2,4-Dichlorophenyl)pyrazol-3-yl]azetidine-1-carbonyl]-4,4a,5,7,8,8a-hexahydropyrido[4,3-b][1,4]oxazin-3-one;
(4aR,8aS)-6-[3-(4-Propoxyphenyl)azetidine-1-carbonyl]-4,4a,5,7,8,8a-hexahydropyrido[4,3-b][1,4]oxazin-3-one;
(4aR,8aS)-6-[3-(3,4-Dimethylphenyl)azetidine-1-carbonyl]-4,4a,5,7,8,8a-hexahydropyrido[4,3-b][1,4]oxazin-3-one;
(4aR,8aS)-6-[3-[4-(2,2-Dimethylpropyl)phenyl]azetidine-1-carbonyl]-4,4a,5,7,8,8a-hexahydropyrido[4,3-b][1,4]oxazin-3-one;
(4aR,8aS)-6-[3-(4-tert-Butoxyphenyl)azetidine-1-carbonyl]-4,4a,5,7,8,8a-hexahydropyrido[4,3-b][1,4]oxazin-3-one;
(4aR,8aS)-6-[4-(5-Chloroindolin-1-yl)piperidine-1-carbonyl]-4,4a,5,7,8,8a-hexahydropyrido[4,3-b][1,4]oxazin-3-one;
(4aR,8aS)-6-[4-(4-Chloroisoindolin-2-yl)piperidine-1-carbonyl]-4,4a,5,7,8,8a-hexahydropyrido[4,3-b][1,4]oxazin-3-one;
(4aR,8aS)-6-[4-(5′-Chlorospiro[cyclopropane-1,3′-indoline]-1′-yl)piperidine-1-carbonyl]-4,4a,5,7,8,8a-hexahydropyrido[4,3-b][1,4]oxazin-3-one;
(4aR,8aS)-6-[3-(4-Chloroisoindolin-2-yl)azetidine-1-carbonyl]-4,4a,5,7,8,8a-hexahydropyrido[4,3-b][1,4]oxazin-3-one;
(4aR,8aS)-6-[4-(5-Chloroisoindolin-2-yl)piperidine-1-carbonyl]-4,4a,5,7,8,8a-hexahydropyrido[4,3-b][1,4]oxazin-3-one;
(4aR,8aS)-6-(3-(5-(3-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-((R or S)-3-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-((S or R)-3-(Trifluoromethyl)pyrrolidin-1-yl)pyridin-2-yl)azetidine-1-carbonyl)hexahydro-2H-pyrido[4,3-b][1,4]oxazin-3(4H)-one;
rac-(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
rac-(4aR,8aS)-6-[3-(4-tetrahydropyran-3-ylphenyl)azetidine-1-carbonyl]-4,4a,5,7,8,8a-hexahydropyrido[4,3-b][1,4]oxazin-3-one;
(4aR,8aS)-6-[3-[2-methoxy-4-(2,2,2-trifluoroethyl)phenyl]azetidine-1-carbonyl]-4,4a,5,7,8,8a-hexahydropyrido[4,3-b][1,4]oxazin-3-one;
(4aR,8aS)-6-[3-[4-(2,2-dimethylpropoxy)phenyl]azetidine-1-carbonyl]-4,4a,5,7,8,8a-hexahydropyrido[4,3-b][1,4]oxazin-3-one;
(4aR,8aS)-6-[4-[[2-(2,2,2-trifluoroethoxy)-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[3-[4-chloro-3-(trifluoromethyl)phenoxy]azetidine-1-carbonyl]-4,4a,5,7,8,8a-hexahydropyrido[4,3-b][1,4]oxazin-3-one;
(4aR,8aS)-6-[4-[3-morpholino-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-[3-cyclopropyl-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-(4-chlorophenoxy)piperidine-1-carbonyl]-4,4a,5,7,8,8a-hexahydropyrido[4,3-b][1,4]oxazin-3-one;
(4aR,8aS)-6-[4-[[2,6-difluoro-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[4-chloro-3-(4-chlorophenyl)-2-fluoro-phenoxy]piperidine-1-carbonyl]-4,4a,5,7,8,8a-hexahydropyrido[4,3-b][1,4]oxazin-3-one;
(4aR,8aS)-6-[3-[2-chloro-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[[2-fluoro-6-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-methyl-2-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[6-fluoro-4-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-[[6-fluoro-5-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-[(3,4-dichlorophenyl)methoxy]azetidine-1-carbonyl]-4,4a,5,7,8,8a-hexahydropyrido[4,3-b][1,4]oxazin-3-one;
(4aR,8aS)-6-[3-[(2,5-dichlorophenyl)methoxy]azetidine-1-carbonyl]-4,4a,5,7,8,8a-hexahydropyrido[4,3-b][1,4]oxazin-3-one;
(4aR,8aS)-6-[3-[[3-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[5-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-methyl-4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[3-methyl-4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[4,5-bis(trifluoromethyl)-2-pyridyl]oxymethyl]azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[2-fluoro-4-(pentafluoro-lambda6-sulfanyl)phenyl]methoxy]azetidine-1-carbonyl]-4,4a,5,7,8,8a-hexahydropyrido[4,3-b][1,4]oxazin-3-one;
(4aR,8aS)-6-[3-[[4-(4-fluorophenyl)thiazol-2-yl]methoxy]azetidine-1-carbonyl]-4,4a,5,7,8,8a-hexahydropyrido[4,3-b][1,4]oxazin-3-one;
rac-(4aR,8aS)-6-[rac-(2R,3S)-3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
(4aR,8aS)-6-[3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
(4aR,8aS)-6-[3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
6-[3-[[2,4-bis(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methyl-4-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[2-methyl-4-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[2-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[2-fluoro-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-[3-chloro-4-(trifluoromethyl)phenoxy]piperidine-1-carbonyl]-4,4a,5,7,8,8a-hexahydropyrido[4,3-b][1,4]oxazin-3-one;
(4aR,8aS)-6-[3-(4-chloro-3-cyclopropyl-phenoxy)azetidine-1-carbonyl]-4,4a,5,7,8,8a-hexahydropyrido[4,3-b][1,4]oxazin-3-one;
(4aR,8aS)-6-[4-[2-chloro-3-(trifluoromethyl)phenoxy]piperidine-1-carbonyl]-4,4a,5,7,8,8a-hexahydropyrido[4,3-b][1,4]oxazin-3-one;
(4aR,8aS)-6-[3-(3-bromo-2-chloro-phenoxy)azetidine-1-carbonyl]-4,4a,5,7,8,8a-hexahydropyrido[4,3-b][1,4]oxazin-3-one;
(4aR,8aS)-6-[3-(2-chloro-3-cyclopropyl-phenoxy)azetidine-1-carbonyl]-4,4a,5,7,8,8a-hexahydropyrido[4,3-b][1,4]oxazin-3-one;
(4aR,8aS)-6-[3-[3-cyclopropyl-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-chloro-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-(3-bromo-4-chloro-phenoxy)azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(3,3-difluoroazetidin-1-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(6,6-difluoro-2-azaspiro[3.3]heptan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(5-oxa-2-azaspiro[3.5]nonan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-2-chloro-phenoxy]azetidine-1-carbonyl]-4,4a,5,7,8,8a-hexahydropyrido[4,3-b][1,4]oxazin-3-one;
(4aR,8aS)-6-[3-[2-chloro-3-(5-oxa-2-azaspiro[3.4]octan-2-yl)phenoxy]azetidine-1-carbonyl]-4,4a,5,7,8,8a-hexahydropyrido[4,3-b][1,4]oxazin-3-one;
(4aR,8aS)-6-[3-[2-chloro-3-(5-oxa-2-azaspiro[3.5]nonan-2-yl)phenoxy]azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-5-chloro-phenoxy]azetidine-1-carbonyl]-4,4a,5,7,8,8a-hexahydropyrido[4,3-b][1,4]oxazin-3-one;
(4aR,8aS)-6-[3-(3-chloro-5-pyrrolidin-1-yl-phenoxy)azetidine-1-carbonyl]-4,4a,5,7,8,8a-hexahydropyrido[4,3-b][1,4]oxazin-3-one;
(4aR,8aS)-6-[3-[[4-fluoro-2-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-(trifluoromethoxy)phenyl]methyl]azetidine-1-carbonyl]-4,4a,5,7,8,8a-hexahydropyrido[4,3-b][1,4]oxazin-3-one;
rac-(4aR,8aS)-6-[3-[2-fluoro-5-(trifluoromethyl)phenoxy]pyrrolidine-1-carbonyl]-4,4a,5,7,8,8a-hexahydropyrido[4,3-b][1,4]oxazin-3-one;
rac-(4aR,8aS)-6-[3-[2-chloro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[(3S)-3-[2-fluoro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[(3R)-3-[2-fluoro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[3-[[3-fluoro-4-(trifluoromethoxy)phenyl]methoxy]azetidine-1-carbonyl]-4,4a,5,7,8,8a-hexahydropyrido[4,3-b][1,4]oxazin-3-one;
(4aR,8aS)-6-[3-[(2,3-dimethylphenyl)methoxy]azetidine-1-carbonyl]-4,4a,5,7,8,8a-hexahydropyrido[4,3-b][1,4]oxazin-3-one;
(4aR,8aS)-6-[3-[(2,4-dimethylphenyl)methoxy]azetidine-1-carbonyl]-4,4a,5,7,8,8a-hexahydropyrido[4,3-b][1,4]oxazin-3-one;
(4aR,8aS)-6-[3-[[2-methyl-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[(4-tert-butylthiazol-2-yl)methyl]piperidine-1-carbonyl]-4,4a,5,7,8,8a-hexahydropyrido[4,3-b][1,4]oxazin-3-one;
(4aR,8aS)-6-[4-[(4-tert-butyloxazol-2-yl)methyl]piperidine-1-carbonyl]-4,4a,5,7,8,8a-hexahydropyrido[4,3-b][1,4]oxazin-3-one;
rac-(4aR,8aS)-6-[4-[(4-tert-butylthiazol-2-yl)methyl]piperidine-1-carbonyl]-4,4a,5,7,8,8a-hexahydropyrido[4,3-b][1,4]oxazin-3-one;
rac-(4aR,8aS)-6-[4-[(4-tert-butyloxazol-2-yl)methyl]piperidine-1-carbonyl]-4,4a,5,7,8,8a-hexahydropyrido[4,3-b][1,4]oxazin-3-one;
(4aS,8aR)-6-[4-[(4-tert-butylthiazol-2-yl)methyl]piperidine-1-carbonyl]-4,4a,5,7,8,8a-hexahydropyrido[4,3-b][1,4]oxazin-3-one;
(4aS,8aR)-6-[4-[(4-tert-butyloxazol-2-yl)methyl]piperidine-1-carbonyl]-4,4a,5,7,8,8a-hexahydropyrido[4,3-b][1,4]oxazin-3-one;
rac-(4aR,8aS)-6-[4-[(2-chloro-4-fluoro-phenoxy)methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[(4-chlorophenoxy)methyl]piperidine-1-carbonyl]-4,4a,5,7,8,8a-hexahydropyrido[4,3-b][1,4]oxazin-3-one;
rac-(4aR,8aS)-6-[4-[(4-chlorophenyl)methyl]piperidine-1-carbonyl]-4,4a,5,7,8,8a-hexahydropyrido[4,3-b][1,4]oxazin-3-one;
rac-(4aR,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[4-(trifluoromethyl)phenoxy]piperidine-1-carbonyl]-4,4a,5,7,8,8a-hexahydropyrido[4,3-b][1,4]oxazin-3-one;
rac-(4aR,8aS)-6-[4-[4-(trifluoromethyl)phenoxy]piperidine-1-carbonyl]-4,4a,5,7,8,8a-hexahydropyrido[4,3-b][1,4]oxazin-3-one;
(4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-(phenoxymethyl)piperidine-1-carbonyl]-4,4a,5,7,8,8a-hexahydropyrido[4,3-b][1,4]oxazin-3-one;
rac-(4aR,8aS)-6-[4-(5,6-dihydro-4H-cyclopenta[d]thiazol-2-ylmethyl)piperidine-1-carbonyl]-4,4a,5,7,8,8a-hexahydropyrido[4,3-b][1,4]oxazin-3-one;
rac-(4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[(3-phenyl-1,2,4-oxadiazol-5-yl)methyl]piperidine-1-carbonyl]-4,4a,5,7,8,8a-hexahydropyrido[4,3-b][1,4]oxazin-3-one;
(4aR,8aR)-6-[4-[[4-(trifluoromethyl)phenyl]methyl]piperidine-1-carbonyl]-4,4a,5,7,8,8a-hexahydropyrido[4,3-b][1,4]oxazin-3-one;
(4aS,8aR)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[[4-(trifluoromethyl)phenyl]methyl]piperazine-1-carbonyl]-4,4a,5,7,8,8a-hexahydropyrido[4,3-b][1,4]oxazin-3-one;
rac-(4aR,8aS)-6-[4-[(4-chlorophenyl)methyl]piperazine-1-carbonyl]-4,4a,5,7,8,8a-hexahydropyrido[4,3-b][1,4]oxazin-3-one;
(4aS,8aR)-6-[4-[(2-chloro-4-fluoro-phenoxy)methyl]piperidine-1-carbonyl]-4,4a,5,7,8,8a-hexahydropyrido[4,3-b][1,4]oxazin-3-one;
(4aR,8aS)-6-[4-[(2-chloro-4-fluoro-phenoxy)methyl]piperidine-1-carbonyl]-4,4a,5,7,8,8a-hexahydropyrido[4,3-b][1,4]oxazin-3-one;
rac-(4aR,8aS)-6-[4-[[5-(trifluoromethyl)-2-pyridyl]methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)pyrazol-1-yl]methyl]piperidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[(2-chloro-4-fluoro-phenyl)methoxy]azetidine-1-carbonyl]-4,4a,5,7,8,8a-hexahydropyrido[4,3-b][1,4]oxazin-3-one;
(4aR,8aS)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aS,8aR)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[[4-(trifluoromethoxy)phenyl]methyl]piperidine-1-carbonyl]-4,4a,5,7,8,8a-hexahydropyrido[4,3-b][1,4]oxazin-3-one;
(4aR,8aS)-6-[4-[(2,4-difluorophenoxy)methyl]piperidine-1-carbonyl]-4,4a,5,7,8,8a-hexahydropyrido[4,3-b][1,4]oxazin-3-one;
(4aR,8aS)-6-[4-[(4-chloro-3-fluoro-phenyl)methyl]piperidine-1-carbonyl]-4,4a,5,7,8,8a-hexahydropyrido[4,3-b][1,4]oxazin-3-one;
(4aR,8aS)-6-[4-[(4-chlorophenyl)methyl]piperidine-1-carbonyl]-4,4a,5,7,8,8a-hexahydropyrido[4,3-b][1,4]oxazin-3-one;
(4aR,8aS)-6-[3-[[4-(trifluoromethyl)phenyl]methyl]azetidine-1-carbonyl]-4,4a,5,7,8,8a-hexahydropyrido[4,3-b][1,4]oxazin-3-one;
(4aR,8aS)-6-[4-[(4,4-difluoro-1-piperidyl)methyl]piperidine-1-carbonyl]-4,4a,5,7,8,8a-hexahydropyrido[4,3-b][1,4]oxazin-3-one;
(4aR,8aS)-6-[4-[(5-tert-butyloxazol-2-yl)methyl]piperidine-1-carbonyl]-4,4a,5,7,8,8a-hexahydropyrido[4,3-b][1,4]oxazin-3-one;
(4aR,8aS)-6-[4-[(2-fluoro-4-methoxy-phenoxy)methyl]piperidine-1-carbonyl]-4,4a,5,7,8,8a-hexahydropyrido[4,3-b][1,4]oxazin-3-one;
(4aR,8aS)-6-[4-[2-chloro-4-(trifluoromethyl)phenoxy]piperidine-1-carbonyl]-4,4a,5,7,8,8a-hexahydropyrido[4,3-b][1,4]oxazin-3-one;
(4aS,8aR)-6-[4-[[6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[4-[[6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[4-[3-(trifluoromethyl)phenoxy]piperidine-1-carbonyl]-4,4a,5,7,8,8a-hexahydropyrido[4,3-b][1,4]oxazin-3-one;
(4aS,8aR)-6-[4-[[2-chloro-4-(trifluoromethoxy)phenoxy]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethoxy)phenoxy]methyl]piperidine-1-carbonyl]-4,4a,5,7,8,8a-hexahydropyrido[4,3-b][1,4]oxazin-3-one;
(4aR,8aS)-6-[3-[[2-fluoro-4-(trifluoromethyl)phenoxy]methyl]azetidine-1-carbonyl]-4,4a,5,7,8,8a-hexahydropyrido[4,3-b][1,4]oxazin-3-one;
(4aS,8aR)-6-[4-[(2,4-difluorophenoxy)methyl]piperidine-1-carbonyl]-4,4a,5,7,8,8a-hexahydropyrido[4,3-b][1,4]oxazin-3-one;
(4aR,8aS)-6-[4-[(4-chloro-2-fluoro-phenoxy)methyl]piperidine-1-carbonyl]-4,4a,5,7,8,8a-hexahydropyrido[4,3-b][1,4]oxazin-3-one;
(4aR,8aS)-6-[4-[[4-fluoro-2-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[4-[[2-fluoro-4-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[4-[[2-pyrrolidin-1-yl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[3-[(2-chloro-4-fluoro-phenoxy)methyl]azetidine-1-carbonyl]-4,4a,5,7,8,8a-hexahydropyrido[4,3-b][1,4]oxazin-3-one;
(4aR,8aS)-6-[4-[[2-cyclopentyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)imidazol-1-yl]methyl]piperidine-1-carbonyl]-4,4a,5,7,8,8a-hexahydropyrido[4,3-b][1,4]oxazin-3-one;
(4aR,8aS)-6-[4-[(4-fluoro-2-methyl-phenoxy)methyl]piperidine-1-carbonyl]-4,4a,5,7,8,8a-hexahydropyrido[4,3-b][1,4]oxazin-3-one;
(4aR,8aS)-6-[3-[[2-chloro-4-(trifluoromethyl)phenoxy]methyl]azetidine-1-carbonyl]-4,4a,5,7,8,8a-hexahydropyrido[4,3-b][1,4]oxazin-3-one;
(4aR,8aS)-6-[4-[(4-tert-butylpyrazol-1-yl)methyl]piperidine-1-carbonyl]-4,4a,5,7,8,8a-hexahydropyrido[4,3-b][1,4]oxazin-3-one;
(2R,4aR,8aS)-2-methyl-6-[4-[[4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-(1,3-benzoxazol-2-ylmethyl)piperidine-1-carbonyl]-4,4a,5,7,8,8a-hexahydropyrido[4,3-b][1,4]oxazin-3-one;
(4aR,8aS)-6-[4-[4-chloro-3-(4-chlorophenyl)phenoxy]piperidine-1-carbonyl]-4,4a,5,7,8,8a-hexahydropyrido[4,3-b][1,4]oxazin-3-one;
(4aR,8aS)-6-[4-[(2-chloro-4-fluoro-phenoxy)methyl]-3-methyl-piperidine-1-carbonyl]-4,4a,5,7,8,8a-hexahydropyrido[4,3-b][1,4]oxazin-3-one;
(4aR,8aS)-6-[3-[[2-chloro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-fluoro-4-(trifluoromethoxy)phenyl]methoxy]azetidine-1-carbonyl]-4,4a,5,7,8,8a-hexahydropyrido[4,3-b][1,4]oxazin-3-one;
(4aR,8aS)-6-[4-[[2-(1H-pyrazol-4-yl)-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[3-[(2,4-dichlorophenyl)methoxy]azetidine-1-carbonyl]-4,4a,5,7,8,8a-hexahydropyrido[4,3-b][1,4]oxazin-3-one;
(4aR,8aS)-6-[3-[[3-methoxy-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
rac-(4aR,8aS)-6-[3-[(3-chlorophenoxy)methyl]pyrrolidine-1-carbonyl]-4,4a,5,7,8,8a-hexahydropyrido[4,3-b][1,4]oxazin-3-one;
rac-(4aR,8aS)-6-[3-[(2-chlorophenoxy)methyl]pyrrolidine-1-carbonyl]-4,4a,5,7,8,8a-hexahydropyrido[4,3-b][1,4]oxazin-3-one;
(4aR,8aS)-6-[4-[[2-fluoro-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[(2-chlorophenyl)methoxy]pyrrolidine-1-carbonyl]-4,4a,5,7,8,8a-hexahydropyrido[4,3-b][1,4]oxazin-3-one;
rac-(4aR,8aS)-6-[3-[(3-chlorophenyl)methoxy]pyrrolidine-1-carbonyl]-4,4a,5,7,8,8a-hexahydropyrido[4,3-b][1,4]oxazin-3-one;
(4aR,8aS)-6-[4-[[2-cyclopropyl-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[(4-chlorophenoxy)methyl]pyrrolidine-1-carbonyl]-4,4a,5,7,8,8a-hexahydropyrido[4,3-b][1,4]oxazin-3-one;
rac-(4aR,8aS)-6-[3-[(4-chlorophenyl)methoxy]pyrrolidine-1-carbonyl]-4,4a,5,7,8,8a-hexahydropyrido[4,3-b][1,4]oxazin-3-one;
(4aR,8aS)-6-[4-[[2-methyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[[4-(trifluoromethyl)phenyl]methyl]pyrrolidine-1-carbonyl]-4,4a,5,7,8,8a-hexahydropyrido[4,3-b][1,4]oxazin-3-one;
(4aR,8aS)-6-[3-[[3-fluoro-5-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-chloro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2,4-difluoro-5-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-fluoro-5-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methoxy-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-chloro-2-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[4-(trifluoromethyl)phenoxy]azetidine-1-carbonyl]-4,4a,5,7,8,8a-hexahydropyrido[4,3-b][1,4]oxazin-3-one;
(4aR,8aS)-6-[4-[4-chloro-3-(trifluoromethyl)phenoxy]piperidine-1-carbonyl]-4,4a,5,7,8,8a-hexahydropyrido[4,3-b][1,4]oxazin-3-one;
(4aR,8aS)-6-[4-(4-chloro-3-cyclopropyl-phenoxy)piperidine-1-carbonyl]-4,4a,5,7,8,8a-hexahydropyrido[4,3-b][1,4]oxazin-3-one;
(4aR,8aS)-6-[4-(4-chloro-3-morpholino-phenoxy)piperidine-1-carbonyl]-4,4a,5,7,8,8a-hexahydropyrido[4,3-b][1,4]oxazin-3-one;
(4aR,8aS)-6-[4-[2-methyl-4-(trifluoromethyl)phenoxy]piperidine-1-carbonyl]-4,4a,5,7,8,8a-hexahydropyrido[4,3-b][1,4]oxazin-3-one; and
(4aR,8aS)-6-[4-(oxazolo[5,4-c]pyridin-2-ylmethyl)piperidine-1-carbonyl]-4,4a,5,7,8,8a-hexahydropyrido[4,3-b][1,4]oxazin-3-one.
E2. The compound of formula (I) according to A1 or E1, or a pharmaceutically acceptable salt thereof, wherein X is N.
E3. The compound of formula (I) according to any one of A1 and E1 to E2, or a pharmaceutically acceptable salt thereof, wherein A is C₆-C₁₄-aryl.
E4. The compound of formula (I) according to any one of A1 and E1 to E2, or a pharmaceutically acceptable salt thereof, wherein A is phenyl.
E5. The compound of formula (I) according to any one of A1 and E1 to E4, or a pharmaceutically acceptable salt thereof, wherein L¹ is selected from a covalent bond, —CH₂—, —OCHR^{L}—, —CH₂O—, and —NHC(O)—.
E6. The compound of formula (I) according to any one of A1 and E1 to E4, or a pharmaceutically acceptable salt thereof, wherein L¹ is selected from a covalent bond, —CH₂O—, —OCH₂—, and —CH₂—.
E7. The compound of formula (I) according to any one of A1 and E1 to E4, or a pharmaceutically acceptable salt thereof, wherein L¹ is selected from a covalent bond, —CH₂O—, and —CH₂—.
E8. The compound of formula (I) according to any one of A1 and E1 to E7, or a pharmaceutically acceptable salt thereof, wherein B is
   (i) C₆-C₁₄-aryl or 3-14 membered heterocyclyl and L² is a covalent bond; or
   (ii) C₃-C₁₀-cycloalkyl and L² is selected from a covalent bond, —O—, and —CH₂O—.
E9. The compound of formula (I) according to any one of A1 and E1 to E7, or a pharmaceutically acceptable salt thereof, wherein B is
   (i) 3-14 membered heterocyclyl and L² is a covalent bond; or
   (ii) C₃-C₁₀-cycloalkyl and L² is selected from a covalent bond, —O—, and —CH₂O—.
E10. The compound of formula (I) according to any one of A1 and E1 to E7, or a pharmaceutically acceptable salt thereof, wherein B is
   (i) tetrahydropyranyl and L² is a covalent bond; or
   (ii) cyclopropyl or cyclobutyl and L² is selected from a covalent bond, —O—, and —CH₂O—.
E11. The compound of formula (I) according to any one of A1 and E1 to E10, or a pharmaceutically acceptable salt thereof, wherein R¹ is hydrogen or methyl.
E12. The compound of formula (I) according to any one of A1 and E1 to E10, or a pharmaceutically acceptable salt thereof, wherein R¹ is hydrogen.
E13. The compound of formula (I) according to any one of A1 and E1 to E12, or a pharmaceutically acceptable salt thereof, wherein R² is selected from a group

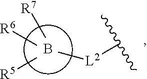

C₁-C₆-alkyl, C₁-C₆-alkoxy, halo-C₁-C₆-alkyl, halo-C₁-C₆-alkoxy, halogen, and SF₅.
E14. The compound of formula (I) according to any one of A1 and E1 to E12, or a pharmaceutically acceptable salt thereof, wherein R² is selected from a group

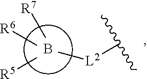

halo-C₁-C₆-alkyl, halo-C₁-C₆-alkoxy, halogen, and SF₅.
E15. The compound of formula (I) according to any one of A1 and E1 to E12, or a pharmaceutically acceptable salt thereof, wherein R² is selected from a group

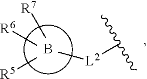

halo-C₁-C₆-alkyl, halo-C₁-C₆-alkoxy, and SF₅.
E16. The compound of formula (I) according to any one of A1 and E1 to E12, or a pharmaceutically acceptable salt thereof, wherein R² is selected from a group

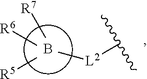

CF3, OCF₃, 2,2,2-trifluoro-1,1-dimethyl-ethoxy, fluoro, and SF₅.
E17. The compound of formula (I) according to any one of A1 and E1 to E12, or a pharmaceutically acceptable salt thereof, wherein R² is selected from a group

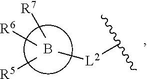

CF₃, OCF₃, 2,2,2-trifluoro-1,1-dimethyl-ethoxy, and SF₅.
E18. The compound of formula (I) according to anyone of A1 and E1 to E17, or a pharmaceutically acceptable salt thereof, wherein R³ is selected from hydrogen, halogen, C₁-C₆-alkyl, and halo-C₁-C₆-alkyl.
E19. The compound of formula (I) according to any one of A1 and E1 to E17, or a pharmaceutically acceptable salt thereof, wherein R³ is selected from hydrogen, halogen, and halo-C₁-C₆-alkyl.
E20. The compound of formula (I) according to any one of A1 and E1 to E17, or a pharmaceutically acceptable salt thereof, wherein R³ is selected from hydrogen, chloro, and CF₃.
E21. The compound of formula (I) according to any one of A1 and E1 to E20, or a pharmaceutically acceptable salt thereof, wherein R⁴ is hydrogen.
E22. The compound of formula (I) according to any one of A1 and E1 to E21, or a pharmaceutically acceptable salt thereof, wherein R⁵ is selected from hydrogen and halogen.
E23. The compound of formula (I) according to any one of A1 and E1 to E22, or a pharmaceutically acceptable salt thereof, wherein R⁵ is selected from hydrogen and fluoro.
E24. The compound of formula (I) according to any one of A1 and E1 to E23, or a pharmaceutically acceptable salt thereof, wherein R⁶ is selected from hydrogen and halogen.
E25. The compound of formula (I) according to any one of A1 and E1 to E23, or a pharmaceutically acceptable salt thereof, wherein R⁶ is selected from hydrogen and fluoro.
E26. The compound of formula (I) according to any one of A1 and E1 to E25, or a pharmaceutically acceptable salt thereof, wherein R⁷ is hydrogen.
E27. The compound of formula (I) according to A1, or a pharmaceutically acceptable salt thereof, wherein the compound of formula (I) is a compound of formula (IIa)

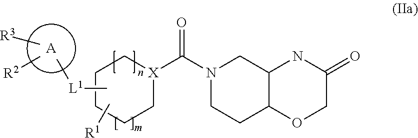

wherein:
X is CH or N;
m and n are both 1; or
m and n are both 0;
A is selected from C₆-C₁₄-aryl and 5-14 membered heteroaryl;
L¹ is selected from a covalent bond, —CH₂—, —OCHR^{L}—, —CH₂O—, and —NHC(O)—;
R^{L} is selected from hydrogen and C₁-C₆-alkyl;
R¹ is hydrogen or C₁-C₆-alkyl;
R² is selected from a group

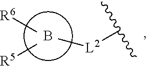

C₁-C₆-alkyl, C₁-C₆-alkoxy, halo-C₁-C₆-alkyl, halo-C₁-C₆-alkoxy, halogen, and SF₅;
R³ is selected from hydrogen, halogen, C₁-C₆-alkyl, and halo-C₁-C₆-alkyl;
B is
   (i) C₆-C₁₄-aryl or 3-14 membered heterocyclyl and L² is a covalent bond; or
   (ii) C₃-C₁₀-cycloalkyl and L² is selected from a covalent bond, —O—, and —CH₂O—;
R⁵ is selected from hydrogen, halogen, and halo-C₁-C₆-alkyl;
R⁶ is selected from hydrogen and halogen.
E28. The compound of formula (I) according to A1, or a pharmaceutically acceptable salt thereof, wherein the compound of formula (I) is a compound of formula (II)

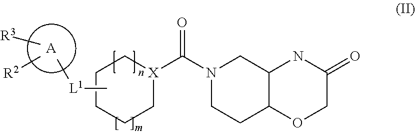

wherein:
X is CH or N;
m and n are both 1; or
m and n are both 0;
A is selected from C₆-C₁₄-aryl and 5-14 membered heteroaryl;
L¹ is selected from a covalent bond, —CH₂—, —OCHR^{L}—, —CH₂O—, and —NHC(O)—;
R^{L} is selected from hydrogen and C₁-C₆-alkyl;
R² is selected from a group

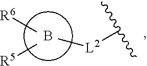

C₁-C₆-alkyl, C₁-C₆-alkoxy, halo-C₁-C₆-alkyl, halo-C₁-C₆-alkoxy, halogen, and SF₅;
R³ is selected from hydrogen, halogen, C₁-C₆-alkyl, and halo-C₁-C₆-alkyl; B is
   (i) C₆-C₁₄-aryl or 3-14 membered heterocyclyl and L² is a covalent bond; or
   (ii) C₃-C₁₀-cycloalkyl and L² is selected from a covalent bond, —O—, and —CH₂O—;
R⁵ is selected from hydrogen, halogen, and halo-C₁-C₆-alkyl;
R⁶ is selected from hydrogen and halogen.
E29. The compound of formula (I) according to A1, or a pharmaceutically acceptable salt thereof, wherein the compound of formula (I) is a compound of formula (IIIa)

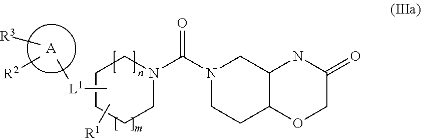

wherein:
m and n are both 1; or
m and n are both 0;
A is C₆-C₁₄-aryl;
L¹ is selected from a covalent bond, —CH₂O—, and —CH₂—;
R¹ is hydrogen or C₁-C₆-alkyl;
R² is selected from a group

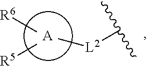

halo-C₁-C₆-alkyl, halo-C₁-C₆-alkoxy, halogen, and SF₅;
R³ is selected from hydrogen, halogen, and halo-C₁-C₆-alkyl;
B is
   (i) 3-14 membered heterocyclyl and L² is a covalent bond; or
   (ii) C₃-C₁₀-cycloalkyl and L² is selected from a covalent bond, —O—, and —CH₂O—; and
R⁵ and R⁶ are independently selected from hydrogen and halogen.
E30. The compound of formula (I) according to A1, or a pharmaceutically acceptable salt thereof, wherein the compound of formula (I) is a compound of formula (III)

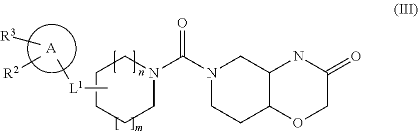

wherein:
m and n are both 1; or
m and n are both 0;
A is C₆-C₁₄-aryl;
L¹ is selected from a covalent bond, —CH₂O—, and —CH₂—;
R² is selected from a group

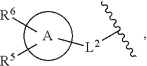

halo-C₁-C₆-alkyl, halo-C₁-C₆-alkoxy, and SF₅;
R³ is selected from hydrogen, halogen, and halo-C₁-C₆-alkyl;
B is
   (i) 3-14 membered heterocyclyl and L² is a covalent bond; or
   (ii) C₃-C₁₀-cycloalkyl and L² is selected from a covalent bond, —O—, and —CH₂O—; and
R⁵ and R⁶ are independently selected from hydrogen and halogen.
E31. The compound of formula (I) according to A1, or a pharmaceutically acceptable salt thereof, wherein the compound of formula (I) is a compound of formula (IVa)

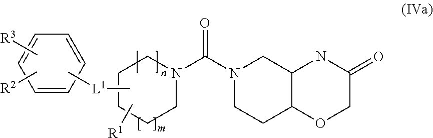

wherein:
m and n are both 1; or
m and n are both 0;
L¹ is selected from a covalent bond, —CH₂O—, —OCH₂—, and —CH₂—;
R¹ is hydrogen or methyl;
R² is selected from a group

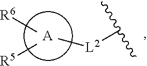

CF₃, OCF₃, 2,2,2-trifluoro-1,1-dimethyl-ethoxy, fluoro, and SF₅;
R³ is selected from hydrogen, chloro, and CF₃;
B is
   (i) tetrahydropyranyl and L² is a covalent bond; or
   (ii) cyclopropyl or cyclobutyl and L² is selected from a covalent bond, —O—, and —CH₂O—; and
R⁵ and R⁶ are independently selected from hydrogen and fluoro.
E32. The compound of formula (I) according to A1, or a pharmaceutically acceptable salt thereof, wherein the compound of formula (I) is a compound of formula (IV)

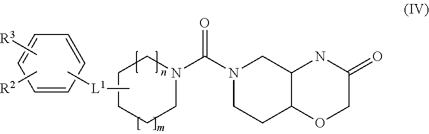

wherein:
m and n are both 1; or
m and n are both 0;
L¹ is selected from a covalent bond, —CH₂O—, and —CH₂—;
R² is selected from a group

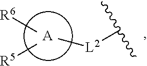

CF₃, OCF₃, 2,2,2-trifluoro-1,1-dimethyl-ethoxy, and SF₅;
R³ is selected from hydrogen, chloro, and CF₃;
B is
   (i) tetrahydropyranyl and L² is a covalent bond; or
   (ii) cyclopropyl or cyclobutyl and L² is selected from a covalent bond, —O—, and —CH₂O—; and
R⁵ and R⁶ are independently selected from hydrogen and fluoro.
E33. The compound of formula (I) according to A1, or a pharmaceutically acceptable salt thereof, wherein:
   X is CH or N;
   m and n are both 1; or
   m and n are both 0; and
   R¹ is hydrogen or C₁-C₆-alkyl.
E34. The compound of formula (I) according to A1, or a pharmaceutically acceptable salt thereof, wherein:
   X is N;
   m and n are both 1; or
   m and n are both 0; and
   R¹ is hydrogen or C₁-C₆-alkyl.
E35. The compound of formula (I) according to A1, or a pharmaceutically acceptable salt thereof, wherein:
   X is N;
   m and n are both 1; or
   m and n are both 0; and
   R¹ is hydrogen or methyl.
E36. The compound of formula (I) according to A1 and E33 to E35, or a pharmaceutically acceptable salt thereof, wherein:
   A is selected from C₆-C₁₄-aryl and 5-14 membered heteroaryl;
   L¹ is selected from a covalent bond, —CH₂—, —OCHR^{L}—, —CH₂O—, and —NHC(O)—;
   R^{L} is selected from hydrogen and C₁-C₆-alkyl;
   R² is selected from a group

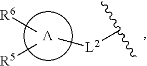

C₁-C₆-alkyl, C₁-C₆-alkoxy, halo-C₁-C₆-alkyl, halo-C₁-C₆-alkoxy, halogen, and SF₅;
R³ is selected from hydrogen, halogen, C₁-C₆-alkyl, and halo-C₁-C₆-alkyl;
R⁴ is hydrogen;
B is
   (i) C₆-C₁₄-aryl or 3-14 membered heterocyclyl and L² is a covalent bond; or
   (ii) C₃-C₁₀-cycloalkyl and L² is selected from a covalent bond, —O—, and —CH₂O—;
R⁵ is selected from hydrogen, halogen, and halo-C₁-C₆-alkyl;
R⁶ is selected from hydrogen and halogen.
E37. The compound of formula (I) according to A1 and E33 to E35, or a pharmaceutically acceptable salt thereof, wherein:
   A is C₆-C₁₄-aryl;
   L¹ is selected from a covalent bond, —CH₂O—, and —CH₂—;
   R² is selected from a group

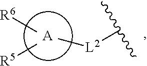

halo-C₁-C₆-alkyl, halo-C₁-C₆-alkoxy, halogen, and SF₅;
R⁴ is hydrogen;
R³ is selected from hydrogen, halogen, and halo-C₁-C₆-alkyl;
B is
   (i) 3-14 membered heterocyclyl and L² is a covalent bond; or
   (ii) C₃-C₁₀-cycloalkyl and L² is selected from a covalent bond, —O—, and —CH₂O—; and
R⁵ and R⁶ are independently selected from hydrogen and halogen.
E38. The compound of formula (I) according to A1 and E33 to E35, or a pharmaceutically acceptable salt thereof, wherein:
   A is phenyl;
   L¹ is selected from a covalent bond, —CH₂O—, —OCH₂—, and —CH₂—;
   R² is selected from a group

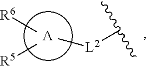

CF₃, OCF₃, 2,2,2-trifluoro-1,1-dimethyl-ethoxy, fluoro, and SF₅;
R³ is selected from hydrogen, chloro, and CF₃;
R⁴ is hydrogen;
B is
   (i) tetrahydropyranyl and L² is a covalent bond; or
   (ii) cyclopropyl or cyclobutyl and L² is selected from a covalent bond, —O—, and —CH₂O—; and
R⁵ and R⁶ are independently selected from hydrogen and fluoro.
E39. The compound of formula (I) according to any one of A1 and E1 to E25, or a pharmaceutically acceptable salt thereof, selected from:
(+)- or (−)-(4aR,8aS)-6-[3-(4-Tetrahydrofuran-3-ylphenyl)azetidine-1-carbonyl]-4,4a,5,7,8,8a-hexahydropyrido[4,3-b][1,4]oxazin-3-one;
(−)- or (+)-(4aR,8aS)-6-[3-(4-Tetrahydrofuran-3-ylphenyl)azetidine-1-carbonyl]-4,4a,5,7,8,8a-hexahydropyrido[4,3-b][1,4]oxazin-3-one;
(4aR,8aS)-6-[3-[4-(3,3-Difluorocyclobutoxy)phenyl]azetidine-1-carbonyl]-4,4a,5,7,8,8a-hexahydropyrido[4,3-b][1,4]oxazin-3-one;
(4aR,8aS)-6-[3-(4-Isopropoxyphenyl)azetidine-1-carbonyl]-4,4a,5,7,8,8a-hexahydropyrido[4,3-b][1,4]oxazin-3-one;
(4aR,8aS)-6-[3-(4-tetrahydropyran-4-ylphenyl)azetidine-1-carbonyl]-4,4a,5,7,8,8a-hexahydropyrido[4,3-b][1,4]oxazin-3-one;
(4aR,8aS)-6-[3-[4-(3,3-difluorocyclobutyl)phenyl]azetidine-1-carbonyl]-4,4a,5,7,8,8a-hexahydropyrido[4,3-b][1,4]oxazin-3-one;
(4aR,8aS)-6-[3-[5-(2,4-dichlorophenyl)-1,2,4-thiadiazol-3-yl]azetidine-1-carbonyl]-4,4a,5,7,8,8a-hexahydropyrido[4,3-b][1,4]oxazin-3-one;
(4aR,8aS)-6-[3-[1-(2,4-dichlorophenyl)imidazol-4-yl]azetidine-1-carbonyl]-4,4a,5,7,8,8a-hexahydropyrido[4,3-b][1,4]oxazin-3-one;
(4aR,8aS)-6-[3-[3-[4-chloro-2-(trifluoromethyl)phenyl]-1,2,4-oxadiazol-5-yl]azetidine-1-carbonyl]-4,4a,5,7,8,8a-hexahydropyrido[4,3-b][1,4]oxazin-3-one;
(4aR,8aS)-6-(3-(2,2-dimethylchroman-6-yl)azetidine-1-carbonyl)hexahydro-2H-pyrido[4,3-b][1,4]oxazin-3(4H)-one;
(4aR,8aS)-6-(3-(4-isobutoxyphenyl)azetidine-1-carbonyl)hexahydro-2H-pyrido[4,3-b][1,4]oxazin-3(4H)-one;
(4aR,8aS)-6-[3-[4-(2,2,2-trifluoro-1,1-dimethyl-ethoxy)phenyl]azetidine-1-carbonyl]-4,4a,5,7,8,8a-hexahydropyrido[4,3-b][1,4]oxazin-3-one;
(4aR,8aS)-6-(3-(4-(cyclopentyloxy)phenyl)azetidine-1-carbonyl)hexahydro-2H-pyrido[4,3-b][1,4]oxazin-3(4H)-one;
(4aR,8aS)-6-(3-(4,4-Dimethylchroman-6-yl)azetidine-1-carbonyl)hexahydro-2H-pyrido[4,3-b][1,4]oxazin-3(4H)-one;
(4aR,8aS)-6-(3-(4-(Cyclopropylmethoxy)phenyl)azetidine-1-carbonyl)hexahydro-2H-pyrido[4,3-b][1,4]oxazin-3(4H)-one;
(4aR,8aS)-6-[3-[3-(2,4-dichlorophenyl)-1,2,4-oxadiazol-5-yl]azetidine-1-carbonyl]-4,4a,5,7,8,8a-hexahydropyrido[4,3-b][1,4]oxazin-3-one;
(4aR,8aS)-6-(3-(1-methyl-1H-indazol-5-yl)azetidine-1-carbonyl)hexahydro-2H-pyrido[4,3-b][1,4]oxazin-3(4H)-one;
(4aR,8aS)-6-(3-(5-(2,4-dichlorophenyl)-1,2,4-oxadiazol-3-yl)azetidine-1-carbonyl)hexahydro-2H-pyrido[4,3-b][1,4]oxazin-3(4H)-one;
(4aR,8aS)-6-(3-(3-(trifluoromethoxy)phenyl)azetidine-1-carbonyl)hexahydro-2H-pyrido[4,3-b][1,4]oxazin-3(4H)-one;
(4aR,8aS)-6-(4-(1-(4-fluorophenyl)-1H-pyrazol-3-yl)piperidine-1-carbonyl)hexahydro-2H-pyrido[4,3-b][1,4]oxazin-3(4H)-one;
(4aR,8aS)-6-(3-((2,6-Dichlorobenzyl)oxy)azetidine-1-carbonyl)hexahydro-2H-pyrido[4,3-b][1,4]oxazin-3(4H)-one;
(4aR,8aS)-6-(3-((3,5-dichlorobenzyl)oxy)azetidine-1-carbonyl)hexahydro-2H-pyrido[4,3-b][1,4]oxazin-3(4H)-one;
(4aR,8aS)-6-(4-((5-cyclopropyl-4-methylpyridin-3-yl)methyl)piperidine-1-carbonyl)hexahydro-2H-pyrido[4,3-b][1,4]oxazin-3(4H)-one;
(4aR,8aS)-6-(4-((4-(trifluoromethyl)benzyl)oxy)piperidine-1-carbonyl)hexahydro-2H-pyrido[4,3-b][1,4]oxazin-3(4H)-one;
(4aR,8aS)-6-(4-((2-chloro-4-fluorobenzyl)oxy)piperidine-1-carbonyl)hexahydro-2H-pyrido[4,3-b][1,4]oxazin-3(4H)-one;
(4aR,8aS)-6-(4-((2-Chloro-4-(trifluoromethyl)benzyl)oxy)piperidine-1-carbonyl)hexahydro-2H-pyrido[4,3-b][1,4]oxazin-3(4H)-one;
rac-(4aR,8aS)-6-[3-[[2-Fluoro-6-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-Fluoro-6-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[2-Fluoro-6-(trifluoromethyl)phenyl]methoxy]-2-methylazetidine-1-carbonyl]-4,4a,5,7,8,8a-hexahydropyrido[4,3-b][1,4]oxazin-3-one CHECK!;
(4aR,8aS)-6-(3-((4-fluoro-3-methoxybenzyl)oxy)azetidine-1-carbonyl)hexahydro-2H-pyrido[4,3-b][1,4]oxazin-3(4H)-one;
(4aR,8aS)-6-(3-((4-(pentafluoro-16-sulfaneyl)benzyl)oxy)azetidine-1-carbonyl)hexahydro-2H-pyrido[4,3-b][1,4]oxazin-3(4H)-one;
(4aR,8aS)-6-(3-((2-(trifluoromethoxy)benzyl)oxy)azetidine-1-carbonyl)hexahydro-2H-pyrido[4,3-b][1,4]oxazin-3(4H)-one;
(4aR,8aS)-6-(3-((4-chloro-2-(trifluoromethoxy)benzyl)oxy)azetidine-1-carbonyl)hexahydro-2H-pyrido[4,3-b][1,4]oxazin-3(4H)-one;
(4aR,8aS)-6-(3-((2-methyl-5-(trifluoromethyl)benzyl)oxy)azetidine-1-carbonyl)hexahydro-2H-pyrido[4,3-b][1,4]oxazin-3(4H)-one;
(4aR,8aS)-6-[4-[[3-phenyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2,4-bis(trifluoromethyl)phenyl]methyl]piperidine-1-carbonyl]-4,4a,5,7,8,8a-hexahydropyrido[4,3-b][1,4]oxazin-3-one;
(4aR,8aS)-6-(4-((5-methyl-6-(trifluoromethyl)pyridin-3-yl)methyl)piperidine-1-carbonyl)hexahydro-2H-pyrido[4,3-b][1,4]oxazin-3(4H)-one;
(4aR,8aS)-6-(4-((2-chloro-4-fluorophenoxy)methyl)-3-methylpiperidine-1-carbonyl)hexahydro-2H-pyrido[4,3-b][1,4]oxazin-3(4H)-one;
(4aR,8aS)-6-(3-((4-(difluoromethoxy)benzyl)oxy)azetidine-1-carbonyl)hexahydro-2H-pyrido[4,3-b][1,4]oxazin-3(4H)-one;
(4aR,8aS)-6-(3-((2-chloro-4-(trifluoromethyl)benzyl)oxy)cyclobutane-1-carbonyl)hexahydro-2H-pyrido[4,3-b][1,4]oxazin-3(4H)-one;
(4aR,8aS)-6-(3-((5-(trifluoromethyl)pyridin-2-yl)methoxy)azetidine-1-carbonyl)hexahydro-2H-pyrido[4,3-b][1,4]oxazin-3(4H)-one;
(4aR,8aS)-6-(3-((4-(trifluoromethoxy)benzyl)oxy)azetidine-1-carbonyl)hexahydro-2H-pyrido[4,3-b][1,4]oxazin-3(4H)-one;
N-(2-chloro-4-fluorophenyl)-1-((4aR,8aS)-3-oxooctahydro-2H-pyrido[4,3-b][1,4]oxazine-6-carbonyl)azetidine-3-carboxamide;
(4aS,8aR)-6-(3-(1-(2-chloro-4-(trifluoromethyl)phenoxy)ethyl)azetidine-1-carbonyl)hexahydro-2H-pyrido[4,3-b][1,4]oxazin-3(4H)-one;
(4aR,8aS)-6-(4-(3-(trifluoromethyl)pyridazin-4-yl)piperidine-1-carbonyl)hexahydro-2H-pyrido[4,3-b][1,4]oxazin-3(4H)-one;
(4aR,8aS)-6-[3-[5-(2,4-difluorophenyl)-4H-1,2,4-triazol-3-yl]azetidine-1-carbonyl]-4,4a,5,7,8,8a-hexahydropyrido[4,3-b][1,4]oxazin-3-one; and
(4aR,8aS)-6-[3-[[4-fluoro-2-(trifluoromethyl)phenyl]methoxy]azetidine-1-carbonyl]-4,4a,5,7,8,8a-hexahydropyrido[4,3-b][1,4]oxazin-3-one.
E40. The compound of formula (I) according to E39, or a pharmaceutically acceptable salt thereof, selected from:
(4aR,8aS)-6-[3-[4-(3,3-difluorocyclobutoxy)phenyl]azetidine-1-carbonyl]-4,4a,5,7,8,8a-hexahydropyrido[4,3-b][1,4]oxazin-3-one;
(4aR,8aS)-6-[3-(4-tetrahydropyran-4-ylphenyl)azetidine-1-carbonyl]-4,4a,5,7,8,8a-hexahydropyrido[4,3-b][1,4]oxazin-3-one;
(4aR,8aS)-6-[3-[4-(3,3-difluorocyclobutyl)phenyl]azetidine-1-carbonyl]-4,4a,5,7,8,8a-hexahydropyrido[4,3-b][1,4]oxazin-3-one;
(4aR,8aS)-6-[3-[4-(2,2,2-trifluoro-1,1-dimethyl-ethoxy)phenyl]azetidine-1-carbonyl]-4,4a,5,7,8,8a-hexahydropyrido[4,3-b][1,4]oxazin-3-one;
(4aR,8aS)-6-(3-(4-(cyclopropylmethoxy)phenyl)azetidine-1-carbonyl)hexahydro-2H-pyrido[4,3-b][1,4]oxazin-3(4H)-one;
(4aR,8aS)-6-(4-((2-chloro-4-(trifluoromethyl)benzyl)oxy)piperidine-1-carbonyl)hexahydro-2H-pyrido[4,3-b][1,4]oxazin-3(4H)-one;
(4aR,8aS)-6-(3-((4-(pentafluoro-16-sulfaneyl)benzyl)oxy)azetidine-1-carbonyl)hexahydro-2H-pyrido[4,3-b][1,4]oxazin-3(4H)-one;
(4aR,8aS)-6-[4-[[2,4-bis(trifluoromethyl)phenyl]methyl]piperidine-1-carbonyl]-4,4a,5,7,8,8a-hexahydropyrido[4,3-b][1,4]oxazin-3-one; and
(4aR,8aS)-6-(3-((4-(trifluoromethoxy)benzyl)oxy)azetidine-1-carbonyl)hexahydro-2H-pyrido[4,3-b][1,4]oxazin-3(4H)-one.

In a particular embodiment, the present invention provides pharmaceutically acceptable salts of the compounds according to formula (I) as described herein, especially hydrochloride salts.

In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶C, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization.

Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates.

Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text: AcOH=acetic acid, ACN=acetonitrile, Bn=benzyl, Boc=tert-butyloxycarbonyl, CAS RN=chemical abstracts registration number, Cbz=benzyloxycarbonyl, Cs₂CO₃=cesium carbonate, CO=carbon monoxide, CuCl=copper(I) chloride, CuCN=copper(I) cyanide, CuI=copper(I) iodide, DAST=(diethylamino)sulfur trifluoride, DBU=1,8-diazabicyclo[5,4,0]undec-7-ene, DEAD=diethyl azodicarboxylate, DIAD=diisopropyl azodicarboxylate, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC.HCl═N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-H-benzotriazole; HPLC=high performance liquid chromatography, iPrMgCl=isopropylmagnesium chloride, I₂=iodine, IPA=2-propanol, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, mCPBA=meta-chloroperoxybenzoic acid, MgSO₄=magnesium sulfate, min=minute(s), mL=milliliter, MPLC=medium pressure liquid chromatography, MS=mass spectrum, nBuLi=n-butyllithium, NaBH₃CN=sodium cyanoborohydride, NaH=sodium hydride, NaHCO₃=sodium hydrogen carbonate, NaNO₂=sodium nitrite, NaBH(OAc)₃=sodium triacetoxyborohydride, NaOH=sodium hydroxide, Na₂CO₃=sodium carbonate, Na₂SO₄=sodium sulfate, Na₂S₂O₃=sodium thiosulfate, NBS═N-bromosuccinimide, nBuLi=n-butyllithium, NEt₃=triethylamine (TEA), NH₄Cl=ammonium chloride, NMP=N-methyl-2-pyrrolidone, OAc=Acetoxy, T₃P=propylphosphonic anhydride, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(0), Pd(OAc)₂=palladium(II) acetate, Pd(OH)₂=palladium hydroxide, Pd(PPh₃)4=tetrakis(triphenylphosphine)palladium(0), PTSA=p-toluenesulfonic acid, R=any group, RT=room temperature, SFC=Supercritical Fluid Chromatography, S—PHOS=2-dicyclohexylphosphino-2′,6′-dimethoxybiphenyl, TBAI=tetra butyl ammonium iodine, TEA=triethylamine, TFA=trifluroacetic acid, THF=tetrahydrofuran, TMEDA=N,N,N′,N′-tetramethylethylenediamine, ZnCl₂=zinc chloride, Hal=halogen.

Compounds of formula IA wherein A, L¹, m, n and R¹ to R⁴ are as described herein and X is CH can be synthesized in analogy to literature procedures and/or as depicted for example in Scheme 1.

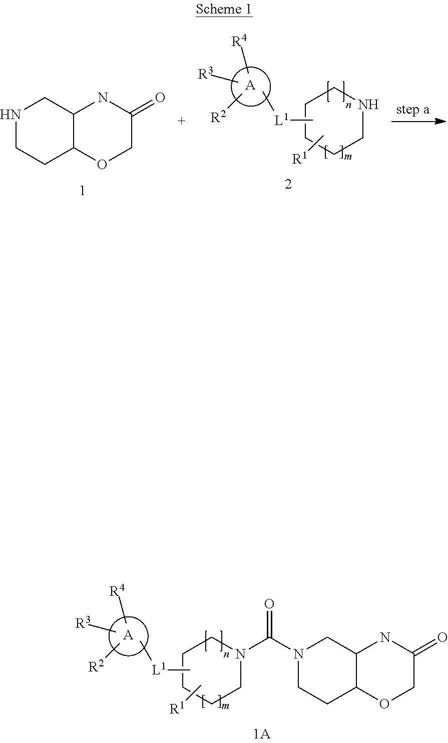

Accordingly, 4a,5,6,7,8,8a-hexahydro-4H-pyrido[4,3-b][1,4]oxazin-3-ones 1 are reacted with intermediates 2 in the presence of a urea forming reagent such as bis(trichloromethyl) carbonate using a suitable base and solvent such as, e.g. sodium bicarbonate in DCM, to give compounds of formula IA (step a). Further urea forming reagents include but are not limited to phosgene, trichloromethyl chloroformate, (4-nitrophenyl)carbonate, 1,1′-carbonyldiimidazole or 1,1′-carbonyl-di-(1,2,4-triazole). Reactions of this type and the use of these reagents are widely described in literature (e.g. G. Sartori et al., Green Chemistry 2000, 2, 140). A person skilled in the art will acknowledge that the order of the addition of the reagents can be important in this type of reactions due to the reactivity and stability of the intermediary formed carbamoyl chlorides, as well as for avoiding formation of undesired symmetrical urea by-products.

Compounds of formula IB wherein A, L¹, m, n and R¹ to R⁴ are as described herein and X is CH can be synthesized in analogy to literature procedures and/or as depicted for example in Scheme 2.

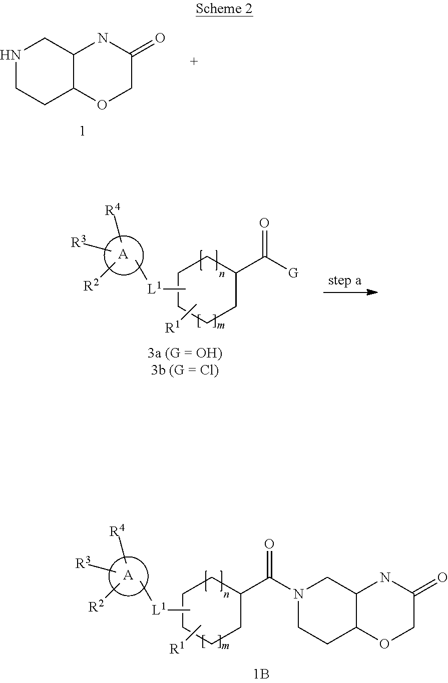

Accordingly, intermediates 1 can be coupled with an activated form of a carboxylic acid 3a (G=OH) or alternatively with carboxylic acid chlorides 3b (G=Cl) to provide compounds IB (step a). Amide couplings of this type are widely described in the literature and can be accomplished by the usage of coupling reagents such as CDI, DCC, HATU, HBTU, HOBT, TBTU, T3P or Mukaiyama reagent (Mukaiyama T. Angew. Chem., Int. Ed. Engl. 1979, 18, 707) in a suitable solvent e.g., DMF, DMA, DCM or dioxane, optionally in the presence of a base (e.g., TEA, DIPEA (Huenig's base) or DMAP).

Alternatively, the carboxylic acids 3a can be converted into their acid chlorides 3b by treatment with, e.g. thionyl chloride or oxalyl chloride, neat or optionally in a solvent such as DCM. Reaction of the acid chloride with intermediates 1 in an appropriate solvent such as DCM or DMF and a base, e.g. TEA, Huenig's base, pyridine, DMAP or lithium bis(trimethylsilyl)amide at temperatures ranging from 0° C. to the reflux temperature of the solvent or solvent mixture yields compounds IB (step a).

Intermediates 1 may be synthesized as depicted for example in Scheme 3 and/or in analogy to methods described in literature.

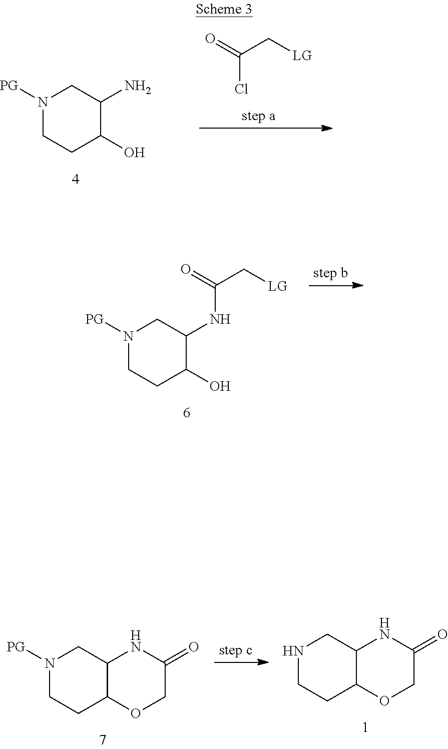

Thus, 3-aminopiperidin-4-ol derivatives 4 in which “PG” signifies a suitable protective group such as a Cbz or Boc protective group can be acylated for example with acyl chlorides 5 in which “LG” signifies a suitable leaving group (e.g., Cl or Br), using a suitable base such as sodium or potassium carbonate, sodium hydroxide or sodium acetate in an appropriate solvent such as THF, water, acetone or mixtures thereof, to provide intermediates 6 (step a).

Intermediates 4 are either commercially available or can be prepared according to literature methods in racemic or enantiomerically pure form.

Intermediates 6 can be cyclized to intermediates 7 using methods well known in the art, for example by treatment of 6 with sodium hydride in THF or potassium tert-butoxide in IPA and water (step b). Reactions of that type are described in literature (e.g. Z. Rafinski et al., J. Org. Chem. 2015, 80, 7468; S. Dugar et al., Synthesis 2015, 47(5), 712; WO2005/066187).

Removal of the protective group in intermediates 7, applying methods known in the art (e.g., a Boc group using TFA in DCM at temperatures between 0° C. and room temperature, a Cbz group using hydrogen in the presence of a suitable catalyst such as Pd or Pd(OH)₂ on charcoal in a suitable solvent such as MeOH, EtOH, EtOAc or mixtures thereof and as described for example in “Protective Groups in Organic Chemistry” by T.W. Greene and P.G.M. Wuts, 4th Ed., 2006, Wiley N.Y.), furnishes intermediates 1 (step c).

Intermediates 1 can be obtained as mixtures of diastereomers and enantiomers, respectively, or as single stereoisomers depending on whether racemic mixtures or enantiomerically pure forms of cis- or trans-3-aminopiperidin-4-ol derivatives 4 are employed in their syntheses. Intermediates 4 are commercially available and their synthesis has also been described in literature (e.g. WO2005/066187; WO2011/0059118; WO2016/185279).

Optically pure forms of intermediates 1 can be obtained for example by methods well known in the art from commercially available racemic forms of 4a,5,6,7,8,8a-hexahydro-4H-pyrido[4,3-b][1,4]oxazin-3-ones (1) (optionally in form of a salt such as, e.g. a hydrochloride salt) applying methods known in the art, e.g. by diastereomeric salt crystallization or by chiral chromatography.

In some embodiments, intermediates 2 are intermediates of type B. Intermediates of type B in which L¹ is CH₂ and X is N, and in which A, m, n and R¹ are as described herein can be prepared by methods well known by a person skilled in the art and as exemplified by the general synthetic procedures outlined in Scheme 4.

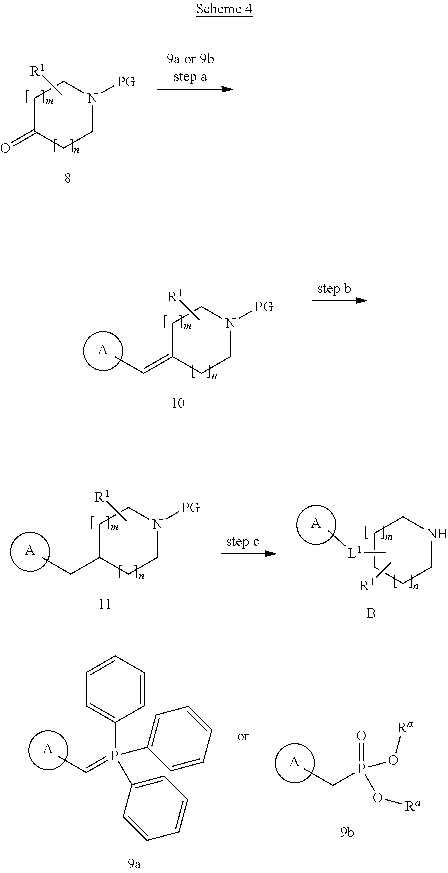

Ketones 8, either commercially available or prepared by methods known in the art, can be subjected for example to a Wittig reaction with alkylidene triphenylphosphoranes of type 9a in a suitable solvent such as, e.g. THF, Methyl-THF or DMSO to give intermediates 10 (step a). Phosphoranes 9a can be formed by treating the corresponding phosphonium salts with a suitable base such as BuLi, NaH, or KOtBu in a suitable solvent such as THF, dioxane or Methyl-THF and may be isolated or used in situ. Phosphonium salts in turn are readily available from an aryl/heteroaryl/heterocyclic-substituted alkylhalide (with halide being Cl, Br and iodo) and triphenylphosphine in a suitable solvent such as toluene. Heating may be applied to accelerate the reaction or drive the reaction to completion (e.g. H. J. Cristau, F. Plenat in PATAIS Chemistry of Functional Groups, Editor(s): Frank R. Hartley, 07th August 2006, Series Editor(s): Prof Saul Patai).

Alternatively, intermediates 10 can be obtained using a Horner-Wadsworth-Emmons (HWE) reaction using ketones 8 and phosphonates 9b, wherein Ra is alkyl, for example methyl or ethyl. Phosphonates 9b are in situ α-metalated using a suitable base and solvent such as NaH, nBuLi or KOtBu in THF (step a). Phosphonates 9b are readily prepared using for example the Arbuzov reaction by alkylation of an aryl/heteroaryl/heterocyclic halide (with halide being Cl, Br and iodo) with commercially available trialkyl phosphite (e.g. Chem. Rev. 1984, 84, 577).

Olefination reactions of both types are broadly described in literature (e.g. Current Org. Chem. 2015, 19(9), page 744; Chem. Rev. 1989, 89(4), 863; Org. React. 1977, 25, 73; Liebigs Ann./Recueil 1997, 1283; Acc. Chem. Res. 1983, 16, 411).

Reduction of the double bond in intermediates 10 using, e.g. hydrogen in the presence of a suitable catalyst such as palladium on charcoal in an appropriate solvent or solvent mixture such as EtOAc, MeOH or AcOH yields compounds 11 (step b).

Removal of the protective group from intermediates 11 applying methods known in the art (e.g., a Boc group using TFA in DCM or 4M HCl in dioxane at temperatures between 0° C. and room temperature, a Cbz group using hydrogen in the presence of a suitable catalyst such as Pd or Pd(OH)2 on charcoal in a suitable solvent such as MeOH, EtOH, EtOAc or mixtures thereof and as described for example in “Protective Groups in Organic Chemistry” by T.W. Greene and P.G.M. Wuts, 4th Ed., 2006, Wiley N.Y.), furnishes intermediates B (step c).

Alternatively, intermediates of type B in which L¹ is CH₂ and X is N, and in which A, m, n and R¹ are as described herein, can be prepared by methods well known in the art and as exemplified by the general synthetic procedures outlined in Scheme 5.

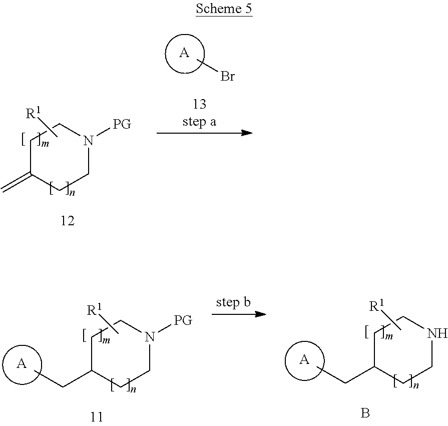

Commercially available alkene intermediates 12 in which PG signifies a suitable protecting group can treated with 9-Borabicyclo(3.3.1)nonane and then be subjected to Palladium-catalyzed Suzuki cross-coupling reactions with compounds 13, either commercially available or prepared by methods known in the art, yielding intermediate 11 (step a). Reactions of this type are broadly described in literature and are well known to persons skilled in the art.

Removal of the protective group from intermediates 11 applying literature methods and as described for example under Scheme 4, step c, furnishes intermediates B (step b).

In some embodiments, intermediates 2 are intermediates of type C. Intermediates of type C in which L¹ is —CHR^{L}O— and X is N, and in which A, m, n, R¹ are as described herein can be prepared by methods well known in the art and as exemplified by the general synthetic procedures outlined in Scheme 6.

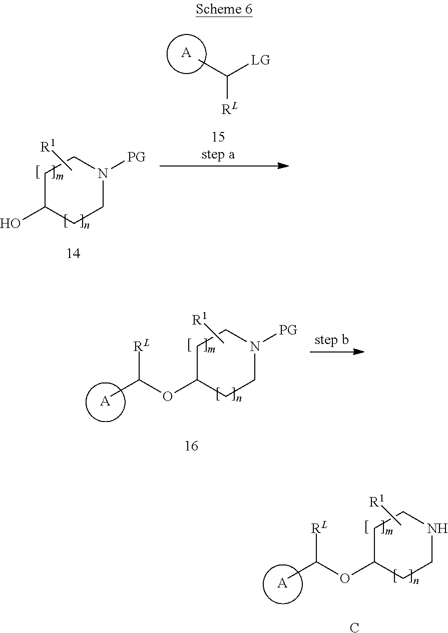

Intermediates 16 may be prepared from alcohols 14 in which PG is a suitable protective group such as a Cbz, Boc or Bn, that can be alkylated with compounds 15 in which LG is a suitable leaving group such as chlorine, bromine, iodine, OSO₂alkyl (e.g. methanesulfonate), OSO₂fluoroalkyl (e.g. trifluoromethanesulfonate) or OSO₂aryl (e.g. p-toluenesulfonate) using a suitable base, such as sodium hydride, potassium tert-butoxide, in an appropriate solvent (e.g. in DMF or THF) at temperatures between 0° C. and the boiling temperature of the solvent (step a).

Removal of the protective group from intermediates 16 applying literature methods and as described for example under Scheme 4, step c, furnishes intermediates C (step b).

In some embodiments, intermediates 2 are intermediates of type D. Intermediates of type D in which Lt is —OCHR^{L}— and X is N, and in which A, m, n, R¹ are as described herein, can be prepared by methods well known in the art and as exemplified by the general synthetic procedures outlined in Scheme 7.

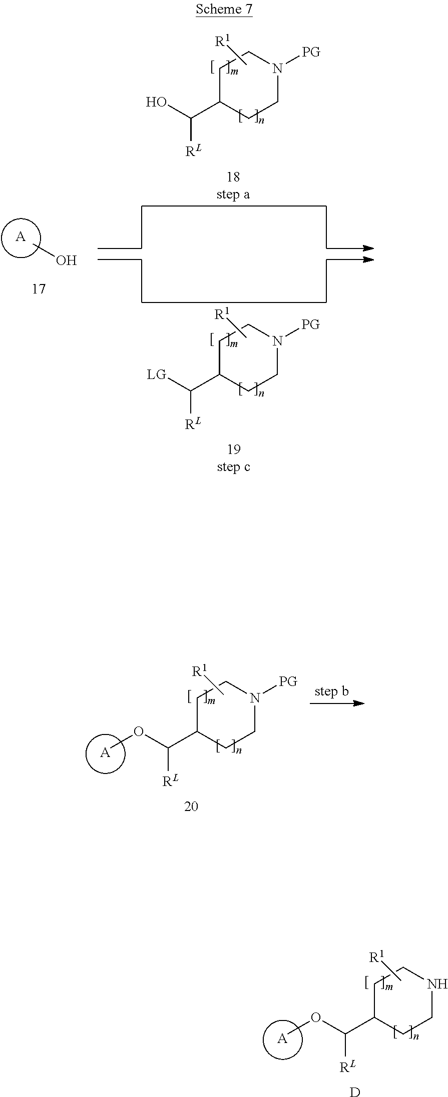

Alcohols of type 17 can be subjected to a Mitsunobu reaction with intermediates 18 in which PG is a suitable protective group such as a Cbz, Boc or Bn, using an appropriate phosphine such as triphenylphosphine and a dialkyl azodicarboxylate such as DEAD or DIAD in a suitable solvent such as THF to give intermediates 20 (step a). Mitsunobu reactions of that type are broadly described in literature (e.g. Org. Chem. Front. 2015, 2, 739; Chem. Rev. 2009, 109 (6), 2551).

Removal of the protective group from intermediates 20 applying literature methods and as described for example under Scheme 4, step c, furnishes intermediates D (step b). Alternatively, intermediates 20 may be prepared from alcohols 17 that can be alkylated with compounds 19 in which LG is a suitable leaving group such as chlorine, bromine, iodine, OSO₂alkyl (e.g. methanesulfonate), OSO₂fluoroalkyl (e.g. trifluoromethanesulfonate) or OSO₂aryl (e.g. p-toluenesulfonate) using a suitable base such as Cs₂CO₃, NaH, in an appropriate solvent, such as DMF at temperatures between 0° C. and the boiling temperature of the solvent (step c).

In some embodiments, intermediates 2 are intermediates of type E. Intermediates of type E, in which L¹ is a covalent bond and X is N, and in which A, m, n, R¹ are as described herein. Intermediates of that type can be prepared by methods well known in the art and as exemplified by the general synthetic procedures outlined in Scheme 8.

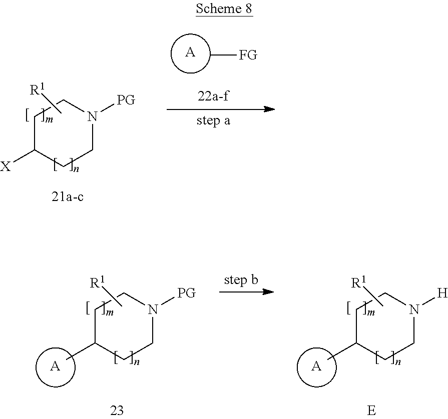

Intermediates 21a in which PG signifies a suitable protecting group and X is a boronic acid (FG=B(OH)₂) or a boronic ester (FG=e.g. 4,4,5,5-tetramethyl-2-phenyl-1,3,2-dioxaborolane (pinacol) ester) are either commercially available or prepared using literature procedures as described for example in “Boronic Acids—Preparation and Applications in Organic Synthesis and Medicine” by Dennis G. Hall (ed.) 1st Ed., 2005, John Wiley & Sons, New York). Intermediates 21a can be subjected to cross-coupling reactions such as Suzuki coupling reactions with compounds 22a, which are either commercially available or prepared by methods known in the art, in which FG signifies a suitable functional group such as, e.g. chloro, bromo, iodo, —OSO₂fluoroalkyl (e.g. triflate (trifluoromethanesulfonate), using a suitable catalyst (e.g. dichloro[1,1′-bis(diphenylphosphino)-ferrocene]palladium(II) dichloromethane adduct, tetrakis(triphenylphosphine)palladium(0) or palladium(II)acetate with triphenylphosphine) in an appropriate solvent (e.g. dioxane, dimethoxyethane, water, toluene, DMF or mixtures thereof) and a suitable base (e.g. Na₂CO₃, NaHCO₃, KF, K₂CO₃ or TEA) at temperatures between room temperature and the boiling point of the solvent or solvent mixture, to yield intermediates 23 (step a). Suzuki reactions of this type are broadly described in literature (e.g. A. Suzuki, Pure Appl. Chem. 1991, 63, 419-422; A. Suzuki, N. Miyaura, Chem. Rev. 1995, 95, 2457-2483; A. Suzuki, J. Organomet. Chem. 1999, 576, 147-168; V. Polshettiwar et al., Chem. Sus. Chem. 2010, 3, 502-522) and are well known to those skilled in the art.

Alternatively, aryl- or heteroaryl-trifluoroborates 21b (FG=BF₃K) can be used in the cross-coupling reaction with 22a applying a palladium catalyst such as, e.g. tetrakis(triphenylphosphine)-palladium(0), palladium(II) acetate or dichloro[1,1′-bis(diphenylphosphino)ferrocene]-palladium(II) dichloromethane adduct in the presence of a suitable base such as cesium carbonate or potassium phosphate in solvents such as toluene, THF, dioxane, water or mixtures thereof, at temperatures between room temperature and the boiling point of the solvent or solvent mixture.

Alternatively, intermediates 21c, in which X is bromide or Iodide, can be reacted with aryl or heteroaryl stannanes 22b in which FG is Sn(alkyl)₃ and alkyl is preferable n-butyl or methyl, using a suitable catalyst and solvent such as, e.g. tetrakis(triphenylphosphine)-palladium(0) in DMF at temperatures between room temperature and the boiling point of the solvent or solvent mixture to provide intermediates 23 (step a). Stille reactions of that type are well known in the art and described in literature, e.g. Org. React. 1997, 50, 1-652, ACS Catal. 2015, 5, 3040-3053.

Furthermore, intermediates 21c, in which X is bromide or iodide, can be reacted with aryl or heteroarylzinc halides 22c in which FG is ZnHal and Hal preferably bromide or iodide, either commercially available or prepared by literature methods, using an appropriate catalyst and solvent system such as, e.g. [1,1′-bis(diphenylphosphino)ferrocene]dichloropalladium(II) and copper(I)iodide in DMA, or tetrakis(triphenylphosphine)palladium(0) in THF or DMF at temperatures between room temperature and the boiling point of the solvent to provide intermediates 23. (step a). Negishi reactions of that type are well known in the art and also described in literature, e.g. Org. Lett., 2005, 7, 4871, ACS Catal. 2016, 6 (3), 1540-1552. Acc. Chem. Res. 1982, 15 (11), pp 340-348. Alternatively, intermediates 23 may be prepared by converting intermediates 21c in which X is for example iodide into the corresponding zinc species by applying literature methods (e.g. reaction of 21c with Zn powder in the presence of chlorotrimethylsilane and 1,2-dibromoethane in a suitable solvent such as DMA) and coupling of the zinc species with aryl- or heteroarylbromides- or iodides 22a under the conditions mentioned before.

Alternatively, intermediates 21a in which X is preferably bromide can be subjected to a cross-electrophile coupling with aryl- or heteroarylbromides 22a in which FG signifies bromide under irradiation with a 420 nm blue light lamp using an appropriate photo catalyst such as [Ir{dF(CF₃)ppy}2(dtbpy)]PF₆ ([4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate), a Nickel catalyst like NiCl₂ glyme (dichloro(dimethoxyethane)nickel), 4,4′-di-tert-butyl-2,2′-dipyridyl and tris(trimethylsilyl)silane, in the presence of a suitable base such as anhydrous sodium carbonate in a solvent like DME. Reactions of this type are described in literature, e.g. J. Am. Chem. Soc. 2016, 138, 8084. (step a).

Alternatively, intermediates 21b in which X is an trifluoroborate (X═BF₃K) can used to directly alkylate unfunctionalized heteroaryls 22d in which FG is a hydrogen in an adapted Minisci-type coupling. Minisci reactions of this kind require an oxidant such as Mn(OAc)₃ and acid such as TFA. Minisci reactions of this type are described in literature (e.g. Molander et al, Org. Lett. 2011, Vol. 13, No. 7, 1852-1855) and are well known to those skilled in the art.

Removal of the protective group from intermediates 23 applying methods well known in the art and as described for example under Scheme 4, step c, furnishes intermediates E (step b).

In some embodiment, intermediates 2 are intermediates of type F. Intermediates of type F in which L¹ is an amide bond —NHC(O)— and X is N, and in which A, m, n, R¹ are as described herein can be prepared by methods well known by a person skilled in the art and as exemplified by the general synthetic procedure outlined in Scheme 9.

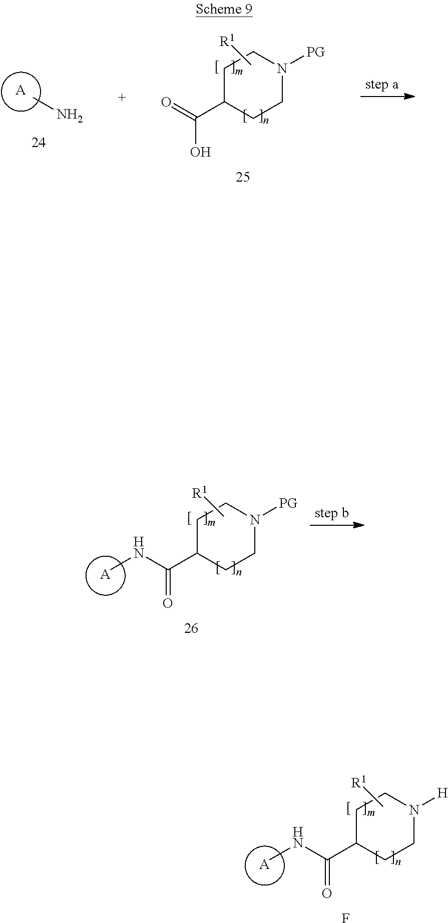

Carboxylates 25, either commercially available or prepared by methods known in the art, and in which PG signifies a suitable protecting group such as, e.g. a Boc, Cbz or Bn protecting group, can be subjected to an amide coupling with amines 24, using a suitable coupling reagent, such as HATU, HBTU, DCC, EDC, preferably HATU and an appropriate base such as, e.g., DIPEA and suitable solvent system such as, e.g. DMF, NMP, CH₃CN or DCM, preferably DMF and in a temperature range between room temperature and 100° C., preferably around room temperature to give intermediates 26 (step a).

Removal of the protective group from intermediates 26 applying methods known in the art or as described under Scheme 4, step c, furnishes intermediates F (step b).

In some embodiments, intermediates 3 are intermediates of type G. Intermediates of type G in which L¹ is —CHR^{L}O— and X is CH, and in which A, m, n, R¹ are as described herein can be prepared by methods well known in the art and as exemplified by the general synthetic procedures outlined in Scheme 10.

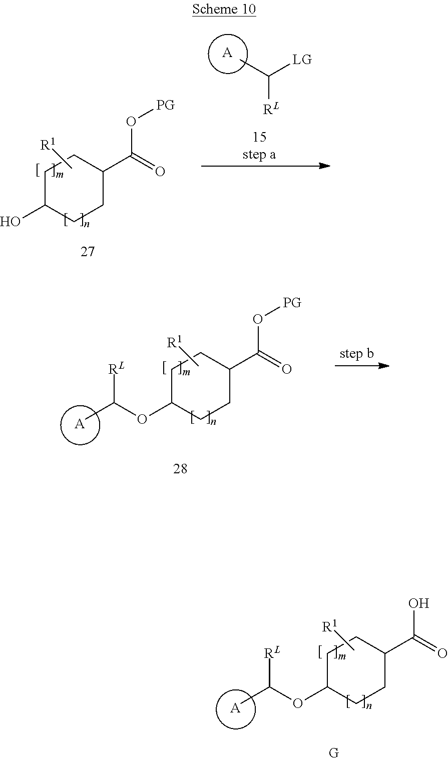

Intermediates 28 may be prepared from alcohols 27 in which PG is a suitable protective group such as a methyl or tBu-ester, that can be alkylated with compounds 15 in which LG is a suitable leaving group such as chlorine, bromine, iodine, OSO₂alkyl (e.g. methanesulfonate), OSO₂fluoroalkyl (e.g. trifluoromethanesulfonate) or OSO₂aryl (e.g. p-toluenesulfonate) using a suitable base, such as sodium hydride, potassium tert-butoxide, in an appropriate solvent (e.g. in DMF or THF) at temperatures between 0° C. and the boiling temperature of the solvent (step a).

Removal of the protective group from intermediates 28 by applying literature methods, for example with LiOH in THF/water at RT in the case of an methyl ester, furnishes intermediates G (step b).

A person skilled in the art will appreciate that the processes for making intermediates of type B-G outlined in Schemes 4-9 is also applicable for making intermediates of type 3a,b (see Scheme 2).

In one aspect, the present invention provides a process of manufacturing the compounds of formula (I) as described herein, comprising:
(a) reacting a first amine 4a,5,6,7,8,8a-hexahydro-4H-pyrido[4,3-b][1,4]oxazin-3-one (1),

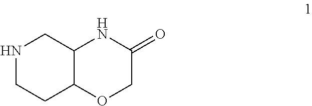 [SMILES: O=C1COC2CCNCC2N1]

with a second amine 2, wherein A, L¹, m, n, and R¹ to R⁴ are as defined herein

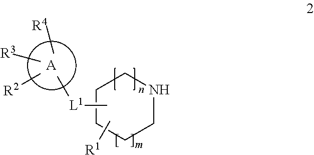

in the presence of a base and a urea forming reagent, to form a compound of formula (IA), wherein A, L¹, m, n, and R¹ to R⁴ are as defined herein,

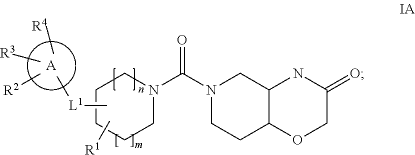

or
(b) reacting 4a,5,6,7,8,8a-hexahydro-4H-pyrido[4,3-b][1,4]oxazin-3-one (1),

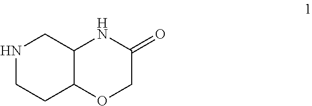 [SMILES: O=C1COC2CCNCC2N1]

with a carboxylic acid 3a, wherein A, L¹, m, n, and R¹ to R⁴ are as defined herein,

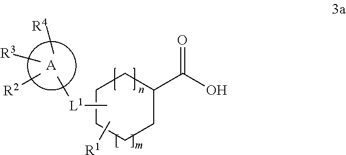

in the presence of a coupling reagent, such as CDI, DCC, HATU, HBTU, HOBT, TBTU, T3P or Mukaiyama reagent and optionally a base, such as TEA, DIPEA or DMAP, to form a compound of formula (IB), wherein A, L¹, m, n, and R¹ to R⁴ are as defined herein,

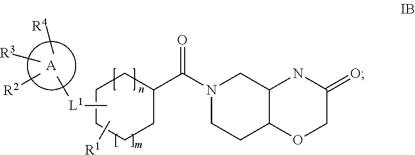

or
(c) reacting 4a,5,6,7,8,8a-hexahydro-4H-pyrido[4,3-b][1,4]oxazin-3-one (1),

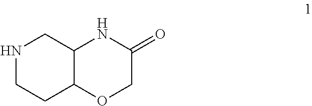 [SMILES: O=C1COC2CCNCC2N1]

with a carboxylic acid chloride 3b, wherein A, L¹, m, n, and R¹ to R⁴ are as defined herein,

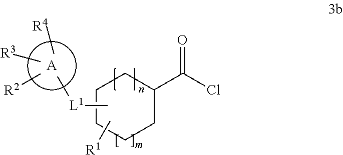

in the presence of a base, e.g. TEA, Huenig's base, pyridine, DMAP or lithium bis(trimethylsilyl)amide, to form a compound of formula (IB), wherein A, L¹, m, n, and R¹ to R⁴ are as defined herein,

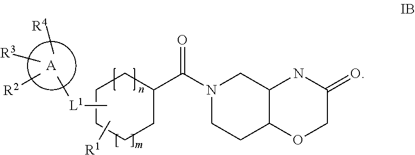

In one embodiment, there is provided a process according to the invention, wherein said base of option (a) is sodium bicarbonate.

In one embodiment, there is provided a process according to the invention, wherein said urea forming reagent of option (a) is selected from bis(trichloromethyl) carbonate, phosgene, trichloromethyl chloroformate, (4-nitrophenyl)carbonate and 1,1′-carbonyldiimidazole, preferably wherein said urea forming reagent is bis(trichloromethyl) carbonate.

In one aspect, the present invention provides a compound of formula (I) as described herein, when manufactured according to any one of the processes described herein.

MAGL Inhibitory Activity

Compounds of the present invention are MAGL inhibitors. Thus, in one aspect, the present invention provides the use of compounds of formula (I) as described herein for inhibiting MAGL in a mammal.

In a further aspect, the present invention provides compounds of formula (I) as described herein for use in a method of inhibiting MAGL in a mammal.

In a further aspect, the present invention provides the use of compounds of formula (I) as described herein for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds were profiled for MAGL inhibitory activity by determining the enzymatic activity by following the hydrolysis of the natural substrate 2-arachidonoylglycerol (2-AG) resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 μM. 0.25 μL compound dilutions (100% DMSO) were added to 9 μL MAGL in assay buffer (50 mM TRIS (GIBCO, 15567-027), 1 mM EDTA (Fluka, 03690-100 mL), 0.01% (v/v) Tween. After shaking, the plate was incubated for 15 min at RT. To start the reaction, 10 μL 2-arachidonoylglycerol in assay buffer was added. The final concentrations in the assay was 50 μM MAGL and 8 μM 2-arachidonoylglyerol. After shaking and 30 min incubation at RT, the reaction was quenched by the addition of 40 μL of ACN containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an ACN/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| Example | IC₅₀ MAGL [nM] |
|---|---|
| 1 | 33.1 |
| 2 | 42 |
| 3 | 4.1 |
| 4 | 7.1 |
| 5 | 6 |
| 6 | 2.9 |
| 7 | 9.1 |
| 8 | 38.8 |
| 9 | 23.5 |
| 10 | 6 |
| 11 | 1.5 |
| 12 | 0.3 |
| 13 | 0.6 |
| 14 | 26.4 |
| 15 | 8.7 |
| 16 | 24.5 |
| 17 | 168 |
| 18 | 39.2 |
| 19 | 44.1 |
| 20 | 147.4 |
| 21 | 27.8 |
| 22 | 13.4 |
| 23 | 118.9 |
| 24 | 62.1 |
| 25 | 72.5 |
| 26 | 5.2 |
| 27 | 18.8 |
| 28 | 341.6 |
| 29 | 406.3 |
| 30 | 77.3 |
| 31 | 5.8 |
| 32 | 22.5 |
| 33 | 5.9 |
| 34 | 8 |
| 35 | 4.6 |
| 36 | 9.4 |
| 37 | 31.7 |
| 38 | 6.4 |
| 39 | 1.9 |
| 40 | 46.9 |
| 41 | 773.7 |
| 42 | 261.4 |
| 43 | 158.7 |
| 44 | 23.6 |
| 45 | 1249.1 |
| 46 | 224.8 |
| 47 | 90.9 |
| 48 | 333.1 |
| 49 | 34.0 |

In one aspect, the present invention provides compounds of formula (I) and their pharmaceutically acceptable salts or esters as described herein, wherein said compounds of formula (I) and their pharmaceutically acceptable salts or esters have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts or esters as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

Using the Compounds of the Invention

In one aspect, the present invention provides compounds of formula (I) as described herein, or pharmaceutically acceptable salts thereof, for use as therapeutically active substance.

In a further aspect, the present invention provides the use of compounds of formula (I) as described herein, or pharmaceutically acceptable salts thereof, for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein, or pharmaceutically acceptable salts thereof, for the treatment or prophylaxis of neuroinflammation and/or neurodegenerative diseases in a mammal.

In one embodiment, the present invention provides the use of compounds of formula (I) as described herein, or pharmaceutically acceptable salts thereof, for the treatment or prophylaxis of neurodegenerative diseases in a mammal.

In one embodiment, the present invention provides the use of compounds of formula (I) as described herein, or pharmaceutically acceptable salts thereof, for the treatment or prophylaxis of cancer in a mammal.

In one embodiment, the present invention provides the use of compounds of formula (I) as described herein, or pharmaceutically acceptable salts thereof, for the treatment or prophylaxis of inflammatory bowel disease in a mammal.

In one embodiment, the present invention provides the use of compounds of formula (I) as described herein, or pharmaceutically acceptable salts thereof, for the treatment or prophylaxis of pain in a mammal.

In one aspect, the present invention provides the use of compounds of formula (I) as described herein, or pharmaceutically acceptable salts thereof, for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein, or pharmaceutically acceptable salts thereof, for the treatment or prophylaxis of multiple sclerosis, Alzheimer's disease and/or Parkinson's disease in a mammal.

In a particularly preferred embodiment, the present invention provides the use of compounds of formula (I) as described herein, or pharmaceutically acceptable salts thereof, for the treatment or prophylaxis of multiple sclerosis in a mammal.

In one aspect, the present invention provides compounds of formula (I) as described herein, or pharmaceutically acceptable salts thereof, for use in the treatment or prophylaxis of neuroinflammation, neurodegenerative diseases, pain, cancer, mental disorders and/or inflammatory bowel disease in a mammal.

In one embodiment, the present invention provides compounds of formula (I) as described herein, or pharmaceutically acceptable salts thereof, for use in the treatment or prophylaxis of neuroinflammation and/or neurodegenerative diseases in a mammal.

In one embodiment, the present invention provides compounds of formula (I) as described herein, or pharmaceutically acceptable salts thereof, for use in the treatment or prophylaxis of cancer in a mammal.

In one embodiment, the present invention provides compounds of formula (I) as described herein, or pharmaceutically acceptable salts thereof, for use in the treatment or prophylaxis of neurodegenerative diseases in a mammal.

In one embodiment, the present invention provides compounds of formula (I) as described herein, or pharmaceutically acceptable salts thereof, for use in the treatment or prophylaxis of inflammatory bowel disease in a mammal.

In one embodiment, the present invention provides compounds of formula (I) as described herein, or pharmaceutically acceptable salts thereof, for use in the treatment or prophylaxis of pain in a mammal.

In one aspect, the present invention provides compounds of formula (I) as described herein, or pharmaceutically acceptable salts thereof, for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides compounds of formula (I) as described herein, or pharmaceutically acceptable salts thereof, for use in the treatment or prophylaxis of multiple sclerosis, Alzheimer's disease and/or Parkinson's disease in a mammal.

In a particularly preferred embodiment, the present invention provides compounds of formula (I) as described herein, or pharmaceutically acceptable salts thereof, for use in the treatment or prophylaxis of multiple sclerosis in a mammal.

In one aspect, the present invention provides the use of compounds of formula (I) as described herein, or pharmaceutically acceptable salts thereof, for the preparation of a medicament for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein, or pharmaceutically acceptable salts thereof, for the preparation of a medicament for the treatment or prophylaxis of neuroinflammation and/or neurodegenerative diseases in a mammal.

In one embodiment, the present invention provides the use of compounds of formula (I) as described herein, or pharmaceutically acceptable salts thereof, for the preparation of a medicament for the treatment or prophylaxis of neurodegenerative diseases in a mammal.

In one embodiment, the present invention provides the use of compounds of formula (I) as described herein, or pharmaceutically acceptable salts thereof, for the preparation of a medicament for the treatment or prophylaxis of cancer in a mammal.

In one embodiment, the present invention provides the use of compounds of formula (I) as described herein, or pharmaceutically acceptable salts thereof, for the preparation of a medicament for the treatment or prophylaxis of inflammatory bowel disease in a mammal.

In one embodiment, the present invention provides the use of compounds of formula (I) as described herein, or pharmaceutically acceptable salts thereof, for the preparation of a medicament for the treatment or prophylaxis of pain in a mammal.

In a further aspect, the present invention provides the use of compounds of formula (I) as described herein, or pharmaceutically acceptable salts thereof, for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein, or pharmaceutically acceptable salts thereof, for the preparation of a medicament for the treatment or prophylaxis of multiple sclerosis, Alzheimer's disease and/or Parkinson's disease in a mammal.

In a particularly preferred embodiment, the present invention provides the use of compounds of formula (I) as described herein, or pharmaceutically acceptable salts thereof, for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer, mental disorders and/or inflammatory bowel disease in a mammal, which method comprises administering an effective amount of a compound of formula (I) as described herein, or a pharmaceutically acceptable salt thereof, to the mammal.

In one embodiment, the present invention provides a method for the treatment or prophylaxis of neuroinflammation and/or neurodegenerative diseases in a mammal, which method comprises administering an effective amount of a compound of formula (I) as described herein, or a pharmaceutically acceptable salt thereof, to the mammal.

In one embodiment, the present invention provides a method for the treatment or prophylaxis of neurodegenerative diseases in a mammal, which method comprises administering an effective amount of a compound of formula (I) as described herein, or a pharmaceutically acceptable salt thereof, to the mammal.

In one embodiment, the present invention provides a method for the treatment or prophylaxis of cancer in a mammal, which method comprises administering an effective amount of a compound of formula (I) as described herein, or a pharmaceutically acceptable salt thereof, to the mammal.

In one embodiment, the present invention provides a method for the treatment or prophylaxis of inflammatory bowel disease in a mammal, which method comprises administering an effective amount of a compound of formula (I) as described herein, or a pharmaceutically acceptable salt thereof, to the mammal.

In one embodiment, the present invention provides a method for the treatment or prophylaxis of pain in a mammal, which method comprises administering an effective amount of a compound of formula (I) as described herein, or a pharmaceutically acceptable salt thereof, to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal, which method comprises administering an effective amount of a compound of formula (I) as described herein, or a pharmaceutically acceptable salt thereof, to the mammal.

In a preferred embodiment, the present invention provides a method for the treatment or prophylaxis of multiple sclerosis, Alzheimer's disease and/or Parkinson's disease in a mammal, which method comprises administering an effective amount of a compound of formula (I) as described, or a pharmaceutically acceptable salt thereof, herein to the mammal.

In a particularly preferred embodiment, the present invention provides a method for the treatment or prophylaxis of multiple sclerosis in a mammal, which method comprises administering an effective amount of a compound of formula (I) as described herein, or a pharmaceutically acceptable salt thereof, to the mammal.

Pharmaceutical Compositions and Administration

In one aspect, the present invention provides a pharmaceutical composition comprising a compound of formula (I) as described herein and a therapeutically inert carrier.

Exemplary, non-limiting examples of pharmaceutical compositions according to the invention are described in Examples 50 and 51.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### (4aR,8aS)-6-[3-[4-[(3R or S)-Tetrahydrofuran-3-yl]phenyl]azetidine-1-carbonyl]-4,4a,5,7,8,8a-hexahydropyrido[4,3-b][1,4]oxazin-3-one

### and

### Example 2

### (4aR,8aS)-6-[3-[4-[(3S or R)-Tetrahydrofuran-3-yl]phenyl]azetidine-1-carbonyl]-4,4a,5,7,8,8a-hexahydropyrido[4,3-b][1,4]oxazin-3-one

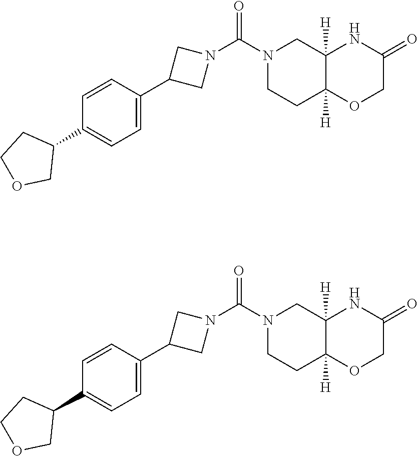 [SMILES: O=C1CO[C@H]2CCN(C(=O)N3CC(c4ccc([C@H]5CCOC5)cc4)C3)C[C@H]2N1 ; O=C1CO[C@H]2CCN(C(=O)N3CC(c4ccc([C@@H]5CCOC5)cc4)C3)C[C@H]2N1]

To a stirred solution at 60° C. of 3-(4-tetrahydrofuran-3-ylphenyl)azetidine; 2,2,2-trifluoroacetic acid (80 mg, 0.250 mmol) and (4-nitrophenyl) (4aR,8aS)-3-oxo-4,4a,5,7,8,8a-hexahydropyrido[4,3-b][1,4]oxazine-6-carboxylate (81 mg, 0.250 mmol) in ACN (5.04 mL) was added N,N-diisopropylethylamine (0.09 mL, 0.500 mmol, 2 eq). The solution was stirred at 60° C. for 16 h, then it was concentrated under vacuum to give a residue, which was purified by Prep-HPLC (TFA condition) to give (4aR,8aS)-6-[3-(4-tetrahydrofuran-3-ylphenyl)azetidine-1-carbonyl]-4,4a,5,7,8,8a-hexahydropyrido[4,3-b][1,4]oxazin-3-one (82 mg, 82.69%) as a white solid. This material was separed by chiral SFC into the two diastereoisomers to give (4aR,8aS)-6-[3-[4-[(3R or S)-tetrahydrofuran-3-yl]phenyl]azetidine-1-carbonyl]-4,4a,5,7,8,8a-hexahydropyrido[4,3-b][1,4]oxazin-3-one (Example 1, 35 mg, 40%; white solid; MS (ESI): m/z=386.1 [M+H]⁺) and (4aR,8aS)-6-[3-[4-[(3S or R)-tetrahydrofuran-3-yl]phenyl]azetidine-1-carbonyl]-4,4a,5,7,8,8a-hexahydropyrido[4,3-b][1,4]oxazin-3-one (Example 2, 33 mg, 39%; white solid; MS (ESI): m/z=386.1 [M+H]⁺).

### Step a) tert-butyl 3-(4-tetrahydrofuran-3-ylphenyl)azetidine-1-carboxylate

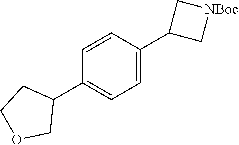 [SMILES: CC(C)(C)OC(=O)N1CC(c2ccc(C3CCOC3)cc2)C1]

This material was prepared in analogy to example 6, step a) starting from tert-butyl 3-(4-bromophenyl)azetidine-1-carboxylate (600 mg, 1.92 mmol) and 3-bromotetrahydrofuran (377 mg, 2.5 mmol). 340 mg (58%), light yellow solid. MS (ESI): m/z=248.0 [M-56+H]⁺.

### Step b) 3-(4-tetrahydrofuran-3-ylphenyl)azetidine; 2,2,2-trifluoroacetic acid

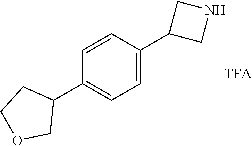 [SMILES: c1cc(C2CCOC2)ccc1C1CNC1]

A solution of tert-butyl 3-(4-tetrahydrofuran-3-ylphenyl)azetidine-1-carboxylate (80 mg, 0.260 mmol) and trifluoroacetic acid (0.3 mL, 3.89 mmol) in DCM (3 mL) was stirred at 20° C. for 4 h. The solution was concentrated in vacuo to give 3-(4-tetrahydrofuran-3-ylphenyl)azetidine; 2,2,2-trifluoroacetic acid (80 mg, 96%) as light yellow oil. MS (ESI): m/z=204.0 [M+H]⁺.

### Example 3

### (4aR,8aS)-6-[3-[4-(3,3-Difluorocyclobutoxy)phenyl]azetidine-1-carbonyl]-4,4a,5,7,8,8a-hexahydropyrido[4,3-b][1,4]oxazin-3-one

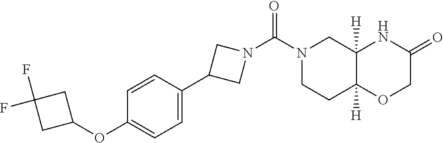 [SMILES: O=C1CO[C@H]2CCN(C(=O)N3CC(c4ccc(OC5CC(F)(F)C5)cc4)C3)C[C@H]2N1]

A mixture of (4-nitrophenyl) (4aR,8aS)-3-oxo-4,4a,5,7,8,8a-hexahydropyrido[4,3-b][1,4]oxazine-6-carboxylate (40.0 mg, 0.120 mmol), DIEA (146.2 mg, 1.13 mmol) and 3-[4-(3,3-difluorocyclobutoxy)phenyl]azetidine 2,2,2-trifluoroacetic acid salt (40.0 mg, 0.110 mmol) in ACN (2 mL) was stirred at 80° C. for 12 h. The mixture was concentrated and the residue purified by prep-HPLC (0.225% v/v FA in water and ACN) to give the title compound as white solid (6.6 mg, 13.2%). MS (ESI): m/z=422.3 [M+H]⁺.

### Step a) tert-Butyl 3-(4-hydroxyphenyl)azetidine-1-carboxylate

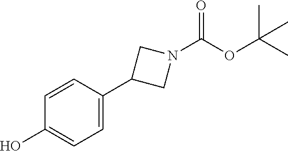 [SMILES: CC(C)(C)OC(=O)N1CC(c2ccc(O)cc2)C1]

To a 40 mL vial equipped with a stirring bar was added 4-bromophenol (1465.5 mg, 8.47 mmol), tert-butyl 3-bromoazetidine-1-carboxylate (2000.0 mg, 8.47 mmol, CAS RN 1064194-10-0), Ir[dF(CF₃)ppy]₂(dtbbpy)PF₆ (95.0 mg, 0.080 mmol), NiCl₂-glyme (9.31 mg, 0.040 mmol), 4-tert-butyl-2-(4-tert-butyl-2-pyridyl)pyridine (13.64 mg, 0.050 mmol), bis(trimethylsilyl)silyl-trimethyl-silane (2106.3 mg, 8.47 mmol) and Na₂CO₃ (1795.6 mg, 16.94 mmol) in DME (111 mL). The vial was sealed and placed under nitrogen. The reaction was stirred and irradiated with a 34 W blue LED lamp (7 cm distance), with cooling fan to keep the reaction temperature at 25° C., for 14 h. The reaction mixture was filtered and the filtrate was purified by reversed flash chromatography (0.1% v/v FA in water and ACN) to give the crude product, which was purified with silica gel column chromatography (PE:EtOAc=1:1) to give the desired compound as an off-white solid (1169 mg, 4.69 mmol, 55.4%). MS (ESI): m/z=194.0 [M-56+H]⁺.

### Step b) tert-Butyl 3-[4-(3,3-difluorocyclobutoxy)phenyl]azetidine-1-carboxylate

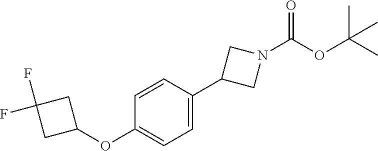 [SMILES: CC(C)(C)OC(=O)N1CC(c2ccc(OC3CC(F)(F)C3)cc2)C1]

A solution of PPh₃ (121.33 mg, 0.460 mmol) and DEAD (0.07 mL, 0.460 mmol) in toluene (5 mL) was stirred at 0° C. for 10 min, then 3,3-difluorocyclobutanol (50.0 mg, 0.460 mmol) was added. The reaction mixture was allowed to warm to 25° C., then tert-butyl 3-(4-hydroxyphenyl)azetidine-1-carboxylate (103.8 mg, 0.420 mmol) was added and the reaction mixture was stirred at 100° C. for 12 h. The reaction mixture was poured into H₂O (10 mL), extracted three times with EtOAc (10 mL each), the organic layers dried over Na₂SO₄, filtered and evaporated. The residue was purified with reversed-phase column chromatography (0.1% v/v FA in water and ACN) to give the title compound as light yellow solid (40 mg, 25.5%). MS (ESI): m/z=284.1 [M+H]⁺.

### Step c) 3-[4-(3,3-Difluorocyclobutoxy)phenyl]azetidine 2,2,2-trifluoroacetic Acid Salt

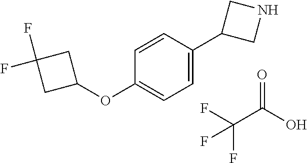

To a solution of tert-butyl 3-[4-(3,3-difluorocyclobutoxy)phenyl]azetidine-1-carboxylate (40.0 mg, 0.120 mmol) in DCM (2 mL) was added TFA (0.5 mL) and the mixture stirred at 20° C. for 12 h. The reaction mixture was evaporated under reduced pressure to give the desired compound as a yellow oil (40 mg, 0.110 mmol, 96.1%). MS (ESI): m/z=240.0 [M-56+H]⁺.

### Example 4

### (4aR,8aS)-6-[3-(4-Isopropoxyphenyl)azetidine-1-carbonyl]-4,4a,5,7,8,8a-hexahydropyrido[4,3-b][1,4]oxazin-3-one

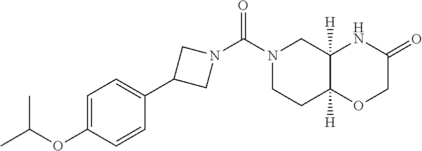 [SMILES: CC(C)Oc1ccc(C2CN(C(=O)N3CC[C@@H]4OCC(=O)N[C@@H]4C3)C2)cc1]

To a stirred solution at 60° C. of 3-(4-isopropoxyphenyl)azetidine hydrochloride (70 mg, 0.31 mmol) and (4-nitrophenyl) (4aR,8aS)-3-oxo-4,4a,5,7,8,8a-hexahydropyrido[4,3-b][1,4]oxazine-6-carboxylate (99 mg, 0.31 mmol) in ACN (5 mL) was added N,N-diisopropylethylamine (0.11 mL, 0.61 mmol). The solution was stirred at 60° C. for 16 h, then it was concentrated under vacuum to give a residue, which was purified by Prep-HPLC (TFA condition) to give (4aR,8aS)-6-[3-(4-isopropoxyphenyl)azetidine-1-carbonyl]-4,4a,5,7,8,8a-hexahydropyrido[4,3-b][1,4]oxazin-3-one (35 mg, 30%%) as colorless oil. MS (ESI): m/z=374.4 [M+H]⁺.

### Step a) tert-Butyl 3-(4-isopropoxyphenyl)azetidine-1-carboxylate

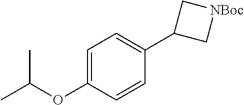 [SMILES: CC(C)Oc1ccc(C2CN(C(=O)OC(C)(C)C)C2)cc1]

A mixture of tert-butyl 3-(4-hydroxyphenyl)azetidine-1-carboxylate (Step a, Example 3) (300 mg, 1.2 mmol), 2-bromopropane (148 mg, 1.2 mmol) and cesium carbonate (588 mg, 1.8 mmol) in ACN (10 mL) was stirred at 60° C. for 16 h. The mixture was poured into brine (20 mL) and extracted with ethyl acetate (2×20 mL). The combined organic layers were concentrated under vacuum to give tert-butyl 3-(4-isopropoxyphenyl)azetidine-1-carboxylate (310 mg, 87%) as colorless oil. MS (ESI): m/z=236.4 [M+H]⁺.

### Step b) 3-(4-Isopropoxyphenyl)azetidine hydrochloride

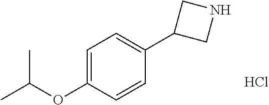

A solution of tert-butyl 3-(4-isopropoxyphenyl)azetidine-1-carboxylate (310 mg, 1.06 mmol) in HCl/ethyl acetate (6.7 mL, 27 mmol) was stirred at 20° C. for 4 h. The solution was concentrated under vacuum to give 3-(4-isopropoxyphenyl)azetidine hydrochloride (227 mg, 87%) as yellow solid. MS (ESI): m/z=192.1 [M+H]⁺.

### Example 5

### (4aR,8aS)-6-[3-(4-Tetrahydropyran-4-ylphenyl)azetidine-1-carbonyl]-4,4a,5,7,8,8a-hexahydropyrido[4,3-b][1,4]oxazin-3-one

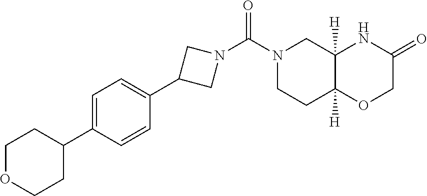 [SMILES: O=C1CO[C@H]2CCN(C(=O)N3CC(c4ccc(C5CCOCC5)cc4)C3)C[C@H]2N1]

To a stirred solution at 60° C. of 3-(4-tetrahydropyran-4-ylphenyl)azetidine hydrochloride (70 mg, 0.28 mmol) and (4-nitrophenyl) (4aR,8aS)-3-oxo-4,4a,5,7,8,8a-hexahydropyrido[4,3-b][1,4]oxazine-6-carboxylate (89 mg, 0.28 mmol) in ACN (5 mL) was added N,N-diisopropylethylamine (0.1 mL, 0.550 mmol, 2 eq). The solution was stirred at 60° C. for 16 h, then it was concentrated under vacuum to give a residue, which was purified by Prep-HPLC (TFA condition). A second purification by Prep-TLC (EtOAc) afforded (4aR,8aS)-6-[3-(4-tetrahydropyran-4-ylphenyl)azetidine-1-carbonyl]-4,4a,5,7,8,8a-hexahydropyrido[4,3-b][1,4]oxazin-3-one (22 mg, 20%) as white solid. MS (ESI): m/z=400.3 [M+H]⁺.

### Step a) tert-Butyl 3-(4-tetrahydropyran-4-ylphenyl)azetidine-1-carboxylate

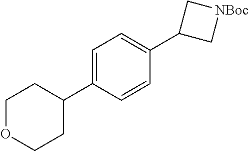 [SMILES: CC(C)(C)OC(=O)N1CC(c2ccc(C3CCOCC3)cc2)C1]

This material was prepared in analogy to example 6, step a) starting from 4-bromo-tetrahydropyran (317 mg, 1.92 mmol) and tert-butyl 3-(4-bromophenyl)azetidine-1-carboxylate (600 mg, 1.92 mmol). 502 mg (82%), light yellow solid. MS (ESI): m/z=262.0 [M-56+H]⁺.

### Step b) 3-(4-Tetrahydropyran-4-ylphenyl)azetidine hydrochloride

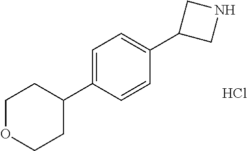 [SMILES: c1cc(C2CNC2)ccc1C1CCOCC1 ; Cl]

A solution of tert-butyl 3-(4-tetrahydropyran-4-ylphenyl)azetidine-1-carboxylate (500 mg, 1.58 mmol) in HCl/ethyl acetate (10 mL, 40 mmol) was stirred at 20° C. for 4 h. The solution was concentrated under vacuum to give 3-(4-tetrahydropyran-4-ylphenyl)azetidine hydrochloride (399 mg, 75%) as yellow oil. MS (ESI): m/z=218.5 [M+H]⁺.

### Example 6

### (4aR,8aS)-6-[3-[4-(3,3-Difluorocyclobutyl)phenyl]azetidine-1-carbonyl]-4,4a,5,7,8,8a-hexahydropyrido[4,3-b][1,4]oxazin-3-one

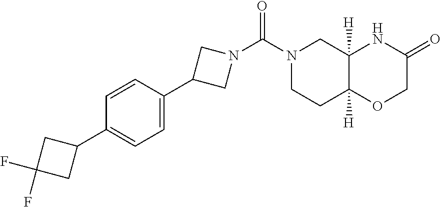 [SMILES: O=C1CO[C@H]2CCN(C(=O)N3CC(c4ccc(C5CC(F)(F)C5)cc4)C3)C[C@H]2N1]

A solution of 3-[4-(3,3-difluorocyclobutyl)phenyl]azetidine; trifluoroacetic acid salt (100.0 mg, 0.300 mmol), (4-nitrophenyl) (4aR,8aS)-3-oxo-4,4a,5,7,8,8a-hexahydropyrido[4,3-b][1,4]oxazine-6-carboxylate (142.9 mg, 0.440 mmol), and DIEA (229.9 mg, 1.78 mmol) in ACN (2 mL) was stirred at 80° C. for 12 h. The mixture was evaporated and the residue was purified by prep-HPLC (0.225% v/v FA in water and MeCN) to give the desired product (55.2 mg, 43.3%) as light brown solid. MS (ESI): m/z=406.4 [M+H]⁺.

### Step a) tert-Butyl 3-[4-(3-oxocyclobutyl)phenyl]azetidine-1-carboxylate

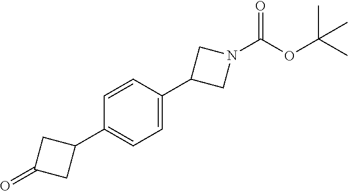 [SMILES: CC(C)(C)OC(=O)N1CC(c2ccc(C3CC(=O)C3)cc2)C1]

To a 40 mL vial equipped with a stirring bar was added 3-(4-bromophenyl)cyclobutanone (333.7 mg, 1.48 mmol, CAS RN 254892-91-6), tert-butyl 3-bromoazetidine-1-carboxylate (350.0 mg, 1.48 mmol, CAS RN 1064194-10-0), Ir[dF(CF₃)ppy]₂(dtbbpy)PF₆ (16.6 mg, 0.010 mmol, CAS RN 870987-63-6), NiC₂-glyme (1.63 mg, 0.010 mmol, CAS RN 29046-78-4), 4-tert-butyl-2-(4-tert-butyl-2-pyridyl)pyridine (2.39 mg, 0.010 mmol, CAS RN 69641-93-6), bis(trimethylsilyl)silyl-trimethyl-silane (368.6 mg, 1.48 mmol, CAS RN 1873-77-4) and Na₂CO₃ (314.2 mg, 2.96 mmol, CAS RN 497-19-8) in DME (19.4 mL). The vial was sealed and placed under nitrogen. The reaction mixture was stirred and irradiated with a 34 W blue LED lamp (7 cm distance), with cooling fan to keep the reaction temperature at 25° C., for 14 h. The reaction mixture was filtered, and the filtrate was purified by silica gel column chromatography (PE:EtOAc=20:1) to give the desired product (250 mg, 56%) as dark brown oil which was used without further purification in the next step.

### Step b) tert-Butyl 3-[4-(3,3-difluorocyclobutyl)phenyl]azetidine-1-carboxylate

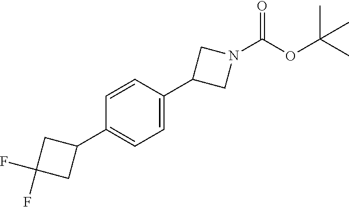 [SMILES: CC(C)(C)OC(=O)N1CC(c2ccc(C3CC(F)(F)C3)cc2)C1]

To a solution of tert-butyl 3-[4-(3-oxocyclobutyl)phenyl]azetidine-1-carboxylate (250.0 mg, 0.830 mmol) in DCM (5 mL) was added DAST (668.6 mg, 4.15 mmol) at −10° C., then the mixture was stirred at 40° C. for 12 h. The reaction mixture was poured into saturated aq. NaHCO₃ solution and extracted with EtOAc. The organic layer was evaporated under reduced pressure, and the residue was purified by reversed flash chromatography (0.1% v/v FA in water and ACN) to give the desired product (130 mg, 48.5% yield) as dark brown oil. MS (ESI): m/z=268.6 [M-C₄H₈+H]⁺.

### Step c) 3-[4-(3,3-Difluorocyclobutyl)phenyl]azetidine, Trifluoroacetic Acid Salt

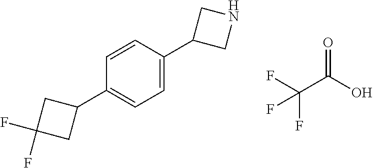 [SMILES: FC1(F)CC(c2ccc(C3CNC3)cc2)C1 ; O=C(O)C(F)(F)F]

To a solution of tert-butyl 3-[4-(3,3-difluorocyclobutyl)phenyl]azetidine-1-carboxylate (100.0 mg, 0.310 mmol) in DCM (3 mL) was added TFA (0.65 mL, 8.44 mmol) and the solution stirred at 20° C. for 12 h. The mixture was evaporated to give the desired crude product as light brown oil (100 mg, 95.9%). MS (ESI): m/z=224.6 [M+H]⁺.

### Example 7

### (4aR,8aS)-6-[3-[5-(2,4-Dichlorophenyl)-1,2,4-thiadiazol-3-yl]azetidine-1-carbonyl]-4,4a,5,7,8,8a-hexahydropyrido[4,3-b][1,4]oxazin-3-one

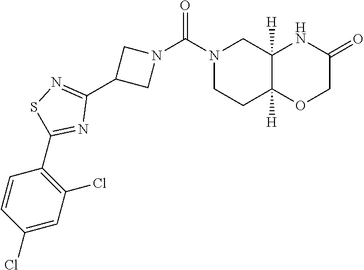 [SMILES: O=C1CO[C@H]2CCN(C(=O)N3CC(c4nsc(-c5ccc(Cl)cc5Cl)n4)C3)C[C@H]2N1]

To a solution of 3-(azetidin-3-yl)-5-(2,4-dichlorophenyl)-1,2,4-thiadiazole; trifluoroacetic acid salt (120.0 mg, 0.300 mmol) in ACN (5 mL) was added DIPEA (232.1 mg, 1.8 mmol) and (4-nitrophenyl) (4aR,8aS)-3-oxo-4,4a,5,7,8,8a-hexahydropyrido[4,3-b][1,4]oxazine-6-carboxylate (115.6 mg, 0.360 mmol). The mixture was stirred at 80° C. for 12 h. Then the mixture was concentrated, and the residue was purified by prep-HPLC (0.225% v/v FA in water and ACN) to give the desired product as off-white solid (52.7 mg, 37.5%). MS (ESI): m/z=468.2 [M+H]⁺.

### Step a) tert-Butyl 3-(N-hydroxycarbamimidoyl)azetidine-1-carboxylate

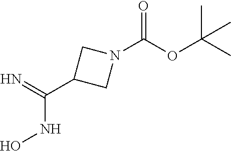

To a solution of hydroxylamine hydrochloride (1.53 g, 22.0 mmol) and 1-boc-3-cyanoazetidine (2.0 g, 11.0 mmol, CAS RN 142253-54-1) in MeOH (20 mL) and water (20 mL) was added sodium carbonate (2.33 g, 22.0 mmol), the mixture was stirred at 50° C. for 12 h. The mixture was filtered, the filtrate concentrated under vacuum and the mixture extracted twice with EtOAc (50 mL each). The combined organic players were dried over Na₂SO₄, filtered and concentrated to give the desired product (1.8 g, 76.2%) as light yellow solid. MS (ESI): m/z=160.2 [M-56+H]⁺.

### Step b) tert-Butyl 3-carbamimidoylazetidine-1-carboxylate, Acetic Acid Salt

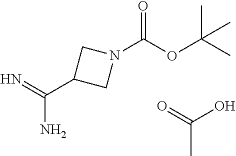

To a solution of tert-butyl 3-(N-hydroxycarbamimidoyl)azetidine-1-carboxylate (1000.0 mg, 4.65 mmol) and acetic anhydride (711.4 mg, 6.97 mmol) in AcOH (30 mL) was added wet Pd/C (wt. 10%, 300.0 mg) and the mixture was stirred at 20° C. under H₂ atmosphere for 12 h. The mixture was diluted with MeOH (50 mL) and then filtered. The filtrate was evaporated and the residue dried to give the desired product as light yellow oil (1100 mg, 91.3%). MS (ESI): m/z=200.2 [M+H]⁺.

### Step c) tert-Butyl 3-(5-amino-1,2,4-thiadiazol-3-yl)azetidine-1-carboxylate

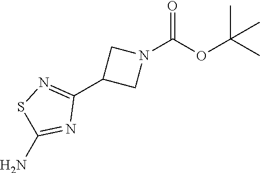 [SMILES: CC(C)(C)OC(=O)N1CC(c2nsc(N)n2)C1]

To a solution of tert-butyl 3-carbamimidoylazetidine-1-carboxylate; acetic acid salt (1000.0 mg, 3.86 mmol) in water (10 mL) was added sodium hypochlorite (6.1 mL, 4.24 mmol, 0.7M in water) at 0° C. The mixture was stirred at 20° C. for 1 h, diluted with water and extracted three times with EtOAc (10 mL each). The combined organic layers were dried over Na₂SO₄, concentrated, filtered and evaporated. The residue was dissolved in MeOH (10 mL) and potassium thiocyanate (412.3 mg, 4.24 mmol) was added at 0° C. Then the solution was stirred at 20° C. for 11 h and the mixture was concentrated. The residue was dissolved in EtOAc (30 mL), filtered and the filtrate was concentrated. The residue was purified by silica gel column (PE:EtOAc=1:1) to give the desired product as yellow oil (450 mg, 45.5%). MS (ESI): m/z=201.1 [M-56+H]⁺.

### Step d) tert-Butyl 3-(5-bromo-1,2,4-thiadiazol-3-yl)azetidine-1-carboxylate

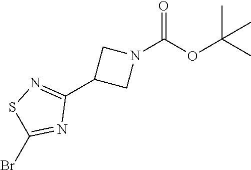 [SMILES: CC(C)(C)OC(=O)N1CC(c2nsc(Br)n2)C1]

To a solution of tert-butyl 3-(5-amino-1,2,4-thiadiazol-3-yl)azetidine-1-carboxylate (600.0 mg, 2.34 mmol) and copper(II) bromide (784.2 mg, 3.51 mmol) in MeCN (18 mL) was added tert-butyl nitrite (362.1 mg, 3.51 mmol) at 0° C. The mixture was stirred at 20° C. for 12 h. The mixture was poured into water (20 mL) and extracted three times with EtOAc (20 mL each). The combined organic layers were concentrated and purified by silica gel column chromatography (PE:EtOAc=1:1) to give the desired product as yellow gum (350 mg, 46.4%). MS (ESI): m/z=264.2 [M-C₄H₈+H]⁺.

### Step e) tert-Butyl 3-[5-(2,4-dichlorophenyl)-1,2,4-thiadiazol-3-yl]azetidine-1-carboxylate

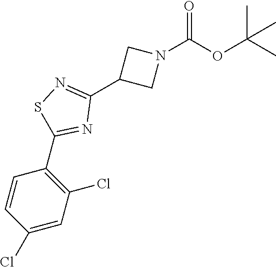 [SMILES: CC(C)(C)OC(=O)N1CC(c2nsc(-c3ccc(Cl)cc3Cl)n2)C1]

To a solution of tert-butyl 3-(5-bromo-1,2,4-thiadiazol-3-yl)azetidine-1-carboxylate (250.0 mg, 0.780 mmol), 2,4-dichlorophenylboronic acid (148.98 mg, 0.780 mmol) and Na₂CO₃ (165.5 mg, 1.56 mmol) in 1,4-dioxane (8 mL) and water (2 mL) was added Pd(dppf)Cl₂ (57.1 mg, 0.080 mmol) and the mixture was stirred at 100° C. under N₂ atmosphere for 12 h. Then the mixture was filtered, the filtrate concentrated and the residue was purified by silica gel column (PE:EtOAc=20:1) to give the desired product as light yellow solid (250 mg, 82.9%). MS (ESI): m/z=329.9 [M-C₄H₈+H]⁺.

### Step f) 3-(Azetidin-3-yl)-5-(2,4-dichlorophenyl)-1,2,4-thiadiazole (Trifluoroacetic Acid Salt)

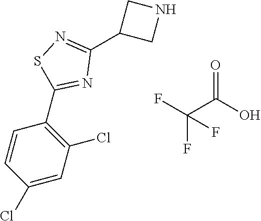 [SMILES: Clc1ccc(-c2nc(C3CNC3)ns2)c(Cl)c1 ; O=C(O)C(F)(F)F]

To a solution of tert-butyl 3-[5-(2,4-dichlorophenyl)-1,2,4-thiadiazol-3-yl]azetidine-1-carboxylate (150.0 mg, 0.390 mmol) in DCM (2.5 mL) was added TFA (0.5 mL, 0.390 mmol). The mixture was stirred at 20° C. for 1 h and then concentrated to give the crude product as light brown oil (150 mg, 96.5%). MS (ESI): m/z=286.2 [M+H]⁺.

### Example 8

### (4aR,8aS)-6-[3-[1-(2,4-Dichlorophenyl)imidazol-4-yl]azetidine-1-carbonyl]-4,4a,5,7,8,8a-hexahydropyrido[4,3-b][1,4]oxazin-3-one

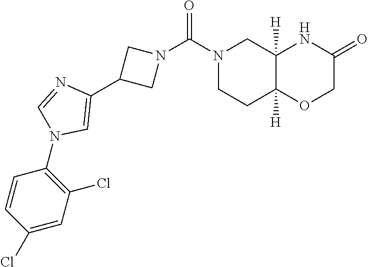 [SMILES: O=C1CO[C@H]2CCN(C(=O)N3CC(c4cn(-c5ccc(Cl)cc5Cl)cn4)C3)C[C@H]2N1]

A mixture of (4-nitrophenyl) (4aR,8aS)-3-oxo-4,4a,5,7,8,8a-hexahydropyrido[4,3-b][1,4]oxazine-6-carboxylate (151.3 mg, 0.470 mmol), DIEA (202.8 mg, 1.57 mmol) and 4-(azetidin-3-yl)-1-(2,4-dichlorophenyl)imidazole; 2,2,2-trifluoroacetic acid salt (60.0 mg, 0.160 mmol) in ACN (1.9 mL) was stirred at 80° C. for 12 h. The mixture was concentrated and the residue purified by prep-HPLC (0.225% v/v FA in water and ACN) to give the desired compound as yellow solid (13 mg, 17.6%). MS (ESI): m/z=450.2 [M+H]⁺.

### Step a) tert-Butyl 3-methanimidoylazetidine-1-carboxylate

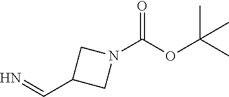

The solution of tert-butyl 3-formylazetidine-1-carboxylate (2000.0 mg, 10.8 mmol) in NH₃/MeOH (7M, 21.5 mL) was stirred at 60° C. for 2 h. The reaction mixture was evaporated under reduced pressure to give the title compound as yellow oil (1989 mg, 99.9% yield) which was used without further purification.

### Step b) tert-Butyl 3-(1H-imidazol-4-yl)azetidine-1-carboxylate

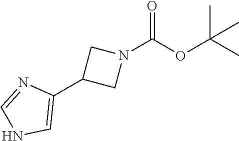 [SMILES: CC(C)(C)OC(=O)N1CC(c2c[nH]cn2)C1]

A solution of tert-butyl 3-methanimidoylazetidine-1-carboxylate (1989.0 mg, 10.8 mmol), tosylmethyl isocyanide (2318.5 mg, 11.9 mmol) and K₂CO₃ (2977.7 mg, 21.6 mmol) in EtOH (50 mL) was stirred at 78° C. for 12 h. The reaction mixture was evaporated, the residue poured into water, extracted with EtOAc and concentrated to give the crude product, which was purified by reversed-phase column chromatography and prep-HPLC (0.5% v/v ammonia in water and ACN) to give the title compound as light yellow gum (100 mg, 4.2%). MS (ESI): m/z=168.2 [M-56+H]⁺.

### Step c) tert-Butyl 3-[1-(2,4-dichlorophenyl)imidazol-4-yl]azetidine-1-carboxylate

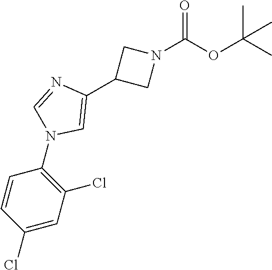 [SMILES: CC(C)(C)OC(=O)N1CC(c2cn(-c3ccc(Cl)cc3Cl)cn2)C1]

A solution of tert-butyl 3-(1H-imidazol-4-yl)azetidine-1-carboxylate (100.0 mg, 0.450 mmol), 2,4-dichlorophenylboronic acid (102.6 mg, 0.540 mmol), Cu(OAc)₂ (162.1 mg, 0.90 mmol) and TEA (0.2 mL, 1.79 mmol) in DCM (10 mL) was purged with oxygen for 3 times and the mixture was stirred at 25° C. for 12 h. The reaction mixture was filtered through celite, the filter cake washed with DCM, the filtrate evaporated and the residue was purified with prep-HPLC (0.225% v/v FA in water and ACN) to give the title compound as yellow gum (80 mg, 48.5%). MS (ESI): m/z=312.0 [M-56+H]⁺.

### Step d) 4-(azetidin-3-yl)-1-(2,4-dichlorophenyl)imidazole 2,2,2-trifluoroacetic Acid Salt

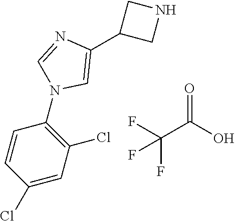

To a solution of tert-butyl 3-[1-(2,4-dichlorophenyl)imidazol-4-yl]azetidine-1-carboxylate (80.0 mg, 0.220 mmol) in DCM (3 mL) was added TFA (0.5 mL) and the mixture was stirred at 20° C. for 12 h. The reaction mixture was evaporated to give the desired compound as yellow oil (70 mg, 84.3%). MS (ESI): m/z=268.1 [M+H]⁺.

### Example 9

### (4aR,8aS)-6-[3-[3-[4-Chloro-2-(trifluoromethyl)phenyl]-1,2,4-oxadiazol-5-yl]azetidine-1-carbonyl]-4,4a,5,7,8,8a-hexahydropyrido[4,3-b][1,4]oxazin-3-one

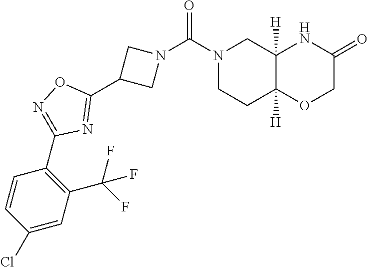 [SMILES: O=C1CO[C@H]2CCN(C(=O)N3CC(c4nc(-c5ccc(Cl)cc5C(F)(F)F)no4)C3)C[C@H]2N1]

To a solution of 5-(azetidin-3-yl)-3-[4-chloro-2-(trifluoromethyl)phenyl]-1,2,4-oxadiazole; trifluoroacetic acid salt (50.0 mg, 0.120 mmol) in ACN (2 mL) were added DIPEA (92.65 mg, 0.720 mmol) and (4-nitrophenyl) (4aR,8aS)-3-oxo-4,4a,5,7,8,8a-hexahydropyrido[4,3-b][1,4]oxazine-6-carboxylate (46.15 mg, 0.140 mmol). The mixture was stirred at 80° C. for 12 h, evaporated, and the residue was purified by prep-HPLC (0.225% v/v FA in water ACN) to give the desired product as light brown solid (20.2 mg, 34.5%). MS (ESI): m/z=486.2 [M+H]⁺.

### Step a) 2-Chloro-N-hydroxy-4-(trifluoromethyl)benzamidine

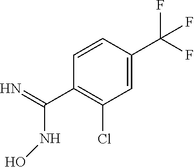 [SMILES: N=C(NO)c1ccc(C(F)(F)F)cc1Cl]

To a solution of hydroxylamine hydrochloride (0.44 g, 6.32 mmol, CAS RN 5470-11-1) in EtOH (9 mL) was added sodium carbonate (0.34 g, 3.16 mmol, CAS RN 497-19-8) in water (1.8 mL), and the mixture was stirred at 20° C. for 25 min. To the mixture was added 2-chloro-4-(trifluoromethyl)benzonitrile (1.0 g, 4.86 mmol, CAS RN 1813-33-8) and the mixture was stirred at 90° C. for 12 h. The mixture was diluted with water and concentrated under vacuum. The residue was purified by reversed flash chromatography (0.1% v/v FA in water and MeCN) to give the desired product as light yellow oil (1000 mg, 86.2%). MS (ESI): m/z=224.6 [M-14+H]⁺.

### Step b) tert-Butyl 3-[3-[4-chloro-2-(trifluoromethyl)phenyl]-1,2,4-oxadiazol-5-yl]azetidine-1-carboxylate

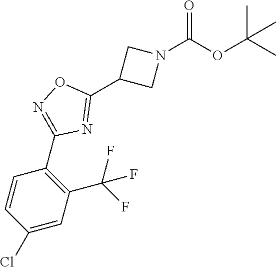 [SMILES: CC(C)(C)OC(=O)N1CC(c2nc(-c3ccc(Cl)cc3C(F)(F)F)no2)C1]

To a solution of 2,4-dichloro-N-hydroxy-benzamidine (400.0 mg, 1.95 mmol), 1-boc-azetidine-3-carboxylic acid (471.1 mg, 2.34 mmol) and HATU (890.1 mg, 2.34 mmol) in DCM (8 mL) was added DIPEA (756.4 mg, 5.85 mmol). The mixture was stirred at 20° C. for 16 h. The mixture was evaporated and the crude product purified by silica gel column chromatography (PE:EtOAc=3:1) to give the desired product as light brown oil (50 mg, 2.9%). MS (ESI): m/z=348.1 [M-56+H]⁺.

### Step c) 5-(Azetidin-3-yl)-3-[4-chloro-2-(trifluoromethyl)phenyl]-1,2,4-oxadiazole; Trifluoroacetic Acid Salt

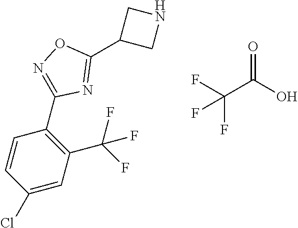 [SMILES: FC(F)(F)c1cc(Cl)ccc1-c1noc(C2CNC2)n1 ; O=C(O)C(F)(F)F]

To a solution of tert-butyl 3-[3-[4-chloro-2-(trifluoromethyl)phenyl]-1,2,4-oxadiazol-5-yl]azetidine-1-carboxylate (50.0 mg, 0.120 mmol) in DCM (1 mL) was added TFA (0.11 mL, 1.49 mmol). After stirring at 20° C. for 12 h the mixture was evaporated to give the crude product as light brown oil (50 mg, 96.7%). MS (ESI): m/z=304.0 [M+H]⁺.

### Example 10

### (4aR,8aS)-6-(3-(2,2-Dimethylchroman-6-yl)azetidine-1-carbonyl)hexahydro-2H-pyrido[4,3-b][1,4]oxazin-3(4H)-one

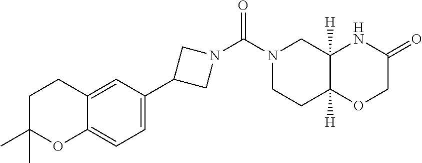 [SMILES: CC1(C)CCc2cc(C3CN(C(=O)N4CC[C@@H]5OCC(=O)N[C@@H]5C4)C3)ccc2O1]

To a mixture of 4-nitrophenyl (4aR,8aS)-3-oxohexahydro-2H-pyrido[4,3-b][1,4]oxazine-6(5H)-carboxylate (50.7 mg, 158 μmol) and 3-(2,2-dimethylchroman-6-yl)azetidine 4-methylbenzenesulfonate (123 mg, 316 μmol) in ACN (0.7 mL) was added DIPEA (163 mg, 221 μL, 1.26 mmol) and the mixture was stirred at RT for 2.75 h. Then the clear, yellow solution was stirred at 50° C. for 20 min. The product was purified by prep-HPLC (Gemini NX column) using a gradient of ACN:water (containing 0.1% TEA) (20:80 to 98:2) to yield the desired compound as a colorless solid (0.035 g; 27.7%). MS (ESI): m/z=400.2 [M+H]⁺.

### Step a) tert-Butyl 3-(2,2-dimethylchroman-6-yl)azetidine-1-carboxylate

To an 20 mL vial equipped with a stirring bar was added (Ir[dF(CF₃)ppy]2(dtbpy))PF₆ (18.6 mg, 16.6 μmol, CAS RN 870987-63-6), 6-bromo-2,2-dimethylchromane (400 mg, 1.66 mmol, CAS RN 174894-80-5), tert-butyl 3-bromoazetidine-1-carboxylate (588 mg, 407 μL, 2.49 mmol, CAS RN 1064194-10-0), tris(trimethylsilyl)silane (412 mg, 512 μL, 1.66 mmol) and anhydrous sodium carbonate (352 mg, 3.32 mmol). The vial was sealed and placed under argon before DME (10 ml) was added. To a separate vial was added nickel(II) chloride ethylene glycol dimethyl ether complex (3.64 mg, 16.6 μmol) and 4,4′-di-tert-butyl-2,2′-bipyridine (4.45 mg, 16.6 μmol). The precatalyst vial was sealed, purged with argon then to it was added DME (4 ml). The precatalyst vial was sonicated for 5 min, after which 1 mL (0.5 mol % catalyst, 0.005 eq) of it was syringed into the reaction vessel. The reaction suspension was degassed with argon. The reaction was stirred and irradiated with a 420 nm lamp for 1 h. The filtrate was treated with silica gel and evaporated. The compound was purified by silica gel chromatography on a 80 g column using an MPLC (ISCO) system eluting with an isocratic mixture of n-heptane:TBME (60:40) to get the desired compound as a colorless solid (0.459 g; 87.2%). MS (ESI): m/z=262.2 [M-56+H]⁺.

### Step b) 3-(2,2-Dimethylchroman-6-yl)azetidine 4-methylbenzenesulfonate

To a solution of tert-butyl 3-(2,2-dimethylchroman-6-yl)azetidine-1-carboxylate (100 mg, 315 μmol) in EtOAc (1 mL) was added p-toluenesulfonic acid monohydrate (59.9 mg, 315 μmol) and the mixture was stirred at RT overnight. The mixture was completely evaporated and the residue was used without further purification in the next step. MS (ESI): m/z=218.2 [M+H]⁺.

### Example 11

### (4aR,8aS)-6-(3-(4-Isobutoxyphenyl)azetidine-1-carbonyl)hexahydro-2H-pyrido[4,3-b][1,4]oxazin-3(4H)-one

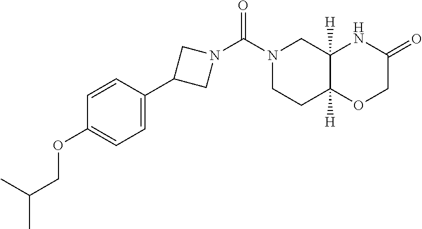 [SMILES: CC(C)COc1ccc(C2CN(C(=O)N3CC[C@@H]4OCC(=O)N[C@@H]4C3)C2)cc1]

To a suspension of (4aR,8aS)-6-(3-(4-hydroxyphenyl)azetidine-1-carbonyl)hexahydro-2H-pyrido[4,3-b][1,4]oxazin-3(4H)-one (0.056 g, 169 μmol) and potassium carbonate (28 mg, 203 μmol) in DMF (0.7 mL) was added 1-iodo-2-methylpropane (23.3 μL, 203 μmol) and the mixture was stirred at RT overnight. Stirring was continued at 50° C. for 3 h. Another batch of 1-iodo-2-methylpropane (23.3 μL, 203 μmol) and potassium carbonate (28 mg, 203 μmol) were added and stirring was continued at 50° C. overnight. Another batch of 1-iodo-2-methylpropane (38.9 μL, 338 μmol) was added and stirring was continued at 50° C. for another 2 h. The mixture was filtered, the filter cake was washed with a few drops of DMF. The product was purified by prep-HPLC (Gemini NX column) using a gradient of ACN:water (containing 0.1% HCOOH) (20:80 to 100:0) to provide the desired compound as a colorless solid (0.008 g; 12.2%). MS (ESI): m/z=388.2 [M+H]⁺.

### Step a) tert-Butyl 3-(4-hydroxyphenyl)azetidine-1-carboxylate

To a 40 mL vial equipped with a stirring bar was added 4-bromophenol (3663.8 mg, 21.2 mmol), tert-butyl 3-bromoazetidine-1-carboxylate (5000.0 mg, 21.2 mmol), Ir[dF(CF₃)ppy]2(dtbbpy)PF₆ (237.39 mg, 0.210 mmol, CAS RN 870987-63-6), NiCl₂-glyme (23.3 mg, 0.110 mmol), 4-tert-butyl-2-(4-tert-butyl-2-pyridyl)pyridine (34.1 mg, 0.130 mmol), bis(trimethylsilyl)silyl-trimethyl-silane (5265.8 mg, 21.2 mmol) and Na₂CO₃ (4489 mg, 42.4 mmol) in DME (100 mL). The vial was sealed and placed under nitrogen was added. The reaction was stirred and irradiated with a 34 W blue LED lamp (7 cm distance) with a cooling fan to keep the reaction temperature at 25° C. for 14 h. The reaction was filtered and the filtrate was purified by prep-HPLC (FA) and lyophilized to give the desired product as a light yellow solid (1800 mg, 7.22 mmol, 34.1%). MS (ESI): m/z=194.0 [M-56+H]⁺.

### Step b) 4-(Azetidin-3-yl)phenol; 2,2,2-trifluoroacetic Acid

To a solution of tert-butyl 3-(4-hydroxyphenyl)azetidine-1-carboxylate (500.0 mg, 2.0 mmol) in DCM (15 mL) was added TFA (5.0 mL, 2.0 mmol), the mixture was stirred at 20° C. for 12 h. The reaction mixture was evaporated to give the desired product as a yellow oil (520 mg, 98.5%). MS (ESI): m/z=149.9 [M+H]⁺.

### Step c) (4aR,8aS)-6-(3-(4-Hydroxyphenyl)azetidine-1-carbonyl)hexahydro-2H-pyrido[4,3-b][1,4]oxazin-3(4H)-one

A mixture of (4-nitrophenyl) (4aR,8aS)-3-oxo-4,4a,5,7,8,8a-hexahydropyrido[4,3-b][1,4]oxazine-6-carboxylate (610.3 mg, 1.9 mmol), DIPEA (2453.4 mg, 19 mmol) and 4-(azetidin-3-yl)phenol; 2,2,2-trifluoroacetic acid (500.0 mg, 1.9 mmol) in ACN (10 mL) was stirred at 80° C. for 12 h. The mixture was evaporated under reduced pressure. To the residue ACN (20 mL) was added upon which a white solid formed. The solution was filtered and the filter cake was collected and dried to give the desired product as a white solid (350 mg, 55.6%). MS (ESI): m/z=332.1 [M+H]⁺.

### Example 12

### (4aR,8aS)-6-[3-[4-(2,2,2-Trifluoro-1,1-dimethyl-ethoxy)phenyl]azetidine-1-carbonyl]-4,4a,5,7,8,8a-hexahydropyrido[4,3-b][1,4]oxazin-3-one

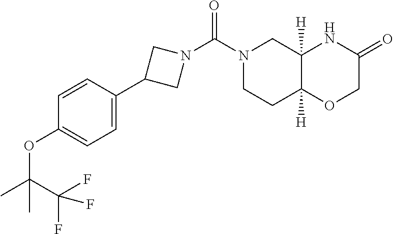 [SMILES: CC(C)(Oc1ccc(C2CN(C(=O)N3CC[C@@H]4OCC(=O)N[C@@H]4C3)C2)cc1)C(F)(F)F]

To a solution of 3-[4-(2,2,2-trifluoro-1,1-dimethyl-ethoxy)phenyl]azetidine; trifluoroacetic acid salt (50.0 mg, 0.130 mmol) and DIPEA (103.67 mg, 0.800 mmol) in ACN (1.5 mL) was added (4-nitrophenyl) (4aR,8aS)-3-oxo-4,4a,5,7,8,8a-hexahydropyrido[4,3-b][1,4]oxazine-6-carboxylate (64.55 mg, 0.200 mmol) and the mixture was stirred at 80° C. for 12 h. The mixture was evaporated and the residue was purified by prep-HPLC (0.225% v/v FA in water and ACN) to give the desired product as white solid (20.8 mg, 33.8%). MS (ESI): m/z=442.1 [M+H]⁺.

### Step a) 1-Nitro-4-(2,2,2-trifluoro-1,1-dimethyl-ethoxy)benzene

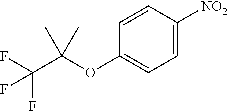 [SMILES: CC(C)(Oc1ccc([N+](=O)[O-])cc1)C(F)(F)F]

To a solution of 1-fluoro-4-nitro-benzene (2000.0 mg, 14.17 mmol, CAS RN 350-46-9) and 2-trifluoromethyl-2-propanol (1997.15 mg, 15.59 mmol, CAS RN 507-52-8) in DMF (50 mL) was added NaH (1133.95 mg, 28.35 mmol) and the mixture was stirred at 20° C. for 12 h. The mixture was poured into ice water and extracted three times with EtOAc (100 mL each). The combined organic layers were washed with brine, dried over Na₂SO₄ and filtered.

The filtrate was concentrated under vacuum and the residue purified by column chromatography (PE:EtOAc=1:1˜50:1) to give the desired product as light yellow gum (1.2 g, 34%).

### Step b) 4-(2,2,2-Trifluoro-1,1-Dimethyl-ethoxy)aniline

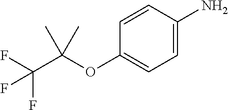

To a mixture of 1-nitro-4-(2,2,2-trifluoro-1,1-dimethyl-ethoxy)benzene (1.0 g, 4.01 mmol) and acetic acid (2.41 g, 40.13 mmol) in THF (40 mL) was added zinc dust (1.31 g, 20.07 mmol) portion-wise. The mixture was stirred at 20° C. for 16 h. The mixture was filtered and the filtrate concentrated to give the desired product as light yellow solid (800 mg, 91%). MS (ESI): m/z=220.0 [M+H]⁺.

### Step a) I-Bromo-4-(2,2,2-trifluoro-1,1-dimethyl-ethoxy)benzene

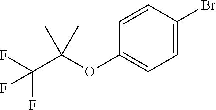

A solution of 4-(2,2,2-trifluoro-1,1-dimethyl-ethoxy)aniline (300.0 mg, 1.37 mmol) in ACN (7 mL) was cooled down to 0° C. and CuBr₂ (397.39 mg, 1.78 mmol) and tert-butyl nitrite (0.23 mL, 1.78 mmol) were added. The reaction was stirred at 0° C. for 10 min, then at 70° C. for 16 h. The mixture was diluted with EtOAc (200 mL) and washed with ammonia (4M, 100 mL) followed by brine. The organic phase was dried over Na₂SO₄ and concentrated to give the desired product as light brown oil (180 mg, 46.5%).

### Step c) tert-Butyl 3-[4-(2,2,2-trifluoro-1,1-dimethyl-ethoxy)phenyl]azetidine-1-carboxylate

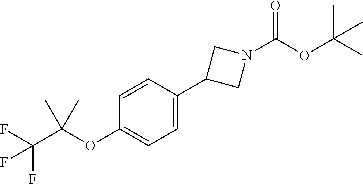 [SMILES: CC(C)(C)OC(=O)N1CC(c2ccc(OC(C)(C)C(F)(F)F)cc2)C1]

To a 40 mL vial equipped with a stirring bar was added tert-butyl 3-bromoazetidine-1-carboxylate (83.4 mg, 0.350 mmol), 1-bromo-4-(2,2,2-trifluoro-1,1-dimethyl-ethoxy)benzene (100.0 mg, 0.350 mmol), Ir[dF(CF₃)ppy]2(dtbbpy)PF₆ (3.96 mg), NiCl₂ glyme (0.39 mg), 4-tert-butyl-2-(4-tert-butyl-2-pyridyl)pyridine (0.57 mg), bis(trimethylsilyl)silyl-trimethyl-silane (87.84 mg, 0.350 mmol) and Na₂CO₃ (74.88 mg, 0.710 mmol) in DME (4.5 mL). The vial was sealed and placed under nitrogen. The reaction mixture was stirred and irradiated with a 34 W blue LED lamp (7 cm distance), with cooling fan to keep the reaction temperature at 25° C. for 14 hr. The reaction mixture was filtered, the filtrate evaporated and the crude product purified by reversed flash chromatography (0.1% v/v FA in water and ACN) to give the desired product as light brown oil (50 mg, 39.4%). MS (ESI): m/z=304.1 [M-56+H]⁺.

### Step d) 3-[4-(2,2,2-Trifluoro-,1-Dimethyl-ethoxy)phenyl]azetidine (Trifluoroacetic Acid Salt)

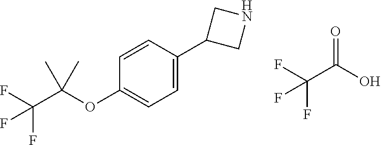

To a solution of tert-butyl 3-[4-(2,2,2-trifluoro-1,1-dimethyl-ethoxy)phenyl]azetidine-1-carboxylate (50.0 mg, 0.140 mmol) in DCM (2 mL) was added TFA (0.4 mL, 5.19 mmol) and the mixture stirred at 20° C. for 12 h. The mixture was evaporated to give the crude desired product as light brown oil (50 mg, 96.3% yield). MS (ESI): m/z=260.6 [M+H]⁺.

### Example 13

### (4aR,8aS)-6-(3-(4-(Cyclopentyloxy)phenyl)azetidine-1-carbonyl)hexahydro-2H-pyrido[4,3-b][1,4]oxazin-3(4H)-one

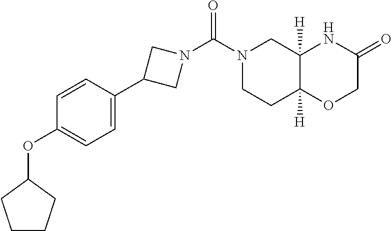 [SMILES: O=C1CO[C@H]2CCN(C(=O)N3CC(c4ccc(OC5CCCC5)cc4)C3)C[C@H]2N1]

tert-Butyl 3-(4-(cyclopentyloxy)phenyl)azetidine-1-carboxylate (150 mg, 473 μmol) was dissolved in 1,1,1,3,3,3-hexafluoropropan-2-ol (2 mL) and stirred for 40 min at 150° C. in the microwave. The solution was completely evaporated and the residue suspended in ACN (1.5 ml). 4-Nitrophenyl (4aR,8aS)-3-oxohexahydro-2H-pyrido[4,3-b][1,4]oxazine-6(5H)-carboxylate (152 mg, 473 μmol) and DIPEA (244 mg, 330 μL, 1.89 mmol) were added and the mixture was stirred at RT over night. The suspension was completely evaporated and the crude product purified by prep-HPLC (YMC-Triart column) using a gradient of ACN:water (containing 0.1% TEA) (20:80 to 40:60 to 55:45 to 0:100) to provide the desired compound as a colorless solid (0.032 g; 17%). MS (ESI): m/z=400.3 [M+H]⁺.

### Step a) tert-Butyl 3-(4-(cyclopentyloxy)phenyl)azetidine-1-carboxylate

The product was obtained in analogy to Example 10, step a, from bromo-4-(cyclopentyloxy)benzene (CAS RN 30752-30-8) as a colorless solid. MS (ESI): m/z=262.1 [M-56+H]⁺.

### Example 14

### (4aR,8aS)-6-(3-(4,4-Dimethylchroman-6-yl)azetidine-1-carbonyl)hexahydro-2H-pyrido[4,3-b][1,4]oxazin-3(4H)-one

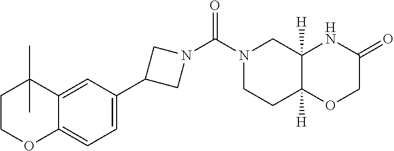

A solution of tert-butyl 3-(4,4-dimethylchroman-6-yl)azetidine-1-carboxylate (100 mg, 315 μmol) and 4-methylbenzenesulfonic acid hydrate (59.9 mg, 315 μmol) in EtOAc (1 mL) was stirred at 85° C. for 20 min. Then the reaction was quenched with DIPEA (48.9 mg, 66 μL, 378 μmol) and the suspension was completely evaporated. The residue was suspended in ACN (1 mL) and 4-nitrophenyl (4aR,8aS)-3-oxohexahydro-2H-pyrido[4,3-b][1,4]oxazine-6(5H)-carboxylate (101 mg, 315 μmol) and DIPEA (163 mg, 220 μL, 1.26 mmol) were added. The mixture was stirred at RT over night and then completely evaporated. The product was purified by prep-HPLC (Gemini NX column) using a gradient of ACN:water (containing 0.1% TEA) (15:85 to 35:65 to 50:50 to 0:100) to furnish the desired compound as a colorless solid (0.036 g; 28.6%). MS (ESI): m/z=400.3 [M+H]⁺.

### Step a) tert-Butyl 3-(4,4-dimethylchroman-6-yl)azetidine-1-carboxylate

The product was obtained in analogy to Example 10, step a, from 6-bromo-4,4-dimethylchromane (CAS RN 1027915-16-7) as a colorless oil. MS (ESI): m/z=262.1 [M-56+H]⁺.

### Example 15

### (4aR,8aS)-6-(3-(4-(Cyclopropylmethoxy)phenyl)azetidine-1-carbonyl)hexahydro-2H-pyrido[4,3-b][1,4]oxazin-3(4H)-one

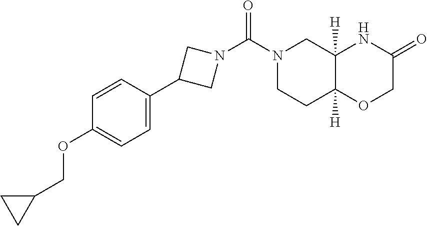 [SMILES: O=C1CO[C@H]2CCN(C(=O)N3CC(c4ccc(OCC5CC5)cc4)C3)C[C@H]2N1]

A solution of tert-butyl 3-(4-(cyclopropylmethoxy)phenyl)azetidine-1-carboxylate (150 mg, 494 μmol) and 4-methylbenzenesulfonic acid hydrate (94 mg, 494 μmol) in EtOAc (1.5 mL) was stirred at 85° C. for 20 min. Then the reaction was quenched with DIPEA (76.7 mg, 104 μL, 593 μmol) and the suspension was completely evaporated. The residue was taken up in ACN (1.5 ml) and 4-nitrophenyl (4aR,8aS)-3-oxohexahydro-2H-pyrido[4,3-b][1,4]oxazine-6(5H)-carboxylate (159 mg, 494 μmol) and DIPEA (256 mg, 345 μL, 1.98 mmol) were added. The mixture was stirred at RT over night and then completely evaporated. The product was purified by prep-HPLC (Gemini NX column) using a gradient of ACN:water (containing 0.1% TEA) (15:85 to 35:65 to 50:50) to yield the desired compound as a colorless solid (0.10 g; 52.5%). MS (ESI): m/z=386.3 [M+H]⁺.

### Step a) tert-Butyl 3-(4-(cyclopropylmethoxy)phenyl)azetidine-1-carboxylate

The product was obtained in analogy to Example 10, step a, from 1-bromo-4-(cyclopropylmethoxy)benzene (CAS RN 412004-56-9) as a colorless oil. MS (ESI): m/z=248.1 [M-56+H]⁺.

### Example 16

### (4aR,8aS)-6-[3-[3-(2,4-Dichlorophenyl)-1,2,4-oxadiazol-5-yl]azetidine-1-carbonyl]-4,4a,5,7,8,8a-hexahydropyrido[4,3-b][1,4]oxazin-3-one

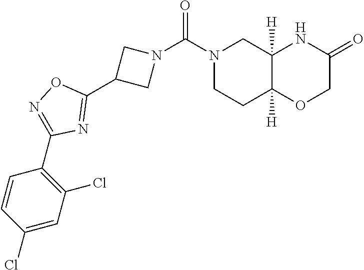 [SMILES: O=C1CO[C@H]2CCN(C(=O)N3CC(c4nc(-c5ccc(Cl)cc5Cl)no4)C3)C[C@H]2N1]

To a solution of 5-(azetidin-3-yl)-3-(2,4-dichlorophenyl)-1,2,4-oxadiazole; trifluoroacetic acid salt (125.0 mg, 0.330 mmol) in ACN (3 mL) was added DIPEA (251.9 mg, 1.95 mmol) and (4-nitrophenyl) (4aR,8aS)-3-oxo-4,4a,5,7,8,8a-hexahydropyrido[4,3-b][1,4]oxazine-6-carboxylate (125.46 mg, 0.390 mmol). The mixture was stirred at 80° C. for 12 h, evaporated and the residue was purified by prep-HPLC (0.225% v/v FA in water and ACN) to give the desired product as light yellow solid (44.6 mg, 30.3%). MS (ESI): m/z=452.2 [M+H]⁺.

### Step a) 2,4-Dichloro-N-hydroxy-benzamidine

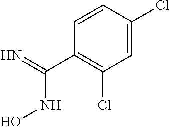 [SMILES: N=C(NO)c1ccc(Cl)cc1Cl]

A solution of hydroxylamine hydrochloride (1.05 g, 15.1 mmol, CAS RN 5470-11-1) in EtOH (21 mL) was mixed with a solution of K₂CO₃ (0.8 g, 7.56 mmol, CAS RN 497-19-8) in water (4.2 mL), and the reaction mixture was stirred at 20° C. for 25 min. Then 2,4-dichlorobenzonitrile (2.0 g, 11.63 mmol, CAS RN 6574-98-7) was added and the mixture stirred at 95° C. for 12 h. The mixture was diluted with water, extracted with EtOAc, the organic layer dried over Na₂SO₄, filtered and concentrated. The residue was purified by reversed flash column chromatography (0.1% v/v FA in water and ACN) to give the desired product as light yellow solid (2 g, 83.9%). MS (ESI): m/z=205.5 [M+H]⁺.

### Step b) tert-Butyl 3-[[(Z)—C-(2,4-Dichlorophenyl)-N-hydroxy-carbonimidoyl]carbamoyl]azetidine-1-carboxylate

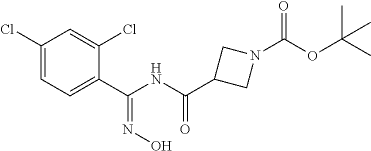 [SMILES: CC(C)(C)OC(=O)N1CC(C(=O)N/C(=N\O)c2ccc(Cl)cc2Cl)C1]

To a solution of 2,4-dichloro-N-hydroxy-benzamidine (400.0 mg, 1.95 mmol), 1-boc-azetidine-3-carboxylic acid (471.1 mg, 2.34 mmol) and HATU (890.1 mg, 2.34 mmol) in DCM (8 mL) was added DIPEA (756.4 mg, 5.85 mmol). The mixture was stirred at 20° C. for 16 h. The mixture was washed with water (40 mL), followed by brine (40 mL), the organic layer dried over Na₂SO₄, filtered and the filtrate concentrated to give the crude desired product as dark brown oil which was used directly in the next step (750 mg, 99%). MS (ESI): m/z=388.3 [M+H]⁺.

### Step c) tert-Butyl 3-[3-(2,4-Dichlorophenyl)-1,2,4-oxadiazol-5-yl]azetidine-1-carboxylate

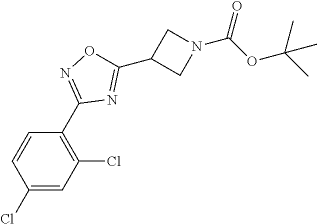 [SMILES: CC(C)(C)OC(=O)N1CC(c2nc(-c3ccc(Cl)cc3Cl)no2)C1]

A solution of tert-butyl 3-[[(Z)—C-(2,4-dichlorophenyl)-N-hydroxy-carbonimidoyl]carbamoyl] azetidine-1-carboxylate (750.0 mg, 1.93 mmol) in NMP (30 mL) was stirred at 130° C. for 12 h. The mixture was evaporated and the residue was purified by silica gel column (PE:EtOAc=10:1) to give the desired product as dark red oil (500 mg, 9.9%). MS (ESI): m/z=314.3 [M-56+H]⁺.

### Step d) 5-(Azetidin-3-yl)-3-(2,4-Dichlorophenyl)-1,2,4-oxadiazole (Trifluoroacetic Acid Salt)

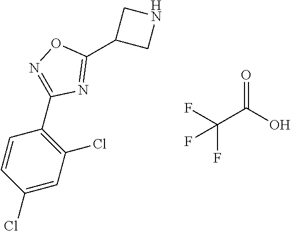 [SMILES: Clc1ccc(-c2noc(C3CNC3)n2)c(Cl)c1 ; O=C(O)C(F)(F)F]

To a solution of tert-butyl 3-[3-(2,4-dichlorophenyl)-1,2,4-oxadiazol-5-yl]azetidine-1-carboxylate (250.0 mg, 0.680 mmol) in DCM (2.5 mL) was added TFA (0.5 mL) and the mixture stirred at 20° C. for 12 h. The mixture was evaporated to give the crude desired product as dark brown oil which was used in the next step without further purification (253 mg, 97.5%). MS (ESI): m/z=270.4 [M+H].

### Example 17

### (4aR,8aS)-6-(3-(1-Methyl-1H-indazol-5-yl)azetidine-1-carbonyl)hexahydro-2H-pyrido[4,3-b][1,4]oxazin-3(4H)-one

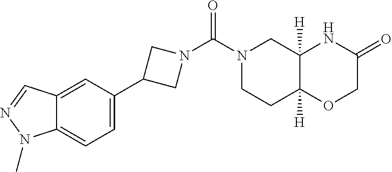 [SMILES: Cn1ncc2cc(C3CN(C(=O)N4CC[C@@H]5OCC(=O)N[C@@H]5C4)C3)ccc21]

To a suspension of 5-(azetidin-3-yl)-1-methyl-H-indazole 4-methylbenzenesulfonate (87 mg, 242 μmol) and 4-nitrophenyl (4aR,8aS)-3-oxohexahydro-2H-pyrido[4,3-b][1,4]oxazine-6(5H)-carboxylate (77.8 mg, 242 μmol) in ACN (708 μL) was added DIPEA (211 μL, 1.21 mmol) and the mixture was stirred at RT overnight. The solution was treated with silica gel and evaporated. The compound was purified by silica gel chromatography on a 4 g column using an MPLC (ISCO) system eluting with a gradient of n-heptane:EtOAc/EtOH 3/1 (70:30 to 10:90) followed by a second chromatography on silica gel on a 12 g column using an MPLC (ISCO) system eluting with a gradient of n-heptane:EtOAc/EtOH 3/1 (90:10 to 10:90) to provide the desired compound as a colorless foam (0.052 g; 58.1%). MS (ESI): m/z=370.2 [M+H]⁺.

### Step a) tert-Butyl 3-(I-methyl-H-indazol-5-yl)azetidine-1-carboxylate

The product was obtained in analogy to Example 10, step a, from 5-bromo-1-methyl-1H-indazole (CAS RN 465529-57-1) as a yellow oil. MS (ESI): m/z=288.2 [M+H]⁺.

### Step b) 5-(Azetidin-3-yl)-1-methyl-1H-indazole 4-methylbenzenesulfonate

A mixture of tert-butyl 3-(1-methyl-1H-indazol-5-yl)azetidine-1-carboxylate (70 mg, 244 μmol) and 4-methylbenzenesulfonic acid hydrate (55.6 mg, 292 μmol) in EtOAc (1 mL) was stirred at reflux for 30 min. The mixture was evaporated to yield the desired product which was used in the next step without further purification. MS (ESI): m/z=188.1 [M+H]⁺.

### Example 18

### (4aR,8aS)-6-(3-(5-(2,4-Dichlorophenyl)-1,2,4-oxadiazol-3-yl)azetidine-1-carbonyl)hexahydro-2H-pyrido[4,3-b][1,4]oxazin-3(4H)-one

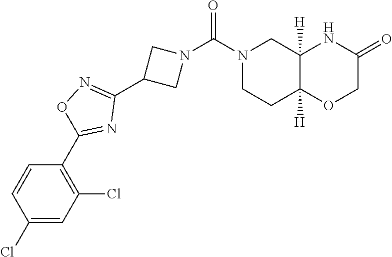 [SMILES: O=C1CO[C@H]2CCN(C(=O)N3CC(c4noc(-c5ccc(Cl)cc5Cl)n4)C3)C[C@H]2N1]

To a suspension of 3-(azetidin-3-yl)-5-(2,4-dichlorophenyl)-1,2,4-oxadiazole hydrochloride (48 mg, 157 μmol) and 4-nitrophenyl (4aR,8aS)-3-oxohexahydro-2H-pyrido[4,3-b][1,4]oxazine-6(5H)-carboxylate (50.3 mg, 157 μmol) in ACN (938 μL) was added DIPEA (80.9 mg, 109 μL, 626 μmol) and the mixture was stirred at RT overnight.

Then the reaction mixture was heated to 50° C. for one h. The yellow solution was evaporated and the product was purified by prep-HPLC (Gemini NX column) using a gradient of ACN:water (containing 0.1% formic acid) (20:80 to 98:2) to furnish the desired compound as a colorless gum (20 mg; 28.2%). MS (ESI): m/z=496.3 [M+HCOOH—H]⁻.

### Step a) (Z)-1-Benzhydryl-N′-hydroxyazetidine-3-carboximidamide

To 1-benzhydrylazetidine-3-carbonitrile (1.00 g, 4.03 mmol, CAS RN 36476-86-5) in EtOH (11.2 mL) were added hydroxylamine hydrochloride (392 mg, 5.64 mmol) and TEA (693 mg, 954 μL, 6.85 mmol) at RT. The reaction mixture was stirred and heated under reflux at 80° C. overnight. The mixture was concentrated and taken up in EtOAc and water. The aqueous layer was extracted with EtOAc and the organic layer was dried over Na₂SO₄ and concentrated to provide the product as a light brown solid (0.96 g, 84.7%). MS (ESI): m/z=282.2 [M+H]⁺.

### Step b) 3-(1-Benzhydrylazetidin-3-yl)-5-(2,4-dichlorophenyl)-1,2,4-oxadiazole

(Z)-1-benzhydryl-N′-hydroxyazetidine-3-carboximidamide (960 mg, 3.41 mmol) was dissolved in DMF (13.6 mL). Under argon, N-ethyl-N-isopropylpropan-2-amine (1.32 g, 1.74 mL, 10.2 mmol) was added and 2,4-dichlorobenzoyl chloride (715 mg, 478 μL, 3.41 mmol) was diluted with 0.5 mL DMF and added dropwise. The mixture was stirred at RT for 40 min., at 80° C. for 30 min. and at 100° C. for 6 h. The reaction mixture was mixed with EtOAc and water and the layers were separated. The aqueous layer was extracted twice with EtOAc. The organic layers were washed three times with water, dried over Na₂SO₄, filtered and evaporated with silica gel. The compound was purified on a 40 g column eluting with a gradient of n-heptane:EtOAc (100:0 to 50:50) to yield the desired compound as a colourless solid. (240 mg; 16.1%). MS (ESI): m/z=436.2 [M+H]⁺.

### Step c) 3-(Azetidin-3-yl)-5-(2,4-dichlorophenyl)-1,2,4-oxadiazole hydrochloride

In a vial, 3-(1-benzhydrylazetidin-3-yl)-5-(2,4-dichlorophenyl)-1,2,4-oxadiazole (120 mg, 275 μmol) was combined with DCM (0.75 mL) to give a slightly yellow solution. 1-Chloroethyl carbonochloridate (51.1 mg, 38.6 μL, 358 μmol) was added. The reaction mixture was heated to 70° C. and stirred for 1.5 h. After cooling to RT MeOH (0.75 mL) was added. The reaction mixture was heated to 70° C., stirred for 1 h and the light yellow solution was concentrated to dryness. The crude material was triturated with tert-butyl methyl ether and then filtered to yield the compound as a light yellow solid (50 mg, 59.3%). MS (ESI): m/z=270.1 [M+H]⁺.

### Example 19

### (4aR,8aS)-6-(3-(3-(Trifluoromethoxy)phenyl)azetidine-1-carbonyl)hexahydro-2H-pyrido[4,3-b][1,4]oxazin-3(4H)-one

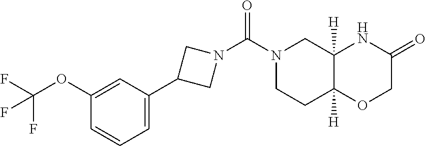 [SMILES: O=C1CO[C@H]2CCN(C(=O)N3CC(c4cccc(OC(F)(F)F)c4)C3)C[C@H]2N1]

To a suspension of 3-(3-(trifluoromethoxy)phenyl)azetidine 4-methylbenzenesulfonate (59 mg, 152 μmol) and 4-nitrophenyl (4aR,8aS)-3-oxohexahydro-2H-pyrido[4,3-b][1,4]oxazine-6(5H)-carboxylate (107 mg, 333 μmol) in ACN (0.5 mL) was added DIPEA (233 μL, 1.33 mmol) and the mixture was stirred at RT overnight. The yellow solution was evaporated and the crude product was purified by prep-HPLC (Gemini NX column) using a gradient of ACN:water (containing 0.1% TEA) (20:80 to 98:2) to furnish the desired compound as a colorless foam (0.045 g; 74.4%). MS (ESI): m/z=400.3 [M+H]⁺.

### Step a) tert-Butyl 3-(3-(trifluoromethoxy)phenyl)azetidine-1-carboxylate

To a stirred suspension of (3-(trifluoromethoxy)phenyl)boronic acid (291 mg, 1.41 mmol) in 2-propanol (2.5 mL) was added tert-butyl 3-iodoazetidine-1-carboxylate (200 mg, 706 μmol) at RT. To the mixture was added rac-(1R,2R)-2-aminocyclohexan-1-ol (4.88 mg, 42.4 μmol), nickel(II) iodide (13.2 mg, 42.4 μmol) and sodium bis(trimethylsilyl)amide 2 M in THF (706 μL, 1.41 mmol) under argon. The mixture was heated in a microwave oven at 80° C. for 1 h.

The reaction mixture was poured on water and EtOAc and the layers were separated. The aqueous layer was extracted twice with EtOAc. The organic layers were dried over MgSO₄, filtered, treated with silica gel and evaporated. The crude compound was purified by silica gel chromatography on a 4 g column using an MPLC system eluting with a gradient of n-heptane:EtOAc (100:0 to 50:50) to give the desired compound as a colorless oil (0.129 g; 40.3%). MS (ESI): m/z=262.1 [M-56+H]⁺.

### Step b) 3-(3-(Trifluoromethoxy)phenyl)azetidine 4-methylbenzenesulfonate

A solution of tert-butyl 3-(3-(trifluoromethoxy)phenyl)azetidine-1-carboxylate (129 mg, 407 μmol) and 4-methylbenzenesulfonic acid monohydrate (92.8 mg, 488 μmol) in EtOAc (2 mL) was stirred at reflux for 1 h. The mixture was allowed to cool down to RT, then cooled in an ice-bath and filtered. The filter cake was washed with a small volume of EtOAc to give the desired compound as a colorless solid (0.061 g; 38.5%). MS (ESI): m/z=218.1 [M+H]⁺.

### Example 20

### (4aR,8aS)-6-(4-(1-(4-Fluorophenyl)-1H-pyrazol-3-yl)piperidine-1-carbonyl)hexahydro-2H-pyrido[4,3-b][1,4]oxazin-3(4H)-one

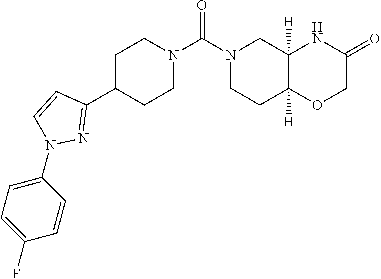 [SMILES: O=C1CO[C@H]2CCN(C(=O)N3CCC(c4ccn(-c5ccc(F)cc5)n4)CC3)C[C@H]2N1]

Example 20 was synthesized as described for Example 47, starting from (4aR,8aS)-hexahydro-2H-pyrido[4,3-b][1,4]oxazin-3(4H)-one (BB1a) and 4-(1-(4-fluorophenyl)-1H-pyrazol-3-yl)piperidine hydrochloride. The product was purified by preparative HPLC (Gemini NX, 12 nm, 5 μm, 100×30 mm, gradient ACN in water+0.1% HCOOH). MS (ESI): m/z=428.3 [M+H]⁺.

### Step a) tert-butyl 4-(1-(4-fluorophenyl)-1H-pyrazol-3-yl)piperidine-1-carboxylate

In a 100 ml glas flask under argon, tert-butyl 4-(1H-pyrazol-3-yl)piperidine-1-carboxylate (CAS 278798-07-5, 300 mg, 1.19 mmol) was suspended in DMF (8 ml), pyridine (378 mg, 386 μl, 4.77 mmol), (4-fluorophenyl)boronic acid (217 mg, 1.55 mmol) and copper (II) acetate (325 mg, 1.79 mmol) were added, the green solution was stirred 60 hr at RT. The solvent was removed in vacuo, the residue was extracted with ethyl acetat/water/sat. NaCl, organic fractions were dried over MgSO₄, removing the solvent in vacuo and chromatography (20 g silica, heptane/EA 0 to 40% in 40 min) yielded the desired product as a colorless viscouos oil (290 mg, 70%). MS (ESI): m/z=290.2 [M-56+H]⁺.

### Step b) 4-(1-(4-fluorophenyl)-1H-pyrazol-3-yl)piperidine hydrochloride

Deprotection was achieved in analogy to example 47, step b. MS (ESI): m/z=246.2 [M+H]⁺.

### Example 23

### (4aR,8aS)-6-(4-((5-Cyclopropyl-4-methylpyridin-3-yl)methyl)piperidine-1-carbonyl)hexahydro-2H-pyrido[4,3-b][1,4]oxazin-3(4H)-one

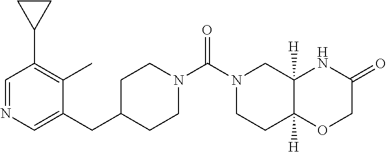 [SMILES: Cc1c(CC2CCN(C(=O)N3CC[C@@H]4OCC(=O)N[C@@H]4C3)CC2)cncc1C1CC1]

Example 23 was synthesized as described for Example 47, starting from (4aR,8aS)-hexahydro-2H-pyrido[4,3-b][1,4]oxazin-3(4H)-one (BB1a) and 3-cyclopropyl-4-methyl-5-(piperidin-4-ylmethyl)pyridine dihydrochloride. The product was purified by preparative HPLC (Gemini NX, 12 nm, 5 μm, 100×30 mm, gradient ACN in water+0.1% TEA). MS (ESI): m/z=413.2 [M+H]⁺.

### Step a) tert-Butyl 4-((5-cyclopropyl-4-methylpyridin-3-yl)methyl)piperidine-1-carboxylate

Synthesis was performed as described for example 37, step a, starting from tert-butyl 4-methylenepiperidine-1-carboxylate and 3-bromo-5-cyclopropyl-4-methylpyridine. MS (ESI): m/z=331.3 [M+H]⁺.

### Step b) 3-Cyclopropyl-4-methyl-5-(piperidin-4-ylmethyl)pyridine dihydrochloride

Deprotection was achieved in analogy to example 47, step b. MS (ESI): m/z=231.4 [M+H]⁺.

### Examples 27, 28, 29

### (4aR,8aS)-6-[3-[[2-Fluoro-6-(trifluoromethyl)phenyl]methoxy]-2-methyl-azetidine-1-carbonyl]-4,4a,5,7,8,8a-hexahydropyrido[4,3-b][1,4]oxazin-3-one

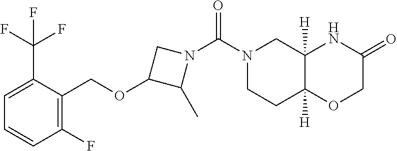 [SMILES: CC1C(OCc2c(F)cccc2C(F)(F)F)CN1C(=O)N1CC[C@@H]2OCC(=O)N[C@@H]2C1]

A round-bottom flask was heat gun-dried under high vacuum, back filled with argon and charged with bis(trichloromethyl) carbonate (133 mg, 448 μmol) and sodium bicarbonate (215 mg, 2.56 mmol). DCM (2 ml) was added to give a suspension. (4aR,8aS)-hexahydro-2H-pyrido[4,3-b][1,4]oxazin-3(4H)-one (BB1a, 0.100 g, 640 μmol) was added to the suspension at 0° C. The mixture was stirred at 0° C. for 5 min and at RT for 20 hours. 3-((2-fluoro-6-(trifluoromethyl)benzyl)oxy)-2-methylazetidine trifluoroacetate (242 mg, 640 μmol) and DIPEA (331 mg, 447 μl, 2.56 mmol) were added. The resulting off-white suspension was stirred at RT for 1 hour. The reaction mixture was poured into 5 mL H₂O and extracted with DCM (2×10 mL). The organic layers were combined, washed with brine, dried over Na₂SO₄ and concentrated in vacuo. The crude material was purified by flash chromatography (silica gel 20 g, 0% to 10% MeOH in DCM) fractions were combined and evaporated, yielding 147 mg (52%) of a colorless oil. Subsequent chiral HPLC (ReprosilChiral NR, 70% Heptane, 30% Ethanol+0.1% NH₄OAc) yielded the separate diastereomers:

### Example 27

First eluting peak, diastereomers A+B, 57 mg, MS (ESI): m/z=446.3 [M+H]⁺.

### Example 28

Second eluting peak, diastereomer C, 29 mg, MS (ESI): m/z=446.3 [M+H]⁺.

### Example 29

Third eluting peak, diastereomer D, 25 mg, MS (ESI): m/z=446.3 [M+H]⁺.

### Step a) tert-butyl 3-((2-fluoro-6-(trifluoromethyl)benzyl)oxy)-2-methylazetidine-1-carboxylate

In a 25 mL two-necked flask, tert-butyl 3-hydroxy-2-methylazetidine-1-carboxylate (200 mg, 1.07 mmol) was combined with DMF (5 ml) to give a colorless solution. At 0° C., sodium hydride 60% dispersion in mineral oil (38.9 mg, 973 μmol) was added. The reaction mixture was stirred at 0° C. for 15 min. Then 1-(bromomethyl)-2-fluoro-4-(trifluoromethyl)benzene (0.25 g, 973 μmol) was added at 0° C. The reaction mixture was stirred at RT for 5 hours. The reaction mixture was poured into 20 mL sat NH₄Cl and extracted with EtOAc (2×50 mL). The organic layers were combined, washed with brine, dried over Na₂SO₄ and concentrated in vacuo. Used directly without purification for the next step. MS (ESI): m/z=308.1 [M-56+H]⁺.

### Step b) 3-((2-fluoro-6-(trifluoromethyl)benzyl)oxy)-2-methylazetidine trifluoroacetate

tert-butyl 3-((4-(pentafluoro-16-sulfanyl)benzyl)oxy)azetidine-1-carboxylate (0.493 g, 1.36 mmol) was dissolved in DCM (5 ml) and TFA (1.24 g, 836 μl, 10.9 mmol) was added. The reaction mixture was stirred overnight at RT and concentrated in vacuo (azeotrop with toluol). Used directly in next step. MS (ESI): m/z=264.2 [M+H]⁺.

### Example 30

### (4aR,8aS)-6-(3-((4-Fluoro-3-methoxybenzyl)oxy)azetidine-1-carbonyl)hexahydro-2H-pyrido[4,3-b][1,4]oxazin-3(4H)-one

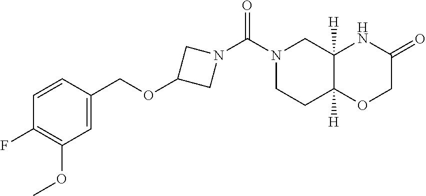 [SMILES: COc1cc(COC2CN(C(=O)N3CC[C@@H]4OCC(=O)N[C@@H]4C3)C2)ccc1F]

Example 30 was prepared in analogy as described for example 16, starting from 4-nitrophenyl (4aR,8aS)-3-oxohexahydro-2H-pyrido[4,3-b][1,4]oxazine-6(5H)-carboxylate (BB2a) and 3-((4-fluoro-3-methoxybenzyl)oxy)azetidine trifluoroacetate. Purification by preparative HPLC: Gemini NX, 12 nm, 5 μm, 100×30 mm, gradient ACN/Water+0.1% TEA). MS (ESI): m/z=394.3 [M+H]⁺.

### Step a) 3-((4-fluoro-3-methoxybenzyl)oxy)azetidine trifluoroacetate

Prepared as described for Example 31, steps a and b. MS (ESI): m/z=212.2 [M+H]⁺.

### Example 31

### (4aR,8aS)-6-(3-((4-(Pentafluoro-16-sulfanyl)benzyl)oxy)azetidine-1-carbonyl)hexahydro-2H-pyrido[4,3-b][1,4]oxazin-3(4H)-one

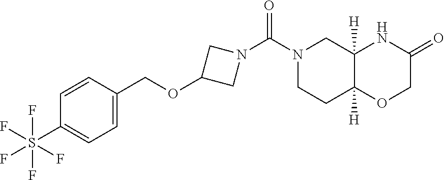 [SMILES: O=C1CO[C@H]2CCN(C(=O)N3CC(OCc4ccc(S(F)(F)(F)(F)F)cc4)C3)C[C@H]2N1]

A round-bottom flask was heat gun-dried under HV, back filled with argon and charged with bis(trichloromethyl) carbonate (39.9 mg, 134 μmol) and sodium bicarbonate (64.5 mg, 768 μmol). DCM (2 ml) was added to give a suspension. (4aR,8aS)-hexahydro-2H-pyrido[4,3-b][1,4]oxazin-3(4H)-one (BB1a, 0.03 g, 192 μmol) was added to the suspension at 0° C. The mixture was stirred at 0° C. for 5 min and at RT for 20 hours.

3-((4-(pentafluoro-16-sulfanyl)benzyl)oxy)azetidine trifluoroacetate (77.5 mg, 192 μmol) and DIPEA (99.3 mg, 134 μl, 768 μmol) were added. The resulting off-white suspension was stirred at RT for 1 hour. The reaction mixture was poured into 5 mL H₂O and extracted with DCM (2×10 mL). The organic layers were combined, washed with brine, dried over Na₂SO₄ and concentrated in vacuo. The crude material was purified by flash chromatography (silica gel, 10 g, 0% to 10% MeOH in DCM). Fractions were combined and evaporated to yield 53.1 mg (59%) of desired product as a white foam. MS (ESI): m/z=472.13 [M+H]⁺.

### Step a) tert-butyl 3-((4-(pentafluoro-16-sulfanyl)benzyl)oxy)azetidine-1-carboxylate

To a solution of tert-butyl 3-hydroxyazetidine-1-carboxylate (200 mg, 1.15 mmol) in dry THF (3 ml) was added potassium tert-butoxide 1M solution in THF (1.21 ml, 1.21 mmol) and the turbid reaction mixture was stirred at r.t for 15 min followed by addition of (4-(bromomethyl)phenyl)pentafluoro-16-sulfane (343 mg, 1.15 mmol). The reaction mixture was then stirred at r.t for 19 hours. The crude reaction was diluted with ethyl acetate and extracted with water, the organic phase was collected and the aqueous phase was back-extracted with ethyl acetate. The combined organic phases were dried over sodium sulfate and evaporated down to dryness to yield 476 mg of the crude product as a light yellow oil which was used without further purification. MS (ESI): m/z=334.1 [M-56+H]⁺.

### Step b) 3-((4-(pentafluoro-16-sulfanyl)benzyl)oxy)azetidine trifluoroacetate

tert-butyl 3-((4-(pentafluoro-16-sulfanyl)benzyl)oxy)azetidine-1-carboxylate (0.476 g, 1.22 mmol) was dissolved in DCM (5 ml) and TFA (1.12 g, 753 μl, 9.78 mmol) was added. The reaction mixture was stirred overnight at RT and concentrated in vacuo (azeotrop with toluol). Used directly in next step. MS (ESI): m/z=290.1 [M+H]⁺.

The following examples were all prepared in analogy to Example 31

### Example 21

### (4aR,8aS)-6-(3-((2,6-Dichlorobenzyl)oxy)azetidine-1-carbonyl)hexahydro-2H-pyrido[4,3-b][1,4]oxazin-3(4H)-one

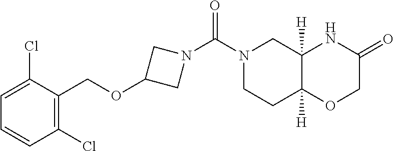 [SMILES: O=C1CO[C@H]2CCN(C(=O)N3CC(OCc4c(Cl)cccc4Cl)C3)C[C@H]2N1]

Prepared starting from tert-butyl 3-hydroxyazetidine-1-carboxylate, 2-(bromomethyl)-1,3-dichlorobenzene and BB1a. MS (ESI): m/z=414.09 [M+H]⁺.

### Example 22

### (4aR,8aS)-6-(3-((3,5-Dichlorobenzyl)oxy)azetidine-1-carbonyl)hexahydro-2H-pyrido[4,3-b][1,4]oxazin-3(4H)-one

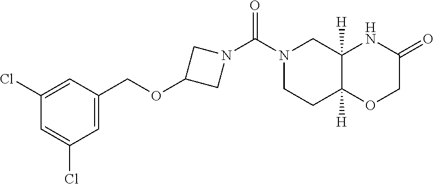 [SMILES: O=C1CO[C@H]2CCN(C(=O)N3CC(OCc4cc(Cl)cc(Cl)c4)C3)C[C@H]2N1]

Prepared starting from tert-butyl 3-hydroxyazetidine-1-carboxylate, 1-(bromomethyl)-3,5-dichlorobenzene and BB1a. MS (ESI): m/z=414.10 [M+H]⁺.

### Example 24

### (4aR,8aS)-6-(4-((4-(Trifluoromethyl)benzyl)oxy)piperidine-1-carbonyl)hexahydro-2H-pyrido[4,3-b][1,4]oxazin-3(4H)-one

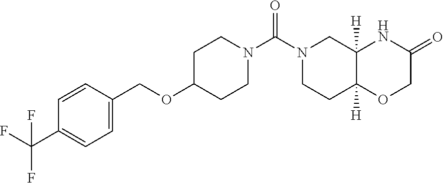 [SMILES: O=C1CO[C@H]2CCN(C(=O)N3CCC(OCc4ccc(C(F)(F)F)cc4)CC3)C[C@H]2N1]

Prepared starting from tert-butyl 4-hydroxypiperidine-1-carboxylate, 1-(bromomethyl)-4-(trifluoromethyl)benzene and BB1a. MS (ESI): m/z=441.19 [M+H]⁺.

### Example 25

### (4aR,8aS)-6-(4-((2-Chloro-4-fluorobenzyl)oxy)piperidine-1-carbonyl)hexahydro-2H-pyrido[4,3-b][1,4]oxazin-3(4H)-one

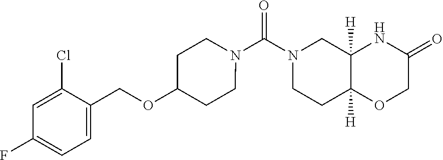 [SMILES: O=C1CO[C@H]2CCN(C(=O)N3CCC(OCc4ccc(F)cc4Cl)CC3)C[C@H]2N1]

Prepared starting from tert-butyl 4-hydroxypiperidine-1-carboxylate, 1-(bromomethyl)-2-chloro-4-fluorobenzene and BB1a. MS (ESI): m/z=426.16 [M+H]⁺.

### Example 26

### (4aR,8aS)-6-(4-((2-chloro-4-(trifluoromethyl)benzyl)oxy)piperidine-1-carbonyl)hexahydro-2H-pyrido[4,3-b][1,4]oxazin-3(4H)-one

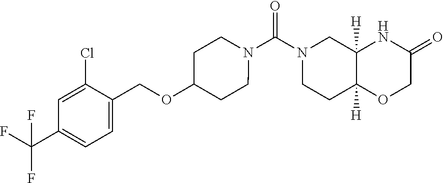 [SMILES: O=C1CO[C@H]2CCN(C(=O)N3CCC(OCc4ccc(C(F)(F)F)cc4Cl)CC3)C[C@H]2N1]

Prepared starting from tert-butyl 4-hydroxypiperidine-1-carboxylate, 1-(bromomethyl)-2-chloro-4-(trifluoromethyl)benzene and BB1a. MS (ESI): m/z=476.16 [M+H]⁺.

### Example 32

### (4aR,8aS)-6-(3-((2-(Trifluoromethoxy)benzyl)oxy)azetidine-1-carbonyl)hexahydro-2H-pyrido[4,3-b][1,4]oxazin-3(4H)-one

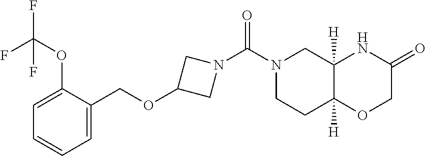 [SMILES: O=C1CO[C@H]2CCN(C(=O)N3CC(OCc4ccccc4OC(F)(F)F)C3)C[C@H]2N1]

Prepared starting from tert-butyl 3-hydroxyazetidine-1-carboxylate, 1-(bromomethyl)-2-(trifluoromethoxy)benzene and BB1a. MS (ESI): m/z=430.16 [M+H]⁺.

### Example 33

### (4aR,8aS)-6-(3-((4-Chloro-2-(trifluoromethoxy)benzyl)oxy)azetidine-1-carbonyl)hexahydro-2H-pyrido[4,3-b][1,4]oxazin-3(4H)-one

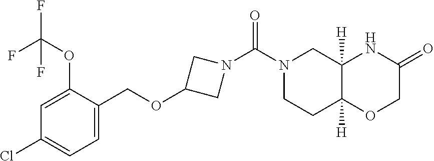 [SMILES: O=C1CO[C@H]2CCN(C(=O)N3CC(OCc4ccc(Cl)cc4OC(F)(F)F)C3)C[C@H]2N1]

Prepared starting from tert-butyl 3-hydroxyazetidine-1-carboxylate, 1-(bromomethyl)-4-chloro-2-(trifluoromethoxy)benzene and BB1a. MS (ESI): m/z=464.12 [M+H]⁺.

### Example 34

### (4aR,8aS)-6-(3-((2-Methyl-5-(trifluoromethyl)benzyl)oxy)azetidine-1-carbonyl)hexahydro-2H-pyrido[4,3-b][1,4]oxazin-3(4H)-one

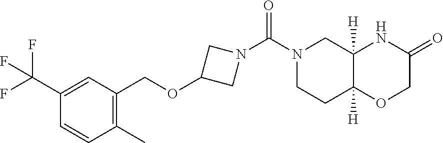 [SMILES: Cc1ccc(C(F)(F)F)cc1COC1CN(C(=O)N2CC[C@@H]3OCC(=O)N[C@@H]3C2)C1]

Prepared starting from tert-butyl 3-hydroxyazetidine-1-carboxylate, 2-(bromomethyl)-1-methyl-4-(trifluoromethyl)benzene and BB1a. MS (ESI): m/z=422.18 [M+H]⁺.

### Example 35

### (4aR,8aS)-6-[4-[[3-Phenyl-4-(trifluoromethyl)phenyl]methyl]piperidine-1-carbonyl]-4,4a,5,7,8,8a-hexahydropyrido[4,3-b][1,4]oxazin-3-one

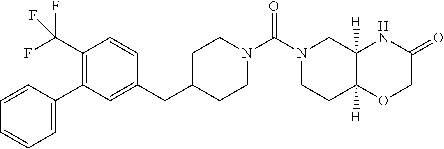 [SMILES: O=C1CO[C@H]2CCN(C(=O)N3CCC(Cc4ccc(C(F)(F)F)c(-c5ccccc5)c4)CC3)C[C@H]2N1]

A mixture of 4-[[3-phenyl-4-(trifluoromethyl)phenyl]methyl]piperidine; hydrochloride salt (110.8 mg, 0.310 mmol), (4-nitrophenyl) (4aR,8aS)-3-oxo-4,4a,5,7,8,8a-hexahydropyrido[4,3-b][1,4]oxazine-6-carboxylate (100.0 mg, 0.310 mmol) and DIEA (120.59 mg, 0.930 mmol) in ACN (2 mL) was stirred at 80° C. for 12 h. The mixture was evaporated and the residue purified by prep-HPLC (0.225% v/v FA in water and ACN) to give the desired compound as off-white solid (75.6 mg, 48.4%). MS (ESI): m/z=502.1 [M+H]⁺.

### Step a) 4-Methyl-2-phenyl-1-(trifluoromethyl)benzene

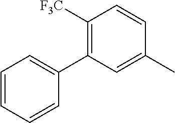 [SMILES: Cc1ccc(C(F)(F)F)c(-c2ccccc2)c1]

A mixture of phenylboronic acid (1.84 g, 15.06 mmol, CAS RN 98-80-6), 2-bromo-4-methyl-1-(trifluoromethyl)benzene (3.0 g, 12.55 mmol, CAS RN 121793-12-2), potassium carbonate (3.47 g, 25.1 mmol) and Pd(PPh₃)4 (1.45 g, 1.26 mmol) in DMF (30 mL) and water (3 mL) was stirred at 110° C. for 12 h. The mixture was poured into water (100 mL) and extracted twice with EtOAc (100 mL each). The combined organic layers were washed with brine (100 mL), dried over Na₂SO₄, filtered and concentrated. The residue was purified by column chromatography with PE as eluant to give the title compound as colorless oil (2.85 g, 96.1%).

### Step b) 4-(Bromomethyl)-2-phenyl-1-(trifluoromethyl)benzene

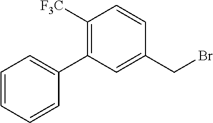 [SMILES: FC(F)(F)c1ccc(CBr)cc1-c1ccccc1]

A mixture of 4-methyl-2-phenyl-1-(trifluoromethyl)benzene (2.85 g, 12.06 mmol), NBS (2241.4 mg, 12.7 mmol) and benzoyl peroxide (394.5 mg, 1.63 mmol) in carbon tetrachloride (30 mL) was stirred at 70° C. for 12 h. The mixture was poured into water (50 mL), and extracted twice with DCM (50 mL each). The combined organic layers were washed with brine (30 mL), dried over Na₂SO₄, filtered and concentrated over vacuum to give the crude product as light yellow oil which was used in the next step without further purification.

### Step c) 4-(Diethoxyphosphorylmethyl)-2-phenyl-1-(trifluoromethyl)benzene

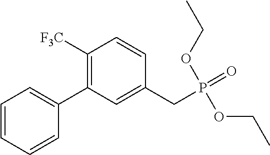 [SMILES: CCOP(=O)(Cc1ccc(C(F)(F)F)c(-c2ccccc2)c1)OCC]

A mixture of 4-(bromomethyl)-2-phenyl-1-(trifluoromethyl)benzene (4.80 g, crude) in triethyl phosphite (20.0 mL) was stirred at 155° C. for 5 h. The mixture was concentrated under vacuum and the residue purified by column chromatography (PE:EtOAc=1:0 to 3:1) to give the crude product as light yellow oil.

### Step d) tert-Butyl 4-[[3-phenyl-4-(trifluoromethyl)phenyl]methylene]piperidine-1-carboxylate

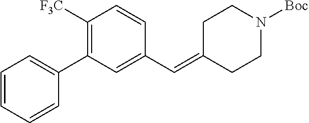 [SMILES: CC(C)(C)OC(=O)N1CCC(=Cc2ccc(C(F)(F)F)c(-c3ccccc3)c2)CC1]

To a mixture of 4-(diethoxyphosphorylmethyl)-2-phenyl-1-(trifluoromethyl)benzene (2.0 g, 5.37 mmol) in THF (20 mL) was added sodium hydride (322.3 mg, 8.06 mmol) protionwise at 0° C. The mixture was stirred at 20° C. for 1 h, then 1-boc-4-piperidone (1605.5 mg, 8.06 mmol) was added and the mixture was stirred at 20° C. for 12 h. The mixture was poured into water (50 mL) and extracted three times with EtOAc (50 mL each). The combined organic layers were washed twice with brine (50 mL each), dried over Na₂SO₄, filtered and concentrated under vacuum. The residue was purified by column chromatography (PE:EtOAc=50:1) to give the title compound as colorless gum (670 mg, 29.9%). MS (ESI): m/z=362.0 [M-56+H]⁺.

### Step e) tert-Butyl 4-[[3-phenyl-4-(trifluoromethyl)phenyl]methyl]piperidine-1-carboxylate

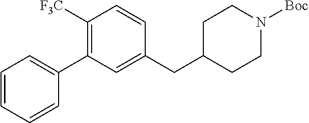 [SMILES: CC(C)(C)OC(=O)N1CCC(Cc2ccc(C(F)(F)F)c(-c3ccccc3)c2)CC1]

A mixture of tert-butyl 4-[[3-phenyl-4-(trifluoromethyl)phenyl]methylene]piperidine-1-carboxylate (670.0 mg, 1.6 mmol) and Pd/C (wt. 10%, 70.0 mg) in EtOAc (10 mL) was stirred at 20° C. for 12 h under H₂ atmosphere (1520 mmHg). The mixture was filtered and the filtrate concentrated under vacuum to give the desired compound as colorless gum (650 mg, 96.6%). MS (ESI): m/z=364.1 [M-56+H]⁺.

### Step f) 4-[[3-Phenyl-4-(trifluoromethyl)phenyl]methyl]piperidine hydrochloride salt

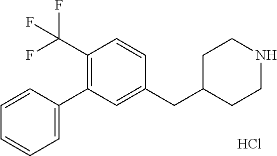 [SMILES: FC(F)(F)c1ccc(CC2CCNCC2)cc1-c1ccccc1 ; Cl]

A mixture of tert-butyl 4-[[3-phenyl-4-(trifluoromethyl)phenyl]methyl]piperidine-1-carboxylate (650.0 mg, 1.55 mmol) in HCl/dioxane (4M, 10 mL) was stirred at 20° C. for 12 h. The mixture was concentrated under vacuum to give the title compound as light yellow solid (550 mg, 99.8%). MS (ESI): m/z=320.2 [M+H]⁺.

### Example 36

### (4aR,8aS)-6-[4-[[2,4-bis(Trifluoromethyl)phenyl]methyl]piperidine-1-carbonyl]-4,4a,5,7,8,8a-hexahydropyrido[4,3-b][1,4]oxazin-3-one

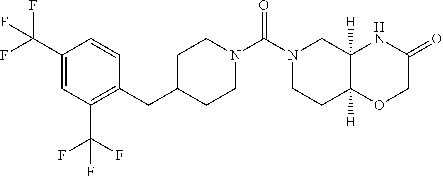 [SMILES: O=C1CO[C@H]2CCN(C(=O)N3CCC(Cc4ccc(C(F)(F)F)cc4C(F)(F)F)CC3)C[C@H]2N1]

A solution of 4-[[2,4-bis(trifluoromethyl)phenyl]methyl]piperidine formic acid salt (100.0 mg, 0.280 mmol) and (4-nitrophenyl) (4aR,8aS)-3-oxo-4,4a,5,7,8,8a-hexahydropyrido[4,3-b][1,4]oxazine-6-carboxylate (107.91 mg, 0.340 mmol) in ACN (5 mL) was added DIPEA (108.3 mg, 0.840 mmol). The mixture was stirred at 80° C. for 15 h. The mixture was concentrated and the residue was purified by reverse flash column chromatography (0.1% v/v FA in water and ACN) to give the title compound (20.3 mg, 14.5%) as white solid. MS (ESI): m/z=494.2 [M+H]⁺.

### Step a) 1-(Diethoxyphosphorylmethyl)-2,4-bis(trifluoromethyl)benzene

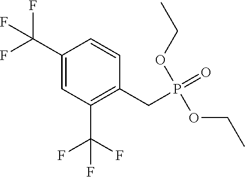 [SMILES: CCOP(=O)(Cc1ccc(C(F)(F)F)cc1C(F)(F)F)OCC]

A solution of 2,4-bis(trifluoromethyl)benzyl bromide (1.29 mL, 6.51 mmol, CAS RN 140690-56-8) in triethyl phosphite (10.82 g, 65.14 mmol, CAS RN 122-52-1) was stirred at 160° C. for 5 h. The mixture was filtered and concentrated under vacuum to give the title compound (2.27 g, 5.7%) as colorless oil.

### Step b) tert-Butyl 4-[[2,4-bis(trifuoromethyl)phenyl]methylene]piperidine-1-carboxylate

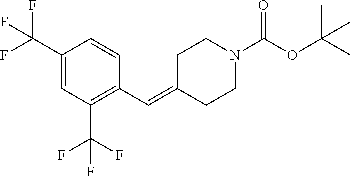 [SMILES: CC(C)(C)OC(=O)N1CCC(=Cc2ccc(C(F)(F)F)cc2C(F)(F)F)CC1]

A mixture of 1-(diethoxyphosphorylmethyl)-2,4-bis(trifluoromethyl)benzene (2.2 g, 6.04 mmol) in THF (10 mL) was added to sodium hydride (0.72 g, 18.12 mmol) in THF (10 mL) at 0° C. The mixture was stirred at 0° C. for 1 h, then 1-boc-4-piperidone (2.41 g, 12.1 mmol) was added and the mixture was stirred at 20° C. for 12 h. The mixture was poured into water (100 mL) and extracted three times with EtOAc (100 mL each). The combined organic layers were washed with brine (100 mL), dried over Na₂SO₄, filtered and concentrated under vacuum. The residue was purified by column chromatography (PE:EtOAc=50:1) to give the title compound as yellow oil (2.1 g, 48.0%). MS (ESI): m/z=354.0 [M-56+H]⁺.

### Step c) tert-Butyl 4-[[2,4-bis(trifluoromethyl)phenyl]methyl]piperidine-1-carboxylate

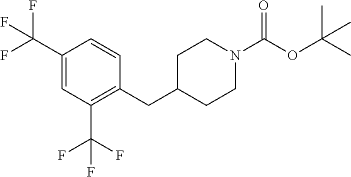 [SMILES: CC(C)(C)OC(=O)N1CCC(Cc2ccc(C(F)(F)F)cc2C(F)(F)F)CC1]

A mixture of tert-butyl 4-[[2,4-bis(trifluoromethyl)phenyl]methylene]piperidine-1-carboxylate (1.0 g, 2.44 mmol) in EtOAc (10 mL) was added Pd\C (100.0 mg, 0.240 mmol). The mixture was stirred at 20° C. under H₂ atmosphere for 12 h. The mixture was filtered and concentrated to give the title compound as light grey oil which was used in the next step without further purification (1 g, 99.5%).

### Step d) 4-[[2,4-Bis(trifluoromethyl)phenyl]methyl]piperidine(Formic Acid Salt)

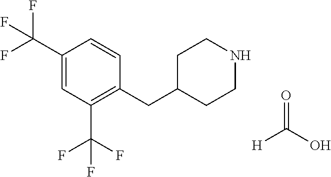 [SMILES: FC(F)(F)c1ccc(CC2CCNCC2)c(C(F)(F)F)c1 ; O=CO]

A solution of tert-butyl 4-[[2,4-bis(trifluoromethyl)phenyl]methyl]piperidine-1-carboxylate (990.0 mg, 2.41 mmol) in HCl\dioxane (4M, 20.0 mL) was stirred at 20° C. for 1 h.

The mixture was concentrated, and the residue was re-dissolved in water (100 mL), washed three times with EtOAc (30 mL each). The layers were separated and the water phase was lyophilized to give the crude product, which was purified by reverse flash column chromatography (0.1% v/v FA in water and ACN) to give the title compound as brown oil (134.7 mg, 15.4%). MS (ESI): m/z=312.0 [M+H]⁺.

### Example 37

### (4aR,8aS)-6-(4-((5-Methyl-6-(trifluoromethyl)pyridin-3-yl)methyl)piperidine-1-carbonyl)hexahydro-2H-pyrido[4,3-b][1,4]oxazin-3(4H)-one

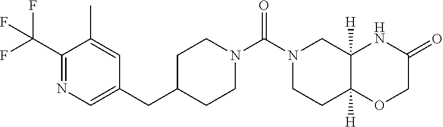 [SMILES: Cc1cc(CC2CCN(C(=O)N3CC[C@@H]4OCC(=O)N[C@@H]4C3)CC2)cnc1C(F)(F)F]

Example 37 was synthesized as described for Example 47, starting from (4aR,8aS)-hexahydro-2H-pyrido[4,3-b][1,4]oxazin-3(4H)-one (BB1a) and 3-methyl-5-(piperidin-4-ylmethyl)-2-(trifluoromethyl)pyridine dihydrochloride. The product was purified by preparative HPLC (Gemini NX, 12 nm, 5 μm, 100×30 mm, gradient ACN in water+0.1% TEA). MS (ESI): m/z=441.3 [M+H]⁺.

### Step a) tert-butyl 4-((5-methyl-6-(trifluoromethyl)pyridin-3-yl)methyl)piperidine-1-carboxylate

Under Ar, tert-butyl 4-methylenepiperidine-1-carboxylate (500 mg, 2.53 mmol) was diluted in degased THF (9 ml). 9-borabicyclo[3.3.1]nonane 0.5M in THF (5.58 ml, 2.79 mmol) was added and the reaction mixture was stirred at 66° C. for 2 hr. At RT, this colorless solution was added to an orange degassed solution containing 5-bromo-3-methyl-2-(trifluoromethyl)pyridine (608 mg, 2.53 mmol), PdCl₂(DPPF) complex with DCM (103 mg, 127 μmol) and potassium carbonate (420 mg, 3.04 mmol) in DMF (9 ml) and water (603 μl). The reaction mixture was stirred at 66° C. for 17 hr. The reaction mixture was diluted with EA and washed with water (3×) sat.NaCl (1×), dried over magnesium sulfate and concentrated to dryness. The residue was purified by flash chromatography (50 g SiO₂, 0-35% EA in heptane in 40 min), yielding the desired product as a colorless viscouos oil (908 mg, 78%). MS (ESI): m/z=303.2 [M-56+H]⁺.

### Step b) 3-methyl-5-(piperidin-4-ylmethyl)-2-(trifluoromethyl)pyridine dihydrochloride

Deprotection was achieved in analogy to example 47, step b. MS (ESI): m/z=259.2 [M+H]⁺.

### Example 38 and Example 39

### (4aR,8aS)-6-[rel-(3R,4R)-4-[(2-chloro-4-fluoro-phenoxy)methyl]-3-methyl-piperidine-1-carbonyl]-4,4a,5,7,8,8a-hexahydropyrido[4,3-b][1,4]oxazin-3-one

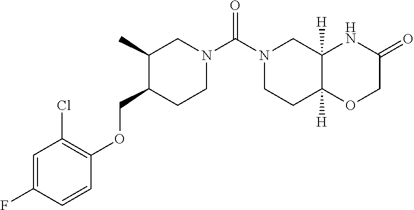 [SMILES: C[C@H]1CN(C(=O)N2CC[C@@H]3OCC(=O)N[C@@H]3C2)CC[C@H]1COc1ccc(F)cc1Cl]

Examples 38 and 39 was synthesized as described for Example 47, starting from (4aR,8aS)-hexahydro-2H-pyrido[4,3-b][1,4]oxazin-3(4H)-one acetate (BB1a) and rel-(3R,4R)-4-((2-chloro-4-fluorophenoxy)methyl)-3-methylpiperidine hydrochloride. The product was purified by preparative HPLC (YMC-Triart C18, 12 nm, 5 μm, 100×30 mm, 9 min gradient 40-60-80-100% ACN in water+0.1% TEA). The two diastereomers were separated by chiral HPLC (Chiralcel OD, 35 ml/min, 60% heptane, 40% ethanol+0.1% NH4OAc).

Example 38: First eluting diastereomer, MS (ESI): m/z=440.3 [M-56+H]⁺.

Example 39: Second eluting diastereomer, MS (ESI): m/z=440.3 [M-56+H]⁺.

### Step a) tert-butyl rel-(3R,4R)-4-(hydroxymethyl)-3-methylpiperidine-1-carboxylate

To a stirred solution of cis-N-Boc-3-methylpiperidine-4-carboxylic acid methyl ester (2 g, 7.77 mmol) in THF (10 ml) was added lithium borohydride (5.83 ml, 11.7 mmol) at 2-5° C. The reaction mixture was then heated at reflux for 3 hr and then cooled to 2-5° C. 10 ml water was added and the aqueous layer was extracted with ethyl acetate (2×30 ml). The organic layer was washed with water, 10 ml NaHCO₃ and 10 ml brine, the layers were separated, and the organics dried over Na₂SO₄, concentrated in vacuum. Purification by flash chromatography over 50 g column with heptane (EA 0 to 65% in 60 min) yielded the desired product as a colorless oil (844 mg, 47%). MS (ESI): m/z=174.1 [M-56+H]⁺.

### Step b) tert-butyl rel-(3R,4R)-4-((2-chloro-4-fluorophenoxy)methyl)-3-methylpiperidine-1-carboxylate

In a 50 ml four-necked sulphonation flask under argon, tert-butyl rel-(3R,4R)-4-(hydroxymethyl)-3-methylpiperidine-1-carboxylate (840 mg, 3.66 mmol) was dissolved in THF (15 ml) and 2-chloro-4-fluorophenol (590 mg, 439 μl, 4.03 mmol) and triphenylphosphine (1.06 g, 4.03 mmol) were added, the clear solution was stirred 5 min at RT, then cooled to 0-2° C. and slowly DEAD (702 mg, 638 μl, 4.03 mmol) was added within in 10 min, 1 hr stirred at 2-4° C. and removed the cooling bath, stirred over night at RT. 50 ml diethylether were added, extracted with 2×25 ml water, 3×20 ml 1N NaOH, 1×20 ml brine, dried over Mg₂SO₄, and the solvent was removed under vacuum—To remove the triphenylphosphineoxide, the residue was stirred 30 min in n-Heptane/diethylether, solids were filtered away and the solvent was removed under vacuum. Chromatography (50 g SiO2, Heptane/EA 0 to 30% in 40 min yielded 1.21 g of the desired product as a white solid. MS (ESI): m/z=302.2 [M-56+H]⁺.

### Step c) rel-(3R,4R)-4-((2-chloro-4-fluorophenoxy)methyl)-3-methylpiperidine hydrochloride

Deprotection was achieved in analogy to example 47, step b. MS (ESI): m/z=258.2 [M+H]⁺.

### Example 40

### (4aR,8aS)-6-(3-((4-(Difluoromethoxy)benzyl)oxy)azetidine-1-carbonyl)hexahydro-2H-pyrido[4,3-b][1,4]oxazin-3(4H)-one

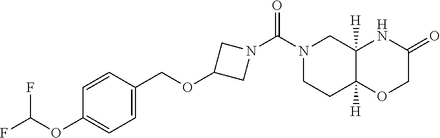 [SMILES: O=C1CO[C@H]2CCN(C(=O)N3CC(OCc4ccc(OC(F)F)cc4)C3)C[C@H]2N1]

Example 40 was prepared in analogy as described for example 16, starting from 4-nitrophenyl (4aR,8aS)-3-oxohexahydro-2H-pyrido[4,3-b][1,4]oxazine-6(5H)-carboxylate (BB2a) and 3-((4-(difluoromethoxy)benzyl)oxy)azetidine trifluoroacetate. Purification by preparative HPLC: YMC-Triart C18, 12 nm, 5 μm, 100×30 mm, 11 min run time, gradient 15-35-50-100 ACN in water+0.1% HCOOH. MS (ESI): m/z=412.3 [M+H]⁺.

### Step a) 3-((4-(difluoromethoxy)benzyl)oxy)azetidine trifluoroacetate

Prepared as described for Example 31, steps a and b. MS (ESI): m/z=230.2 [M+H]⁺.

### Example 41

### (4aR,8aS)-6-(3-((2-Chloro-4-(trifluoromethyl)benzyl)oxy)cyclobutane-1-carbonyl)hexahydro-2H-pyrido[4,3-b][1,4]oxazin-3(4H)-one

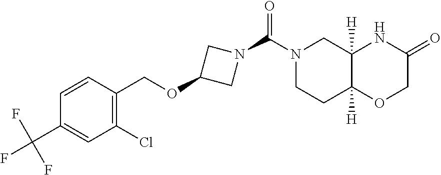

### and Example 42

### (4aR,8aS)-6-(3-((2-Chloro-4-(trifluoromethyl)benzyl)oxy)cyclobutane-1-carbonyl)hexahydro-2H-pyrido[4,3-b][1,4]oxazin-3(4H)-one

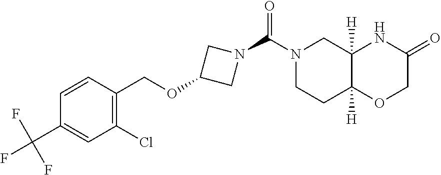

To a solution of 3-((2-chloro-4-(trifluoromethyl)benzyl)oxy)cyclobutane-1-carboxylic acid (270 mg, 875 μmol) in DMF (4.37 mL) were added HATU (366 mg, 962 μmol) and DIPEA (339 mg, 458 μL, 2.62 mmol) and the mixture was stirred at RT for 15 min before (4aR,8aS)-hexahydro-2H-pyrido[4,3-b][1,4]oxazin-3(4H)-one (BB1a, 137 mg, 875 μmol) was added and the mixture was stirred for 2 h at RT. The reaction mixture was purified by reverse-phase HPLC to give a crude product containing both stereoisomers (cis and trans) as an amorphous white solid (201 mg). The mixture was purified by chiral SFC to obtain the two title compounds:

Example 41, 163 mg (41%), cis isomer, yellow oil. MS (ESI): m/z=447.3 [M+H]⁺

Example 42, 54 mg (14%), trans isomer, off-white solid. MS (ESI): m/z=447.3 [M+H]⁺

### Step a) methyl 3-((2-chloro-4-(trifluoromethyl)benzyl)oxy)cyclobutane-1-carboxylate

To a solution of methyl 3-hydroxycyclobutane-1-carboxylate (215 mg, 1.66 mmol) in dry THF (4.14 mL) was added potassium tert-butoxide 1.65 M solution in THF (1.05 mL, 1.74 mmol) and the yellow reaction mixture was stirred at RT for 20 min followed by addition of 1-(bromomethyl)-2-chloro-4-(trifluoromethyl)benzene (453 mg, 1.66 mmol) in one portion. The crude reaction was diluted with ethyl acetate and extracted with aq. sat. NaHCO₃, the organic phase was collected and the aqueous phase was back-extracted with ethyl acetate. The combined organic layers were dried over sodium sulfate, filtered and concentrated. The crude was immobilized on Isolute and purified by column chromatography (0 to 30% EtOAc in heptanes) to afford methyl 3-((2-chloro-4-(trifluoromethyl)benzyl)oxy)cyclobutane-1-carboxylate (275 mg, 810 μmol, 48.9% yield) as a colorless oil containing a mixture of isomers which was carried on to the next step. MS (ESI): m/z=309.2 [M+H]⁺

### Step b) 3-((2-chloro-4-(trifluoromethyl)benzyl)oxy)cyclobutane-1-carboxylic Acid

To a solution of methyl 3-((2-chloro-4-(trifluoromethyl)benzyl)oxy)cyclobutane-1-carboxylate (275 mg, 852 μmol) in THF (2.84 mL) was added LiOH 4.0 M aqueous solution (639 μL, 2.56 mmol) and the reaction mixture was stirred at RT for 23 h. The reaction mixture was partitioned between ethyl acetate and aq. HCl 1N solution, the organic phase was collected and the aqueous phase was back-extracted with ethyl acetate twice. The combined organic phases were dried over sodium sulfate and evaporated down to dryness to yield 3-((2-chloro-4-(trifluoromethyl)benzyl)oxy)cyclobutane-1-carboxylic acid (270 mg, 832 μmol, 97.6% yield) as an off-white oil. The crude product was used without further purification. MS (ESI): m/z=323.1 [M+H]⁺

### Example 43

### (4aR,8aS)-6-(3-((5-(Trifluoromethyl)pyridin-2-yl)methoxy)azetidine-1-carbonyl)hexahydro-2H-pyrido[4,3-b][1,4]oxazin-3(4H)-one

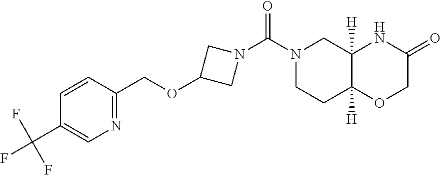 [SMILES: O=C1CO[C@H]2CCN(C(=O)N3CC(OCc4ccc(C(F)(F)F)cn4)C3)C[C@H]2N1]

Example 43 was prepared in analogy as described for example 16, starting from 4-nitrophenyl (4aR,8aS)-3-oxohexahydro-2H-pyrido[4,3-b][1,4]oxazine-6(5H)-carboxylate (BB2a) and 2-((azetidin-3-yloxy)methyl)-5-(trifluoromethyl)pyridine bis(2,2,2-trifluoroacetate). Purification by preparative HPLC: YMC-Triart C18, 12 nm, 5 μm, 100×30 mm, 11 min run time, gradient 15-35-50-100 ACN in water+0.1% HCOOH. MS (ESI): m/z=415.3 [M+H]⁺.

### Step a) 2-((azetidin-3-yloxy)methyl)-5-(trifluoromethyl)pyridine bis(2,2,2-trifluoroacetate)

Prepared as described for Example 31, steps a and b. MS (ESI): m/z=233.2 [M+H]⁺.

### Example 44

### (4aR,8aS)-6-(3-((4-(Trifluoromethoxy)benzyl)oxy)azetidine-1-carbonyl)hexahydro-2H-pyrido[4,3-b][1,4]oxazin-3(4H)-one

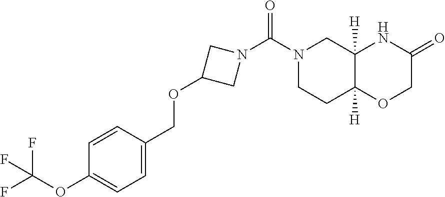 [SMILES: O=C1CO[C@H]2CCN(C(=O)N3CC(OCc4ccc(OC(F)(F)F)cc4)C3)C[C@H]2N1]

Example 44 was prepared in analogy as described for example 16, starting from 4-nitrophenyl (4aR,8aS)-3-oxohexahydro-2H-pyrido[4,3-b][1,4]oxazine-6(5H)-carboxylate (BB2a) and 3-((4-(trifluoromethoxy)benzyl)oxy)azetidine trifluoroacetate. Purification by preparative HPLC: YMC-Triart C18, 12 nm, 5 μm, 100×30 mm, 11 min run time, gradient 25-45-60-100 ACN in water+0.1% HCOOH. MS (ESI): m/z=430.2 [M+H]⁺.

### Step a) 3-((4-(trifluoromethoxy)benzyl)oxy)azetidine trifluoroacetate

Prepared as described for Example 31, steps a and b. MS (ESI): m/z=248.1 [M+H]⁺.

### Example 45

### N-(2-chloro-4-fluorophenyl)-1-((4aR,8aS)-3-oxooctahydro-2H-pyrido[4,3-b][1,4]oxazine-6-carbonyl)azetidine-3-carboxamide

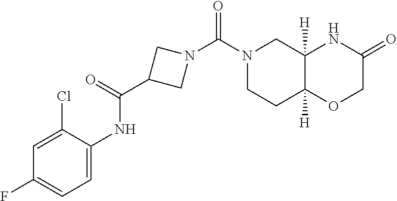 [SMILES: O=C1CO[C@H]2CCN(C(=O)N3CC(C(=O)Nc4ccc(F)cc4Cl)C3)C[C@H]2N1]

To a solution of 4-nitrophenyl (4aR,8aS)-3-oxohexahydro-2H-pyrido[4,3-b][1,4]oxazine-6(5H)-carboxylate (BB2a, 40 mg, 124 μmol) in a mixture of CH₃CN (1 mL) was added DIPEA (40.2 mg, 54.4 μL, 311 μmol) and N-(2-chloro-4-fluorophenyl)azetidine-3-carboxamide trifluoroacetate (49.1 mg, 143 μmol). The reaction vial was stirred at 80° C. for 18 h. The crude material was submitted for reversed-phase HPLC purification to yield 41.2 mg of the title compound. MS (ESI): m/z=411.2 [M+H]⁺.

### Step a) tert-butyl 3-[(2-chloro-4-fluoro-phenyl)carbamoyl]azetidine-1-carboxylate

To a solution of 2-chloro-4-fluoroaniline (500 mg, 2.86 mmol), 1-BOC-azetidine-3-carboxylic acid (576 mg, 2.86 mmol) and 4-dimethylaminopyridine (35 mg, 0.290 mmol) in THF (10 mL) was added 1-(3-dimethylaminopropyl)-3-ethylcarbodiimide hydrochloride (714 mg, 3.72 mmol) at 0° C. The mixture was heated to 30° C. and stirred for 16 h. To the mixture was added ethyl acetate (5 mL), washed with brine (10 mL×3) and dried over Na₂SO₄. The organic layer was concentrated in vacuo to obtain crude product (0.8 g) as a yellow oil. The crude product was purified by preparative-HPLC and dried by lyophilization to give the desired product tert-butyl 3-[(2-chloro-4-fluoro-phenyl)carbamoyl]azetidine-1-carboxylate (672 mg, 71% yield) as a white solid.

### Step b) N-(2-chloro-4-fluoro-phenyl)azetidine-3-carboxamide; 2,2,2-trifluoroacetic acid

To a solution of tert-butyl 3-[(2-chloro-4-fluoro-phenyl)carbamoyl]azetidine-1-carboxylate (350 mg, 1.06 mmol) in DCM (3.5 mL) was added trifluoroacetic acid (0.7 mL, 9.09 mmol) at 0° C. The solution was stirred at 0° C. for 2 h. The reaction was concentrated in vacuo to give crude product (400 mg) as a light yellow oil. The crude product was purified by prep-HPLC (TFA) and concentrated in vacuo, then dried by lyophilization to obtain the title compound (331 mg, 0.960 mmol, 91% yield) as a white solid. MS (ESI): m/z=229.1 [M+H]⁺.

### Example 46

### (4aS,8aR)-6-(3-(1-(2-Chloro-4-(trifluoromethyl)phenoxy)ethyl)azetidine-1-carbonyl)hexahydro-2H-pyrido[4,3-b][1,4]oxazin-3(4H)-one

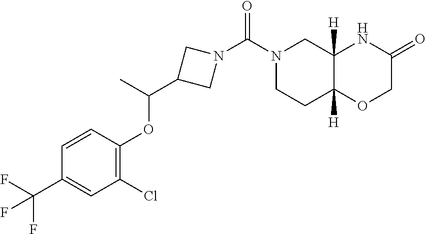 [SMILES: CC(Oc1ccc(C(F)(F)F)cc1Cl)C1CN(C(=O)N2CC[C@H]3OCC(=O)N[C@H]3C2)C1]

Example 46 was prepared in analogy as described for example 16, starting from 4-nitrophenyl (4aS,8aR)-3-oxohexahydro-2H-pyrido[4,3-b][1,4]oxazine-6(5H)-carboxylate (BB2b) and 3-(1-(2-chloro-4-(trifluoromethyl)phenoxy)ethyl)azetidine trifluoroacetate. Purification by preparative HPLC: YMC-Triart C18, 12 nm, 5 μm, 100×30 mm, 11 min run time, gradient 25-45-60-100 ACN in water+0.1% TEA. MS (ESI): m/z=462.2 [M+H]⁺.

### Step a) 3-(1-(2-chloro-4-(trifluoromethyl)phenoxy)ethyl)azetidine trifluoroacetate

Prepared as described for Example 31, steps a and b. MS (ESI): m/z=280.2 [M+H]⁺.

### Example 47

### (4aR,8aS)-6-(4-(3-(Trifluoromethyl)pyridazin-4-yl)piperidine-1-carbonyl)hexahydro-2H-pyrido[4,3-b][1,4]oxazin-3(4H)-one

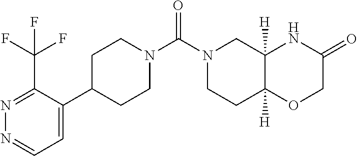 [SMILES: O=C1CO[C@H]2CCN(C(=O)N3CCC(c4ccnnc4C(F)(F)F)CC3)C[C@H]2N1]

To an ice-cold solution of bis(trichloromethyl) carbonate (39.9 mg, 134 μmol) in DCM (2 ml) were added sodium bicarbonate (64.5 mg, 768 μmol) and (4aR,8aS)-hexahydro-2H-pyrido[4,3-b][1,4]oxazin-3(4H)-one (BB1a, 30 mg, 192 μmol) and the mixture was stirred overnight at RT. To the suspension was added 4-(piperidin-4-yl)-3-(trifluoromethyl)pyridazine hydrochloride (51.4 mg, 192 μmol) and DIPEA (99.3 mg, 134 μl, 768 μmol). The suspension was stirred at RT for 3 hours. The reaction mixture was poured on water and DCM and the layers were separated. The aqueous layer was extracted three times with DCM (slow separation). The organic layers were washed twice with water, dried over MgSO₄, filtered, treated with silica gel and evaporated. The compound was purified by silica gel chromatography on a 10 g column using an MPLC system eluting with a gradient of DCM: DCM+10% MeOH (0 to 100 in 20 min) and subsequent preparative HPLC (Gemini NX, 12 nm, 5 μm, 100×30 mm, 15 min gradient 10-25-40-100% ACN in water+0.1% HCOOH), to get the desired compound as a white solid. (13.5 mg, 17%). MS (ESI): m/z=414.3 [M+H]⁺.

### Step a) tert-butyl 4-(3-(trifluoromethyl)pyridazin-4-yl)piperidine-1-carboxylate

Potassium (1-(tert-butoxycarbonyl)piperidin-4-yl)trifluoroborate (649 mg, 2.23 mmol), silver nitrate (68.8 mg, 405 μmol, Eq: 0.2) and potassium persulfate (2.74 g, 10.1 mmol) were weighed in a reaction tube equipped with a stir bar. 1,2-Dichloroethane (2 ml), Water (2 ml), 3-(trifluoromethyl)pyridazine (300 mg, 2.03 mmol) and TFA (462 mg, 312 μl, 4.05 mmol) were successively added, and the tube was sealed. The reaction was vigorously stirred at RT for 24 h. Then the reaction mixture was poured into 20 ml of a 1/1 v/v mixture of sat. aq. NaHCO3 and 5% aq. NaS2O3 and the resulting solution was extracted three times with DCM, the combined org. layers were dried (MgSO₄) and evaporated to afford the crude product. Purification by flash chromatography (heptane/EA 0 to 80% in 35 min) afforded the desired product as a yellow viscouos oil (180 mg, 80%). MS (ESI): m/z=332.2 [M+H]⁺.

### Step b) 4-(piperidin-4-yl)-3-(trifluoromethyl)pyridazine hydrochloride

tert-butyl 4-(3-(trifluoromethyl)pyridazin-4-yl)piperidine-1-carboxylate (180 mg, 543 μmol) was dissolved in DCM (1 ml), HCl 2M in Ether (2.72 ml, 5.43 mmol) was added. The reaction mixture was stirred for 6 hours and then concentrated on high vacuum, yielding 165 mg of the desired product as a yellow solid. Directly used for next step without purification. MS (ESI): m/z=232.2 [M+H]⁺.

### Example 48

### (4aR,8aS)-6-[3-[5-(2,4-Difluorophenyl)-4H-1,2,4-triazol-3-yl]azetidine-1-carbonyl]-4,4a,5,7,8,8a-hexahydropyrido[4,3-b][1,4]oxazin-3-one

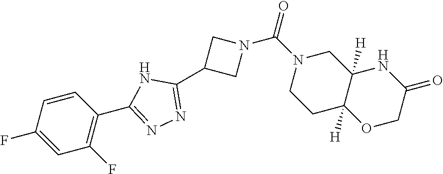 [SMILES: O=C1CO[C@H]2CCN(C(=O)N3CC(c4nnc(-c5ccc(F)cc5F)[nH]4)C3)C[C@H]2N1]

To a solution of (4aR,8aS)-6-(1H-1,2,4-triazole-1-carbonyl)hexahydro-2H-pyrido[4,3-b][1,4]oxazin-3(4H)-one (90 mg, 358 μmol, BB3) in dry DMF (1.5 ml) was added DIPEA (375 μl, 2.15 mmol) and 3-(azetidin-3-yl)-5-(2,4-difluorophenyl)-4H-1,2,4-triazole bis(2,2,2-trifluoroacetate) (183 mg, 394 μmol) after which the reaction mixture was stirred at 80° C. for 18 hours. The crude reaction solution was purified on a preparative HPLC coumn (Gemini NX, 12 nm, 5 μm, 100×30 mm, 15 min gradient 10-25-40-100% ACN in water+0.1% HCOOH) to get the desired compound. MS (ESI): m/z=419.1 [M+H]⁺.

### Step a) 3-(azetidin-3-yl)-5-(2,4-difluorophenyl)-4H-1,2,4-triazole bis(2,2,2-trifluoroacetate)

To a solution of tert-butyl 3-(5-(2,4-difluorophenyl)-4H-1,2,4-triazol-3-yl)azetidine-1-carboxylate (570 mg, 1.69 mmol) in CH2Cl2 (3 ml) was added TFA (653 μl, 8.47 mmol) and the reaction mixture was stirred at r.t for 18 hours. Volatiles were removed in vacuo to yield the desired product (845 mg, 96.6%). MS (ESI): m/z=237.1 [M+H]⁺. It was used without further purification for the next step.

### Step b) tert-butyl 3-(5-(2,4-difluorophenyl)-4H-1,2,4-triazol-3-yl)azetidine-1-carboxylate

To a solution of tert-butyl 3-cyanoazetidine-1-carboxylate (450 mg, 2.47 mmol, CAS RN 142253-54-1) in 1-butanol (9 ml) was added 2,4-difluorobenzohydrazide (425 mg, 2.47 mmol, CAS RN 118737-62-5) and potassium carbonate (341 mg, 2.47 mmol) after which the reaction was stirred at 110° C. for 18 hours. Volatiles were removed in vacuo, the crude residue was suspended in ethyl acetate and extracted with aq. HCl 0.5M. The organic phase was collected and the aqueous phase was back-extracted with ethyl acetate. The combined organic phases were dried over sodium sulfate and evaporated down to dryness.

The crude material was purified by flash chromatography with a 80 gr SiO₂ column, eluent mixture of dichloromethane and methanol (0% to 10%) to get the desired product (575 mg, 65.8%). MS (ESI): m/z=335.3 [M+H]⁻.

### Example 49

### (4aR,8aS)-6-[3-[[4-Fluoro-2-(trifluoromethyl)phenyl]methoxy]azetidine-1-carbonyl]-4,4a,5,7,8,8a-hexahydropyrido[4,3-b][1,4]oxazin-3-one

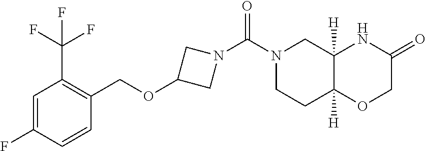 [SMILES: O=C1CO[C@H]2CCN(C(=O)N3CC(OCc4ccc(F)cc4C(F)(F)F)C3)C[C@H]2N1]

To a yellow suspension of 3-((4-fluoro-2-(trifluoromethyl)benzyl)oxy)azetidine 4-methylbenzenesulfonate (164 mg, 249 μmol) and DIPEA (152 μl, 871 μmol) in Acetonitrile (1 ml) was added 4-nitrophenyl (4aR,8aS)-3-oxohexahydro-2H-pyrido[4,3-b][1,4]oxazine-6(5H)-carboxylate (80 mg, 249 μmol, BB2a) and the slowly formed yellow solution was stirred at RT overnight. The suspension was evaporated. The product was purified on a preparative HPLC (YMC-Triart column) using a gradient of acetonitrile 20-40-55-100% in water (containing 0.1% TEA) to get the desired compound as a colorless solid (0.100 g, 93.1%). MS (ESI): m/z=432.2 [M+H]⁺.

### Step a) 3-((4-fluoro-2-(trifluoromethyl)benzyl)oxy)azetidine 4-methylbenzenesulfonate

To a solution of tert-butyl 3-((4-fluoro-2-(trifluoromethyl)benzyl)oxy)azetidine-1-carboxylate (200 mg, 573 μmol) in EtOAc (2 ml) was added 4-methylbenzenesulfonic acid monohydrate (131 mg, 687 μmol) and the mixture was heated at reflux over night. The solution was evaporated to get the desired product as a colorless solid (0.377 g; 100%). MS (ESI): m/z=250.2 [M+H]⁺.

### Step b) tert-butyl 3-((4-fluoro-2-(trifluoromethyl)benzyl)oxy)azetidine-1-carboxylate

To a solution of tert-butyl 3-hydroxyazetidine-1-carboxylate (1 g, 5.77 mmol, CAS RN 141699-55-0) in dry THF (25 ml) under Ar atmosphere, potassium tert-butoxide 1.65M solution in THF (3.85 ml, 6.35 mmol) was added. Then the reaction was covered in aluminum foil to prevent light impact and the mixture was stirred at RT for 30 min. After that, the addition of 1-(bromomethyl)-4-fluoro-2-(trifluoromethyl)benzene (1.48 g, 5.77 mmol, CAS RN 206860-48-2) took place. Then, the reaction mixture was stirred for 17 h at RT. The reaction mixture was diluted with EtOAc and extracted with 1M aqueous NaHCO₃ solution. The organic phase was collected and the aqueous phase underwent back-extraction with EtOAc. The combined organic layers were dried over Na₂SO₄ and, consequently, evaporated to dryness to give a yellow viscous liquid (2.05 g, 99.6%). MS (ESI): m/z=294.2 [M-C4H8+H]⁺.

### Synthesis of Building Blocks

### BB1a & BB1b

### (+)-cis-4a,5,6,7,8,8a-Hexahydro-4H-pyrido[4,3-b][1,4]oxazin-3-one

### and

### (−)-cis-4a,5,6,7,8,8a-Hexahydro-4H-pyrido[4,3-b][1,4]oxazin-3-one

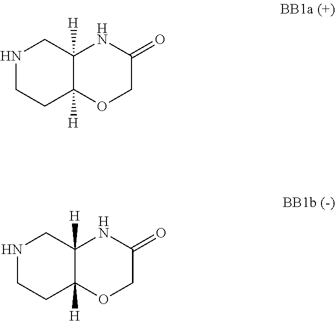 [SMILES: O=C1CO[C@H]2CCNC[C@H]2N1 ; O=C1CO[C@@H]2CCNC[C@@H]2N1]

The enantiomers of rac-(4aR,8aS)-hexahydro-2H-pyrido[4,3-b][1,4]oxazin-3(4H)-one dihydrochloride (BB1, 500 mg, 2.18 mmol, ChemBridge Corporation) were separated by preparative chiral HPLC (ReprosilChiral NR column) using an isocratic mixture of EtOH (containing 0.05% of NH₄OAc): n-heptane (30:70).

First eluting enantiomer: (+)-cis-4a,5,6,7,8,8a-Hexahydro-4H-pyrido[4,3-b][1,4]oxazin-3-one (BB1a). Yellow solid (0.150 g; 44.0%). MS (ESI): m/z=157.1 [M+H]⁺.

Second eluting enantiomer: (−)-cis-4a,5,6,7,8,8a-Hexahydro-4H-pyrido[4,3-b][1,4]oxazin-3-one. (BB1b). Yellow solid (0.152 g; 44.6%). MS (ESI): m/z=157.1 [M+H]⁺.

### BB2a and BB2b

### 4-Nitrophenyl (4aR,8aS)-3-oxohexahydro-2H-pyrido[4,3-b][1,4]oxazine-6(5H)-carboxylate (BB2a)

### and

### 4-nitrophenyl (4aS,8aR)-3-oxohexahydro-2H-pyrido[4,3-b][1,4]oxazine-6(5H)-carboxylate (BB2b)

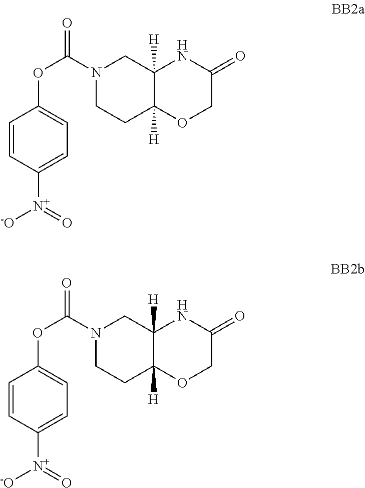

To a suspension of rac-(4aR,8aS)-hexahydro-2H-pyrido[4,3-b][1,4]oxazin-3(4H)-one; dihydrochloride salt (4.5 g, 19.6 mmol, BB1) in dry DCM (125 mL) at 0° C. was added DIPEA (6.35 g, 8.58 mL, 49.1 mmol) followed by 4-nitrophenyl carbonochloridate (4.35 g, 21.6 mmol). The reaction mixture was stirred at 0° C. for 10 min and at RT for 2 hours. The crude reaction was diluted with DCM and transferred into a separating funnel for extraction with sat. aq. Na₂CO₃ solution. The organic phase was collected and the aqueous phase was back-extracted with DCM. The combined organic phases were dried over Na₂SO₄ and evaporated down to dryness to yield 6.62 g of a crude racemic product (BB7) as a yellow solid. The crude material was directly submitted for a chiral SFC separation to yield enantiomer BB2b (2.72 g, second eluting enantiomer) as a yellow solid and enantiomer BB2a (3.25 g, first eluting enantiomer) as a light beige solid but contaminated with BB2b. A further SFC chiral separation was carried out to yield 2.71 g of BB2a. MS (ESI): m/z=322.2 [M+H]⁺ for both enantiomers.

BB3

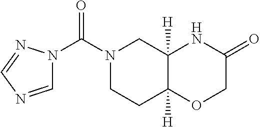 [SMILES: O=C1CO[C@H]2CCN(C(=O)n3cncn3)C[C@H]2N1]

To a suspension of (4aR,8aS)-4a,5,6,7,8,8a-hexahydro-4H-pyrido[4,3-b][1,4]oxazin-3-one (100 mg, 0.640 mmol, BB1a) in acetonitrile (2 mL) were added bis(1,2,4-triazol-1-yl)methanone (110.34 mg, 0.672 mmol, CAS RN 41864-22-6) and DIEA (117.42 uL, 0.672 mmol) and the solution was stirred overnight at RT. The solution was evaporated. The residue was taken up in DCM and 1M aqueous Na₂CO₃ solution and the layers were separated. The aqueous layer was extracted once with DCM. The organic layers were dried over MgSO4, filtered and evaporated to get the desired product as a colorless amorphous (0.090, 54.2%). MS (ESI): m/z=252.2 [M+H]⁺.

### Example 50

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 51

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1-41. (canceled)

42. A compound of formula (I): 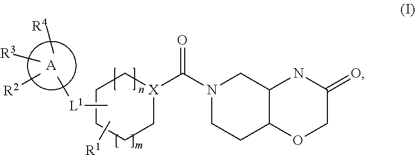
or a pharmaceutically acceptable salt thereof, wherein:
R¹ is hydrogen or C₁-C₆-alkyl;
R², R³, and R⁴ are independently selected from the group consisting of hydrogen, a group 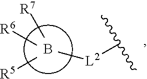
C₁-C₆-alkyl, C₁-C₆-alkoxy, halo-C₁-C₆-alkyl, halo-C₁-C₆-alkoxy, halogen, and SF₅;
R⁵, R⁶, and R⁷ are independently selected from the group consisting of hydrogen, halogen, and halo-C₁-C₆-alkyl;
X is CH or N;
m and n are both 1; or
m and n are both 0;
A is C₆-C₁₄-aryl or 5-14 membered heteroaryl;
L¹ is a covalent bond, —CH₂—, —OCHR^{L}—, —CHR^{L}O—, or —NHC(O)—;
R^{L} is hydrogen or C₁-C₆-alkyl; and
B is
(i) C₆-C₁₄-aryl and L² is a covalent bond; or
(ii) 3-14 membered heterocyclyl or C₃-C₁₀-cycloalkyl; and L² is a covalent bond, —O—, or —CH₂O—;
wherein the compound is not:
rac-cis-6-(4-(5-Chloro-1-methyl-1H-indol-3-yl)piperidine-1-carbonyl)hexahydro-2H-pyrido[4,3-b][1,4]oxazin-3(4H)-one;
rac-cis-6-(4-(9H-Fluoren-9-yl)piperidine-1-carbonyl)hexahydro-2H-pyrido[4,3-b][1,4]oxazin-3(4H)-one;
(+)-cis-6-[4-(6-Fluoro-1H-indol-3-yl)piperidine-1-carbonyl]-4,4a,5,7,8,8a-hexahydropyrido[4,3-b][1,4]oxazin-3-one;
rac-cis-6-[4-(6-Fluoro-1H-indol-3-yl)piperidine-1-carbonyl]-4,4a,5,7,8,8a-hexahydropyrido[4,3-b][1,4]oxazin-3-one;
rac-cis-6-(4-(5-Fluorobenzo[d]isoxazol-3-yl)piperidine-1-carbonyl)hexahydro-2H-pyrido[4,3-b][1,4]oxazin-3(4H)-one;
rac-cis-6-(4-(5-Chloro-1H-indol-3-yl)piperazine-1-carbonyl)hexahydro-2H-pyrido[4,3-b][1,4]oxazin-3(4H)-one;
rac-cis-6-(4-(1-Methyl-1H-indazol-5-yl)piperidine-1-carbonyl)hexahydro-2H-pyrido[4,3-b][1,4]oxazin-3(4H)-one;
(+)- or (−)-cis-6-(4-(5-Chloro-1-cyclopropyl-1H-indol-3-yl)piperidine-1-carbonyl)hexahydro-2H-pyrido[4,3-b][1,4]oxazin-3(4H)-one;
(+)- or (−)-cis-6-(4-(5-Chloro-1-(oxetan-3-yl)-1H-indol-3-yl)piperidine-1-carbonyl)hexahydro-2H-pyrido[4,3-b][1,4]oxazin-3(4H)-one;
rac-cis-6-(4-(1-(2-Chloro-4-fluorophenoxy)ethyl)piperidine-1-carbonyl)hexahydro-2H-pyrido[4,3-b][1,4]oxazin-3(4H)-one;
rac-cis-6-(4-(5-(Trifluoromethyl)pyridin-3-yl)piperidine-1-carbonyl)hexahydro-2H-pyrido[4,3-b][1,4]oxazin-3(4H)-one;
(+) or (−)-cis-6-(4-((S or R)-1-(2-chloro-4-fluorophenoxy)ethyl)piperidine-1-carbonyl)hexahydro-2H-pyrido[4,3-b][1,4]oxazin-3(4H)-one;
(+) or (−)-cis-6-(4-((R or S)-1-(2-chloro-4-fluorophenoxy)ethyl)piperidine-1-carbonyl)hexahydro-2H-pyrido[4,3-b][1,4]oxazin-3(4H)-one;
(+) or (−)-cis-6-(4-(5-Methoxypyridin-3-yl)piperidine-1-carbonyl)hexahydro-2H-pyrido[4,3-b][1,4]oxazin-3(4H)-one;
(+) or (−)-cis-6-(4-(5-(Trifluoromethoxy)pyridin-2-yl)piperidine-1-carbonyl)hexahydro-2H-pyrido[4,3-b][1,4]oxazin-3(4H)-one;
(+) or (−)-cis-6-(4-(5-Ethylpyridin-3-yl)piperidine-1-carbonyl)hexahydro-2H-pyrido[4,3-b][1,4]oxazin-3(4H)-one;
(+) or (−)-cis-6-(4-(5-(1,1-Difluoroethyl)pyridin-2-yl)piperidine-1-carbonyl)hexahydro-2H-pyrido[4,3-b][1,4]oxazin-3(4H)-one;
(+) or (−)-cis-6-(4-(6-Chloro-1-methyl-H-indazol-3-yl)piperidine-1-carbonyl)hexahydro-2H-pyrido[4,3-b][1,4]oxazin-3(4H)-one;
(4aR,8aS)-6-((3R)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-((3 S)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-((4S)-4-(5-(1,1-Difluoroethyl)pyridin-2-yl)-3-methylpiperidine-1-carbonyl)hexahydro-2H-pyrido[4,3-b][1,4]oxazin-3(4H)-one;
(4aR,8aS)-6-(4-(2-Cyclopropylpyridin-4-yl)piperidine-1-carbonyl)hexahydro-2H-pyrido[4,3-b][1,4]oxazin-3(4H)-one;
(4aR,8aS)-6-(3-(1-(2-Chloro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-(1-(2-Chloro-4-(trifluoromethyl)phenoxy)ethyl)azetidine-1-carbonyl)hexahydro-2H-pyrido[4,3-b][1,4]oxazin-3(4H)-one;
(+) or (−)-(4aR,8aS)-6-[3-[1-[4-(Trifluoromethyl)phenyl]ethoxy]azetidine-1-carbonyl]-4,4a,5,7,8,8a-hexahydropyrido[4,3-b][1,4]oxazin-3-one;
(4aR,8aS)-6-(3-(1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-(1-(4-(Trifluoromethyl)phenoxy)ethyl)azetidine-1-carbonyl)hexahydro-2H-pyrido[4,3-b][1,4]oxazin-3(4H)-one;
(4aR,8aS)-6-[4-[1-[4-(Trifluoromethyl)phenyl]ethoxy]piperidine-1-carbonyl]-4,4a,5,7,8,8a-hexahydropyrido[4,3-b][1,4]oxazin-3-one;
(4aR,8aS)-6-(3-((S)-1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-((R)-1-(2-Fluoro-4-(trifluoromethyl)phenoxy)ethyl)azetidine-1-carbonyl)hexahydro-2H-pyrido[4,3-b][1,4]oxazin-3(4H)-one;
(4aR,8aS)-6-(3-((S)-1-(4-(Trifluoromethyl)phenoxy)ethyl)azetidine-1-carbonyl)hexahydro-2H-pyrido[4,3-b][1,4]oxazin-3(4H)-one;
(4aR,8aS)-6-(3-((R)-1-(4-(trifluoromethyl)phenoxy)ethyl)azetidine-1-carbonyl)hexahydro-2H-pyrido[4,3-b][1,4]oxazin-3(4H)-one;
(4aR,8aS)-6-(4-(5-Methyl-6-(trifluoromethyl)pyridin-3-yl)piperidine-1-carbonyl)hexahydro-2H-pyrido[4,3-b][1,4]oxazin-3(4H)-one;
(4aR,8aS)-6-(4-(5,6,7,8-Tetrahydroquinolin-4-yl)piperidine-1-carbonyl)hexahydro-2H-pyrido[4,3-b][1,4]oxazin-3(4H)-one;
(4aR,8aS)-6-(3-(4-Bromophenyl)azetidine-1-carbonyl)hexahydro-2H-pyrido[4,3-b][1,4]oxazin-3(4H)-one;
(4aR,8aS)-6-(3-(4′-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(2′-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(2′,4′-Dichloro-[1,1′-biphenyl]-4-yl)azetidine-1-carbonyl)hexahydro-2H-pyrido[4,3-b][1,4]oxazin-3(4H)-one;
(4aR,8aS)-6-(3-(1-(4-(Trifluoromethyl)phenyl)ethoxy)azetidine-1-carbonyl)hexahydro-2H-pyrido[4,3-b][1,4]oxazin-3(4H)-one;
(4aR,8aS)-6-(3-(1-(4-(Trifluoromethyl)phenyl)ethoxy)azetidine-1-carbonyl)hexahydro-2H-pyrido[4,3-b][1,4]oxazin-3(4H)-one;
(4aR,8aS)-6-[3-(S or R)-[1-(2-Chloro-4-fluoro-phenyl)ethoxy]azetidine-1-carbonyl]-4,4a,5,7,8,8a-hexahydropyrido[4,3-b][1,4]oxazin-3-one;
(4aR,8aS)-6-[3-(R or S)-[1-(2-Chloro-4-fluoro-phenyl)ethoxy]azetidine-1-carbonyl]-4,4a,5,7,8,8a-hexahydropyrido[4,3-b][1,4]oxazin-3-one;
(4aR,8aS)-6-[3-[4-(Trifluoromethoxy)phenyl]azetidine-1-carbonyl]-4,4a,5,7,8,8a-hexahydropyrido[4,3-b][1,4]oxazin-3-one;
(4aR,8aS)-6-(3-(4-Bromophenyl)-3-fluoroazetidine-1-carbonyl)hexahydro-2H-pyrido[4,3-b][1,4]oxazin-3(4H)-one;
(4aR,8aS)-6-(3-(4-Bromophenyl)-3-hydroxyazetidine-1-carbonyl)hexahydro-2H-pyrido[4,3-b][1,4]oxazin-3(4H)-one;
(4aR,8aS)-6-(3-(4-Bromophenyl)-3-methylazetidine-1-carbonyl)hexahydro-2H-pyrido[4,3-b][1,4]oxazin-3(4H)-one;
(4aR,8aS)-6-(3-(2′-(Trifluoromethyl)-[1,1′-biphenyl]-4-yl)azetidine-1-carbonyl)hexahydro-2H-pyrido[4,3-b][1,4]oxazin-3(4H)-one;
(4aR,8aS)-6-(3-(2′,4′-Difluoro-[1,1′-biphenyl]-4-yl)azetidine-1-carbonyl)hexahydro-2H-pyrido[4,3-b][1,4]oxazin-3(4H)-one;
(4aR,8aS)-6-(3-(4-(3-(Trifluoromethyl)azetidin-1-yl)phenyl)azetidine-1-carbonyl)hexahydro-2H-pyrido[4,3-b][1,4]oxazin-3(4H)-one;
(4aR,8aS)-6-(3-(2-Chloro-[1,1′-biphenyl]-4-yl)azetidine-1-carbonyl)hexahydro-2H-pyrido[4,3-b][1,4]oxazin-3(4H)-one;
(4aR,8aS)-6-(3-(4-Bromo-3-chlorophenyl)azetidine-1-carbonyl)hexahydro-2H-pyrido[4,3-b][1,4]oxazin-3(4H)-one;
(4aR,8aS)-6-[3-[4-(4-Chloro-2-fluoro-phenyl)phenyl]azetidine-1-carbonyl]-4,4a,5,7,8,8a-hexahydropyrido[4,3-b][1,4]oxazin-3-one;
(4aR,8aS)-6-[3-[4-(2-Chloro-4-fluoro-phenyl)phenyl]azetidine-1-carbonyl]-4,4a,5,7,8,8a-hexahydropyrido[4,3-b][1,4]oxazin-3-one;
(4aR,8aS)-6-(3-(3-Bromophenyl)azetidine-1-carbonyl)hexahydro-2H-pyrido[4,3-b][1,4]oxazin-3(4H)-one;
(4aR,8aS)-6-(3-(4-(tert-Butyl)phenyl)azetidine-1-carbonyl)hexahydro-2H-pyrido[4,3-b][1,4]oxazin-3(4H)-one;
(4aR,8aS)-6-[3-(4-Phenylphenyl)azetidine-1-carbonyl]-4,4a,5,7,8,8a-hexahydropyrido[4,3-b][1,4]oxazin-3-one;
(4aR,8aS)-6-[3-[4-[2-(Difluoromethyl)phenyl]phenyl]azetidine-1-carbonyl]-4,4a,5,7,8,8a-hexahydropyrido[4,3-b][1,4]oxazin-3-one;
(4aR,8aS)-6-(3-(6-Chloropyridin-3-yl)azetidine-1-carbonyl)hexahydro-2H-pyrido[4,3-b][1,4]oxazin-3(4H)-one;
(4aR,8aS)-6-(3-(4-(Trifluoromethyl)phenyl)azetidine-1-carbonyl)hexahydro-2H-pyrido[4,3-b][1,4]oxazin-3(4H)-one;
(4aR,8aS)-6-(3-(4-(1,1-Difluoroethyl)phenyl)azetidine-1-carbonyl)hexahydro-2H-pyrido[4,3-b][1,4]oxazin-3(4H)-one;
(4aR,8aS)-6-(3-(6-(2,4-Dichlorophenyl)pyridin-3-yl)azetidine-1-carbonyl)hexahydro-2H-pyrido[4,3-b][1,4]oxazin-3(4H)-one;
(4aR,8aS)-6-(3-(4-(3,3-Difluoroazetidin-1-yl)phenyl)azetidine-1-carbonyl)hexahydro-2H-pyrido[4,3-b][1,4]oxazin-3(4H)-one;
(4aR,8aS)-6-(4-(4-Bromophenyl)piperidine-1-carbonyl)hexahydro-2H-pyrido[4,3-b][1,4]oxazin-3(4H)-one;
(4aR,8aS)-6-[3-[4-(2,2,2-Trifluoroethoxy)phenyl]azetidine-1-carbonyl]-4,4a,5,7,8,8a-hexahydropyrido[4,3-b][1,4]oxazin-3-one;
(4aR,8aS)-6-(3-(4-(2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-(3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(3-Bromophenyl)pyrrolidine-1-carbonyl)hexahydro-2H-pyrido[4,3-b][1,4]oxazin-3(4H)-one;
(4aR,8aS)-6-(4-(4-Bromophenyl)piperazine-1-carbonyl)hexahydro-2H-pyrido[4,3-b][1,4]oxazin-3(4H)-one;
(4aR,8aS)-6-(4-(2′,4′-dichloro-[1,1′-biphenyl]-4-yl)piperidine-1-carbonyl)hexahydro-2H-pyrido[4,3-b][1,4]oxazin-3(4H)-one;
(4aR,8aS)-6-(3-(4-(3-azabicyclo[3.1.0]hexan-3-yl)phenyl)azetidine-1-carbonyl)hexahydro-2H-pyrido[4,3-b][1,4]oxazin-3(4H)-one;
(4aR,8aS)-6-(3-(3-(trifluoromethoxy)phenyl)azetidine-1-carbonyl)hexahydro-2H-pyrido[4,3-b][1,4]oxazin-3(4H)-one;
(4aR,8aS)-6-(3-(6-(2-(trifluoromethyl)pyrrolidin-1-yl)pyridin-3-yl)azetidine-1-carbonyl)hexahydro-2H-pyrido[4,3-b][1,4]oxazin-3(4H)-one;
(4aR,8aS)-6-(3-(4-(5-Azaspiro[2.4]heptan-5-yl)phenyl)azetidine-1-carbonyl)hexahydro-2H-pyrido[4,3-b][1,4]oxazin-3(4H)-one;
(4aR,8aS)-6-(3-(4-(Pentafluoro-16-sulfaneyl)phenyl)azetidine-1-carbonyl)hexahydro-2H-pyrido[4,3-b][1,4]oxazin-3(4H)-one;
(4aR,8aS)-6-(3-(5-Chloropyridin-2-yl)azetidine-1-carbonyl)hexahydro-2H-pyrido[4,3-b][1,4]oxazin-3(4H)-one;
(4aR,8aS)-6-(3-(2-Fluoro-4-(trifluoromethoxy)phenyl)azetidine-1-carbonyl)hexahydro-2H-pyrido[4,3-b][1,4]oxazin-3(4H)-one;
(4aR,8aS)-6-[3-(6-Methoxypyridin-3-yl)azetidine-1-carbonyl]-4,4a,5,7,8,8a-hexahydropyrido[4,3-b][1,4]oxazin-3-one;
(4aR,8aS)-6-(3-(4-Bromophenyl)pyrrolidine-1-carbonyl)hexahydro-2H-pyrido[4,3-b][1,4]oxazin-3(4H)-one;
(4aR,8aS)-6-(3-Phenylazetidine-1-carbonyl)hexahydro-2H-pyrido[4,3-b][1,4]oxazin-3(4H)-one;
(4aR,8aS)-6-(4-Phenylpiperidine-1-carbonyl)hexahydro-2H-pyrido[4,3-b][1,4]oxazin-3(4H)-one;
(4aR,8aS)-6-[3-[4-(2,2,2-Trifluoroethyl)phenyl]azetidine-1-carbonyl]-4,4a,5,7,8,8a-hexahydropyrido[4,3-b][1,4]oxazin-3-one;
(4aR,8aS)-6-[3-[4-[1-(Trifluoromethyl)cyclopropyl]phenyl]azetidine-1-carbonyl]-4,4a,5,7,8,8a-hexahydropyrido[4,3-b][1,4]oxazin-3-one;
(4aR,8aS)-6-[3-[4-(6,6-Difluoro-2-azaspiro[3.3]heptan-2-yl)phenyl]azetidine-1-carbonyl]-4,4a,5,7,8,8a-hexahydropyrido[4,3-b][1,4]oxazin-3-one;
(4aR,8aS)-6-(3-(4-(8-Oxa-3-azabicyclo[3.2.1]octan-3-yl)phenyl)azetidine-1-carbonyl)hexahydro-2H-pyrido[4,3-b][1,4]oxazin-3(4H)-one;
(4aR,8aS)-6-(3-(5-(2,4-Dichlorophenyl)pyridin-2-yl)azetidine-1-carbonyl)hexahydro-2H-pyrido[4,3-b][1,4]oxazin-3(4H)-one;
(4aR,8aS)-6-(3-(4-((S)-2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-((R)-2-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-[3-[4-(1-Piperidyl)phenyl]azetidine-1-carbonyl]-4,4a,5,7,8,8a-hexahydropyrido[4,3-b][1,4]oxazin-3-one;
(4aR,8aS)-6-(3-(4-((R or S)-3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-((S or R)-3-(Trifluoromethyl)pyrrolidin-1-yl)phenyl)azetidine-1-carbonyl)hexahydro-2H-pyrido[4,3-b][1,4]oxazin-3(4H)-one;
(4aR,8aS)-6-(3-(4-(3-Fluoroazetidin-1-yl)phenyl)azetidine-1-carbonyl)hexahydro-2H-pyrido[4,3-b][1,4]oxazin-3(4H)-one;
(4aR,8aS)-6-(3-(3-Fluoro-4-(trifluoromethoxy)phenyl)azetidine-1-carbonyl)hexahydro-2H-pyrido[4,3-b][1,4]oxazin-3(4H)-one;
(4aR,8aS)-6-(3-(3-Methyl-4-(trifluoromethoxy)phenyl)azetidine-1-carbonyl)hexahydro-2H-pyrido[4,3-b][1,4]oxazin-3(4H)-one;
(4aR,8aS)-6-(3-(3,5-Difluoro-4-(trifluoromethoxy)phenyl)azetidine-1-carbonyl)hexahydro-2H-pyrido[4,3-b][1,4]oxazin-3(4H)-one;
(4aR,8aS)-6-(3-(2-Chloro-4-(trifluoromethoxy)phenyl)azetidine-1-carbonyl)hexahydro-2H-pyrido[4,3-b][1,4]oxazin-3(4H)-one;
(4aR,8aS)-6-(3-(4-(Bicyclo[1.1.1]pentan-1-yl)phenyl)azetidine-1-carbonyl)hexahydro-2H-pyrido[4,3-b][1,4]oxazin-3(4H)-one;
(4aR,8aS)-6-(3-(5-(2-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-Fluoro-1H-indol-3-yl)pyrrolidine-1-carbonyl)hexahydro-2H-pyrido[4,3-b][1,4]oxazin-3(4H)-one;
(4aR,8aS)-6-(3-(2-Fluoro-4-(trifluoromethyl)phenyl)azetidine-1-carbonyl)hexahydro-2H-pyrido[4,3-b][1,4]oxazin-3(4H)-one;
(4aR,8aS)-6-(3-(3-Chloro-4-(trifluoromethoxy)phenyl)azetidine-1-carbonyl)hexahydro-2H-pyrido[4,3-b][1,4]oxazin-3(4H)-one;
(4aR,8aS)-6-(3-(1-Methyl-1H-indazol-4-yl)azetidine-1-carbonyl)hexahydro-2H-pyrido[4,3-b][1,4]oxazin-3(4H)-one;
(4aR,8aS)-6-(3-(4-(3-Fluoropyrrolidin-1-yl)phenyl)azetidine-1-carbonyl)hexahydro-2H-pyrido[4,3-b][1,4]oxazin-3(4H)-one;
(4aR,8aS)-6-[3-[4-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-(3-(1-Methyl-1H-indazol-6-yl)azetidine-1-carbonyl)hexahydro-2H-pyrido[4,3-b][1,4]oxazin-3(4H)-one;
(4aR,8aS)-6-[3-(S or R)-[3-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-[3-(R or S)-[3-(Trifluoromethoxy)phenyl]pyrrolidine-1-carbonyl]-4,4a,5,7,8,8a-hexahydropyrido[4,3-b][1,4]oxazin-3-one;
(4aR,8aS)-6-[3-[4-(Oxetan-3-yl)phenyl]azetidine-1-carbonyl]-4,4a,5,7,8,8a-hexahydropyrido[4,3-b][1,4]oxazin-3-one;
(4aR,8aS)-6-(3-(3-Chloro-4-(3,3-difluoroazetidin-1-yl)phenyl)azetidine-1-carbonyl)hexahydro-2H-pyrido[4,3-b][1,4]oxazin-3(4H)-one;
(4aR,8aS)-6-[(3S or R)-3-(3-Bromophenyl)pyrrolidine-1-carbonyl]-4,4a,5,7,8,8a-hexahydropyrido[4,3-b][1,4]oxazin-3-one;
(4aR,8aS)-6-[(3R or S)-3-(3-Bromophenyl)pyrrolidine-1-carbonyl]-4,4a,5,7,8,8a-hexahydropyrido[4,3-b][1,4]oxazin-3-one;
(4aR,8aS)-6-[3-[4-(3-Azabicyclo[3.1.1]heptan-3-yl)phenyl]azetidine-1-carbonyl]-4,4a,5,7,8,8a-hexahydropyrido[4,3-b][1,4]oxazin-3-one;
(4aR,8aS)-6-(2-Methyl-3-(4-(trifluoromethoxy)phenyl)azetidine-1-carbonyl)hexahydro-2H-pyrido[4,3-b][1,4]oxazin-3(4H)-one;
(4aR,8aS)-6-(3-(3,3-Dimethyl-2,3-dihydrobenzofuran-6-yl)azetidine-1-carbonyl)hexahydro-2H-pyrido[4,3-b][1,4]oxazin-3(4H)-one;
(4aR,8aS)-6-(3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-(3-(3,5-Dichlorophenyl)pyrrolidine-1-carbonyl)hexahydro-2H-pyrido[4,3-b][1,4]oxazin-3(4H)-one;
(4aR,8aS)-6-((R or S)-3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-((S or R)-3-(3-Chloro-5-(2,2,2-trifluoroethoxy)phenyl)pyrrolidine-1-carbonyl)hexahydro-2H-pyrido[4,3-b][1,4]oxazin-3(4H)-one;
(4aR,8aS)-6-(3-(4-(tert-Butyl)-3-methoxyphenyl)azetidine-1-carbonyl)hexahydro-2H-pyrido[4,3-b][1,4]oxazin-3(4H)-one;
(4aR,8aS)-6-(3-(1-Methyl-1H-indazol-5-yl)azetidine-1-carbonyl)hexahydro-2H-pyrido[4,3-b][1,4]oxazin-3(4H)-one;
(4aR,8aS)-6-(3-(4-Propylphenyl)azetidine-1-carbonyl)hexahydro-2H-pyrido[4,3-b][1,4]oxazin-3(4H)-one;
(4aR,8aS)-6-(3-(4-(Trifluoromethoxy)-3-(trifluoromethyl)phenyl)azetidine-1-carbonyl)hexahydro-2H-pyrido[4,3-b][1,4]oxazin-3(4H)-one;
(4aR,8aS)-6-[3-[4-(2,2,2-Trifluoro-1,1-dimethyl-ethyl)phenyl]azetidine-1-carbonyl]-4,4a,5,7,8,8a-hexahydropyrido[4,3-b][1,4]oxazin-3-one;
(4aR,8aS)-6-[3-(R or S)-[4-(2,2,2-Trifluoro-1-methyl-ethoxy)phenyl]azetidine-1-carbonyl]-4,4a,5,7,8,8a-hexahydropyrido[4,3-b][1,4]oxazin-3-one;
(4aR,8aS)-6-[3-[4-(3-Fluoropropyl)phenyl]azetidine-1-carbonyl]-4,4a,5,7,8,8a-hexahydropyrido[4,3-b][1,4]oxazin-3-one;
(4aR,8aS)-6-[3-(S or R)-[4-(2,2,2-Trifluoro-1-methyl-ethoxy)phenyl]azetidine-1-carbonyl]-4,4a,5,7,8,8a-hexahydropyrido[4,3-b][1,4]oxazin-3-one;
(4aR,8aS)-6-[3-(4-Cyclobutylphenyl)azetidine-1-carbonyl]-4,4a,5,7,8,8a-hexahydropyrido[4,3-b][1,4]oxazin-3-one;
(4aR,8aS)-6-[3-(3-Methoxy-4-methyl-phenyl)azetidine-1-carbonyl]-4,4a,5,7,8,8a-hexahydropyrido[4,3-b][1,4]oxazin-3-one;
(4aR,8aS)-6-[3-[5-(2,4-Dichlorophenyl)-1,3,4-oxadiazol-2-yl]azetidine-1-carbonyl]-4,4a,5,7,8,8a-hexahydropyrido[4,3-b][1,4]oxazin-3-one;
(4aR,8aS)-6-[3-[3-Fluoro-4-(trifluoromethyl)phenyl]azetidine-1-carbonyl]-4,4a,5,7,8,8a-hexahydropyrido[4,3-b][1,4]oxazin-3-one;
(4aR,8aS)-6-[3-[1-(2,4-Dichlorophenyl)pyrazol-3-yl]azetidine-1-carbonyl]-4,4a,5,7,8,8a-hexahydropyrido[4,3-b][1,4]oxazin-3-one;
(4aR,8aS)-6-[3-(4-Propoxyphenyl)azetidine-1-carbonyl]-4,4a,5,7,8,8a-hexahydropyrido[4,3-b][1,4]oxazin-3-one;
(4aR,8aS)-6-[3-(3,4-Dimethylphenyl)azetidine-1-carbonyl]-4,4a,5,7,8,8a-hexahydropyrido[4,3-b][1,4]oxazin-3-one;
(4aR,8aS)-6-[3-[4-(2,2-Dimethylpropyl)phenyl]azetidine-1-carbonyl]-4,4a,5,7,8,8a-hexahydropyrido[4,3-b][1,4]oxazin-3-one;
(4aR,8aS)-6-[3-(4-tert-Butoxyphenyl)azetidine-1-carbonyl]-4,4a,5,7,8,8a-hexahydropyrido[4,3-b][1,4]oxazin-3-one;
(4aR,8aS)-6-[4-(5-Chloroindolin-1-yl)piperidine-1-carbonyl]-4,4a,5,7,8,8a-hexahydropyrido[4,3-b][1,4]oxazin-3-one;
(4aR,8aS)-6-[4-(4-Chloroisoindolin-2-yl)piperidine-1-carbonyl]-4,4a,5,7,8,8a-hexahydropyrido[4,3-b][1,4]oxazin-3-one;
(4aR,8aS)-6-[4-(5′-Chlorospiro[cyclopropane-1,3′-indoline]-1′-yl)piperidine-1-carbonyl]-4,4a,5,7,8,8a-hexahydropyrido[4,3-b][1,4]oxazin-3-one;
(4aR,8aS)-6-[3-(4-Chloroisoindolin-2-yl)azetidine-1-carbonyl]-4,4a,5,7,8,8a-hexahydropyrido[4,3-b][1,4]oxazin-3-one;
(4aR,8aS)-6-[4-(5-Chloroisoindolin-2-yl)piperidine-1-carbonyl]-4,4a,5,7,8,8a-hexahydropyrido[4,3-b][1,4]oxazin-3-one;
(4aR,8aS)-6-(3-(5-(3-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-((R or S)-3-(Trifluoromethyl)pyrrolidin-1-yl)pyridin-2-yl)azetidine-1-carbonyl)hexahydro-2H-pyrido[4,3-b][1,4]oxazin-3(4H)-one;
(4aR,8aS)-6-(3-(5-((S or R)-3-(Trifluoromethyl)pyrrolidin-1-yl)pyridin-2-yl)azetidine-1-carbonyl)hexahydro-2H-pyrido[4,3-b][1,4]oxazin-3(4H)-one;
rac-(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
(4aR,8aS)-6-[3-[6-[3-(trifluoromethyl)pyrrolidin-1-yl]-3-pyridyl]azetidine-1-carbonyl]-4,4a,5,7,8,8a-hexahydropyrido[4,3-b][1,4]oxazin-3-one;
rac-(4aR,8aS)-6-[3-(4-tetrahydropyran-3-ylphenyl)azetidine-1-carbonyl]-4,4a,5,7,8,8a-hexahydropyrido[4,3-b][1,4]oxazin-3-one;
(4aR,8aS)-6-[3-[2-methoxy-4-(2,2,2-trifluoroethyl)phenyl]azetidine-1-carbonyl]-4,4a,5,7,8,8a-hexahydropyrido[4,3-b][1,4]oxazin-3-one;
(4aR,8aS)-6-[3-[4-(2,2-dimethylpropoxy)phenyl]azetidine-1-carbonyl]-4,4a,5,7,8,8a-hexahydropyrido[4,3-b][1,4]oxazin-3-one;
(4aR,8aS)-6-[4-[[2-(2,2,2-trifluoroethoxy)-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[3-[4-chloro-3-(trifluoromethyl)phenoxy]azetidine-1-carbonyl]-4,4a,5,7,8,8a-hexahydropyrido[4,3-b][1,4]oxazin-3-one;
(4aR,8aS)-6-[4-[3-morpholino-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-[3-cyclopropyl-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-(4-chlorophenoxy)piperidine-1-carbonyl]-4,4a,5,7,8,8a-hexahydropyrido[4,3-b][1,4]oxazin-3-one;
(4aR,8aS)-6-[4-[[2,6-difluoro-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[4-chloro-3-(4-chlorophenyl)-2-fluoro-phenoxy]piperidine-1-carbonyl]-4,4a,5,7,8,8a-hexahydropyrido[4,3-b][1,4]oxazin-3-one;
(4aR,8aS)-6-[3-[2-chloro-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[[2-fluoro-6-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-methyl-2-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[6-fluoro-4-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-[[6-fluoro-5-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-[(3,4-dichlorophenyl)methoxy]azetidine-1-carbonyl]-4,4a,5,7,8,8a-hexahydropyrido[4,3-b][1,4]oxazin-3-one;
(4aR,8aS)-6-[3-[(2,5-dichlorophenyl)methoxy]azetidine-1-carbonyl]-4,4a,5,7,8,8a-hexahydropyrido[4,3-b][1,4]oxazin-3-one;
(4aR,8aS)-6-[3-[[3-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[5-(trifluoromethyl)-2-pyridyl]oxymethyl]azetidine-1-carbonyl]-4,4a,5,7,8,8a-hexahydropyrido[4,3-b][1,4]oxazin-3-one;
(4aR,8aS)-6-[3-methyl-4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[3-methyl-4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[4,5-bis(trifluoromethyl)-2-pyridyl]oxymethyl]azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[2-fluoro-4-(pentafluoro-lambda6-sulfanyl)phenyl]methoxy]azetidine-1-carbonyl]-4,4a,5,7,8,8a-hexahydropyrido[4,3-b][1,4]oxazin-3-one;
(4aR,8aS)-6-[3-[[4-(4-fluorophenyl)thiazol-2-yl]methoxy]azetidine-1-carbonyl]-4,4a,5,7,8,8a-hexahydropyrido[4,3-b][1,4]oxazin-3-one;
rac-(4aR,8aS)-6-[rac-(2R,3S)-3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
(4aR,8aS)-6-[3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
(4aR,8aS)-6-[3-[2-bromo-5-(trifluoromethyl)phenoxy]-2-methyl-pyrrolidine-1-carbonyl]-4,4a,5,7,8,8a-hexahydropyrido[4,3-b][1,4]oxazin-3-one;
6-[3-[[2,4-bis(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methyl-4-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[2-methyl-4-(trifluoromethoxy)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[2-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[2-fluoro-4-(trifluoromethyl)phenoxy]piperidine-1-carbonyl]-4,4a,5,7,8,8a-hexahydropyrido[4,3-b][1,4]oxazin-3-one;
(4aR,8aS)-6-[4-[3-chloro-4-(trifluoromethyl)phenoxy]piperidine-1-carbonyl]-4,4a,5,7,8,8a-hexahydropyrido[4,3-b][1,4]oxazin-3-one;
(4aR,8aS)-6-[3-(4-chloro-3-cyclopropyl-phenoxy)azetidine-1-carbonyl]-4,4a,5,7,8,8a-hexahydropyrido[4,3-b][1,4]oxazin-3-one;
(4aR,8aS)-6-[4-[2-chloro-3-(trifluoromethyl)phenoxy]piperidine-1-carbonyl]-4,4a,5,7,8,8a-hexahydropyrido[4,3-b][1,4]oxazin-3-one;
(4aR,8aS)-6-[3-(3-bromo-2-chloro-phenoxy)azetidine-1-carbonyl]-4,4a,5,7,8,8a-hexahydropyrido[4,3-b][1,4]oxazin-3-one;
(4aR,8aS)-6-[3-(2-chloro-3-cyclopropyl-phenoxy)azetidine-1-carbonyl]-4,4a,5,7,8,8a-hexahydropyrido[4,3-b][1,4]oxazin-3-one;
(4aR,8aS)-6-[3-[3-cyclopropyl-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-chloro-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-(3-bromo-4-chloro-phenoxy)azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(3,3-difluoroazetidin-1-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(6,6-difluoro-2-azaspiro[3.3]heptan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(5-oxa-2-azaspiro[3.5]nonan-2-yl)-4-(trifluoromethyl)phenoxy]azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-2-chloro-phenoxy]azetidine-1-carbonyl]-4,4a,5,7,8,8a-hexahydropyrido[4,3-b][1,4]oxazin-3-one;
(4aR,8aS)-6-[3-[2-chloro-3-(5-oxa-2-azaspiro[3.4]octan-2-yl)phenoxy]azetidine-1-carbonyl]-4,4a,5,7,8,8a-hexahydropyrido[4,3-b][1,4]oxazin-3-one;
(4aR,8aS)-6-[3-[2-chloro-3-(5-oxa-2-azaspiro[3.5]nonan-2-yl)phenoxy]azetidine-1-carbonyl]-4,4a,5,7,8,8a-hexahydropyrido[4,3-b][1,4]oxazin-3-one;
(4aR,8aS)-6-[3-[3-(2-azaspiro[3.3]heptan-2-yl)-5-chloro-phenoxy]azetidine-1-carbonyl]-4,4a,5,7,8,8a-hexahydropyrido[4,3-b][1,4]oxazin-3-one;
(4aR,8aS)-6-[3-(3-chloro-5-pyrrolidin-1-yl-phenoxy)azetidine-1-carbonyl]-4,4a,5,7,8,8a-hexahydropyrido[4,3-b][1,4]oxazin-3-one;
(4aR,8aS)-6-[3-[[4-fluoro-2-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-(trifluoromethoxy)phenyl]methyl]azetidine-1-carbonyl]-4,4a,5,7,8,8a-hexahydropyrido[4,3-b][1,4]oxazin-3-one;
rac-(4aR,8aS)-6-[3-[2-fluoro-5-(trifluoromethyl)phenoxy]pyrrolidine-1-carbonyl]-4,4a,5,7,8,8a-hexahydropyrido[4,3-b][1,4]oxazin-3-one;
rac-(4aR,8aS)-6-[3-[2-chloro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[(3S)-3-[2-fluoro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[(3R)-3-[2-fluoro-5-(trifluoromethyl)phenoxy]pyrrolidine-1-carbonyl]-4,4a,5,7,8,8a-hexahydropyrido[4,3-b][1,4]oxazin-3-one;
(4aR,8aS)-6-[3-[[3-fluoro-4-(trifluoromethoxy)phenyl]methoxy]azetidine-1-carbonyl]-4,4a,5,7,8,8a-hexahydropyrido[4,3-b][1,4]oxazin-3-one;
(4aR,8aS)-6-[3-[(2,3-dimethylphenyl)methoxy]azetidine-1-carbonyl]-4,4a,5,7,8,8a-hexahydropyrido[4,3-b][1,4]oxazin-3-one;
(4aR,8aS)-6-[3-[(2,4-dimethylphenyl)methoxy]azetidine-1-carbonyl]-4,4a,5,7,8,8a-hexahydropyrido[4,3-b][1,4]oxazin-3-one;
(4aR,8aS)-6-[3-[[2-methyl-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-methyl-3-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[(4-tert-butylthiazol-2-yl)methyl]piperidine-1-carbonyl]-4,4a,5,7,8,8a-hexahydropyrido[4,3-b][1,4]oxazin-3-one;
(4aR,8aS)-6-[4-[(4-tert-butyloxazol-2-yl)methyl]piperidine-1-carbonyl]-4,4a,5,7,8,8a-hexahydropyrido[4,3-b][1,4]oxazin-3-one;
rac-(4aR,8aS)-6-[4-[(4-tert-butylthiazol-2-yl)methyl]piperidine-1-carbonyl]-4,4a,5,7,8,8a-hexahydropyrido[4,3-b][1,4]oxazin-3-one;
rac-(4aR,8aS)-6-[4-[(4-tert-butyloxazol-2-yl)methyl]piperidine-1-carbonyl]-4,4a,5,7,8,8a-hexahydropyrido[4,3-b][1,4]oxazin-3-one;
(4aS,8aR)-6-[4-[(4-tert-butylthiazol-2-yl)methyl]piperidine-1-carbonyl]-4,4a,5,7,8,8a-hexahydropyrido[4,3-b][1,4]oxazin-3-one;
(4aS,8aR)-6-[4-[(4-tert-butyloxazol-2-yl)methyl]piperidine-1-carbonyl]-4,4a,5,7,8,8a-hexahydropyrido[4,3-b][1,4]oxazin-3-one;
rac-(4aR,8aS)-6-[4-[(2-chloro-4-fluoro-phenoxy)methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[(4-chlorophenoxy)methyl]piperidine-1-carbonyl]-4,4a,5,7,8,8a-hexahydropyrido[4,3-b][1,4]oxazin-3-one;
rac-(4aR,8aS)-6-[4-[(4-chlorophenyl)methyl]piperidine-1-carbonyl]-4,4a,5,7,8,8a-hexahydropyrido[4,3-b][1,4]oxazin-3-one;
rac-(4aR,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[4-(trifluoromethyl)phenoxy]piperidine-1-carbonyl]-4,4a,5,7,8,8a-hexahydropyrido[4,3-b][1,4]oxazin-3-one;
rac-(4aR,8aS)-6-[4-[4-(trifluoromethyl)phenoxy]piperidine-1-carbonyl]-4,4a,5,7,8,8a-hexahydropyrido[4,3-b][1,4]oxazin-3-one;
(4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-(phenoxymethyl)piperidine-1-carbonyl]-4,4a,5,7,8,8a-hexahydropyrido[4,3-b][1,4]oxazin-3-one;
rac-(4aR,8aS)-6-[4-(5,6-dihydro-4H-cyclopenta[d]thiazol-2-ylmethyl)piperidine-1-carbonyl]-4,4a,5,7,8,8a-hexahydropyrido[4,3-b][1,4]oxazin-3-one;
rac-(4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[(3-phenyl-1,2,4-oxadiazol-5-yl)methyl]piperidine-1-carbonyl]-4,4a,5,7,8,8a-hexahydropyrido[4,3-b][1,4]oxazin-3-one;
(4aR,8aR)-6-[4-[[4-(trifluoromethyl)phenyl]methyl]piperidine-1-carbonyl]-4,4a,5,7,8,8a-hexahydropyrido[4,3-b][1,4]oxazin-3-one;
(4aS,8aR)-6-[4-[[4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[4-[[4-(trifluoromethyl)phenyl]methyl]piperazine-1-carbonyl]-4,4a,5,7,8,8a-hexahydropyrido[4,3-b][1,4]oxazin-3-one;
rac-(4aR,8aS)-6-[4-[(4-chlorophenyl)methyl]piperazine-1-carbonyl]-4,4a,5,7,8,8a-hexahydropyrido[4,3-b][1,4]oxazin-3-one;
(4aS,8aR)-6-[4-[(2-chloro-4-fluoro-phenoxy)methyl]piperidine-1-carbonyl]-4,4a,5,7,8,8a-hexahydropyrido[4,3-b][1,4]oxazin-3-one;
(4aR,8aS)-6-[4-[(2-chloro-4-fluoro-phenoxy)methyl]piperidine-1-carbonyl]-4,4a,5,7,8,8a-hexahydropyrido[4,3-b][1,4]oxazin-3-one;
rac-(4aR,8aS)-6-[4-[[5-(trifluoromethyl)-2-pyridyl]methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)pyrazol-1-yl]methyl]piperidine-1-carbonyl]-4,4a,5,7,8,8a-hexahydropyrido[4,3-b][1,4]oxazin-3-one;
rac-(4aR,8aS)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[(2-chloro-4-fluoro-phenyl)methoxy]azetidine-1-carbonyl]-4,4a,5,7,8,8a-hexahydropyrido[4,3-b][1,4]oxazin-3-one;
(4aR,8aS)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aS,8aR)-6-[3-[[2-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[[4-(trifluoromethoxy)phenyl]methyl]piperidine-1-carbonyl]-4,4a,5,7,8,8a-hexahydropyrido[4,3-b][1,4]oxazin-3-one;
(4aR,8aS)-6-[4-[(2,4-difluorophenoxy)methyl]piperidine-1-carbonyl]-4,4a,5,7,8,8a-hexahydropyrido[4,3-b][1,4]oxazin-3-one;
(4aR,8aS)-6-[4-[(4-chloro-3-fluoro-phenyl)methyl]piperidine-1-carbonyl]-4,4a,5,7,8,8a-hexahydropyrido[4,3-b][1,4]oxazin-3-one;
(4aR,8aS)-6-[4-[(4-chlorophenyl)methyl]piperidine-1-carbonyl]-4,4a,5,7,8,8a-hexahydropyrido[4,3-b][1,4]oxazin-3-one;
(4aR,8aS)-6-[3-[[4-(trifluoromethyl)phenyl]methyl]azetidine-1-carbonyl]-4,4a,5,7,8,8a-hexahydropyrido[4,3-b][1,4]oxazin-3-one;
(4aR,8aS)-6-[4-[(4,4-difluoro-1-piperidyl)methyl]piperidine-1-carbonyl]-4,4a,5,7,8,8a-hexahydropyrido[4,3-b][1,4]oxazin-3-one;
(4aR,8aS)-6-[4-[(5-tert-butyloxazol-2-yl)methyl]piperidine-1-carbonyl]-4,4a,5,7,8,8a-hexahydropyrido[4,3-b][1,4]oxazin-3-one;
(4aR,8aS)-6-[4-[(2-fluoro-4-methoxy-phenoxy)methyl]piperidine-1-carbonyl]-4,4a,5,7,8,8a-hexahydropyrido[4,3-b][1,4]oxazin-3-one;
(4aR,8aS)-6-[4-[2-chloro-4-(trifluoromethyl)phenoxy]piperidine-1-carbonyl]-4,4a,5,7,8,8a-hexahydropyrido[4,3-b][1,4]oxazin-3-one;
(4aS,8aR)-6-[4-[[6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[4-[[6-(trifluoromethyl)-3-pyridyl]oxymethyl]piperidine-1-carbonyl]-4,4a,5,7,8,8a-hexahydropyrido[4,3-b][1,4]oxazin-3-one;
(4aR,8aS)-6-[4-[3-(trifluoromethyl)phenoxy]piperidine-1-carbonyl]-4,4a,5,7,8,8a-hexahydropyrido[4,3-b][1,4]oxazin-3-one;
(4aS,8aR)-6-[4-[[2-chloro-4-(trifluoromethoxy)phenoxy]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethoxy)phenoxy]methyl]piperidine-1-carbonyl]-4,4a,5,7,8,8a-hexahydropyrido[4,3-b][1,4]oxazin-3-one;
(4aR,8aS)-6-[3-[[2-fluoro-4-(trifluoromethyl)phenoxy]methyl]azetidine-1-carbonyl]-4,4a,5,7,8,8a-hexahydropyrido[4,3-b][1,4]oxazin-3-one;
(4aS,8aR)-6-[4-[(2,4-difluorophenoxy)methyl]piperidine-1-carbonyl]-4,4a,5,7,8,8a-hexahydropyrido[4,3-b][1,4]oxazin-3-one;
(4aR,8aS)-6-[4-[(4-chloro-2-fluoro-phenoxy)methyl]piperidine-1-carbonyl]-4,4a,5,7,8,8a-hexahydropyrido[4,3-b][1,4]oxazin-3-one;
(4aR,8aS)-6-[4-[[4-fluoro-2-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[4-[[2-fluoro-4-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[4-[[2-pyrrolidin-1-yl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethyl)phenoxy]methyl]piperidine-1-carbonyl]-4,4a,5,7,8,8a-hexahydropyrido[4,3-b][1,4]oxazin-3-one;
(4aR,8aS)-6-[3-[(2-chloro-4-fluoro-phenoxy)methyl]azetidine-1-carbonyl]-4,4a,5,7,8,8a-hexahydropyrido[4,3-b][1,4]oxazin-3-one;
(4aR,8aS)-6-[4-[[2-cyclopentyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[4-(trifluoromethyl)imidazol-1-yl]methyl]piperidine-1-carbonyl]-4,4a,5,7,8,8a-hexahydropyrido[4,3-b][1,4]oxazin-3-one;
(4aR,8aS)-6-[4-[(4-fluoro-2-methyl-phenoxy)methyl]piperidine-1-carbonyl]-4,4a,5,7,8,8a-hexahydropyrido[4,3-b][1,4]oxazin-3-one;
(4aR,8aS)-6-[3-[[2-chloro-4-(trifluoromethyl)phenoxy]methyl]azetidine-1-carbonyl]-4,4a,5,7,8,8a-hexahydropyrido[4,3-b][1,4]oxazin-3-one;
(4aR,8aS)-6-[4-[(4-tert-butylpyrazol-1-yl)methyl]piperidine-1-carbonyl]-4,4a,5,7,8,8a-hexahydropyrido[4,3-b][1,4]oxazin-3-one;
(2R,4aR,8aS)-2-methyl-6-[4-[[4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-(1,3-benzoxazol-2-ylmethyl)piperidine-1-carbonyl]-4,4a,5,7,8,8a-hexahydropyrido[4,3-b][1,4]oxazin-3-one;
(4aR,8aS)-6-[4-[4-chloro-3-(4-chlorophenyl)phenoxy]piperidine-1-carbonyl]-4,4a,5,7,8,8a-hexahydropyrido[4,3-b][1,4]oxazin-3-one;
(4aR,8aS)-6-[4-[(2-chloro-4-fluoro-phenoxy)methyl]-3-methyl-piperidine-1-carbonyl]-4,4a,5,7,8,8a-hexahydropyrido[4,3-b][1,4]oxazin-3-one;
(4aR,8aS)-6-[3-[[2-chloro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-fluoro-4-(trifluoromethoxy)phenyl]methoxy]azetidine-1-carbonyl]-4,4a,5,7,8,8a-hexahydropyrido[4,3-b][1,4]oxazin-3-one;
(4aR,8aS)-6-[4-[[2-(1H-pyrazol-4-yl)-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[3-[(2,4-dichlorophenyl)methoxy]azetidine-1-carbonyl]-4,4a,5,7,8,8a-hexahydropyrido[4,3-b][1,4]oxazin-3-one;
(4aR,8aS)-6-[3-[[3-methoxy-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[4-[[5-methyl-6-(trifluoromethyl)-3-pyridyl]oxymethyl]piperidine-1-carbonyl]-4,4a,5,7,8,8a-hexahydropyrido[4,3-b][1,4]oxazin-3-one;
rac-(4aR,8aS)-6-[3-[(3-chlorophenoxy)methyl]pyrrolidine-1-carbonyl]-4,4a,5,7,8,8a-hexahydropyrido[4,3-b][1,4]oxazin-3-one;
rac-(4aR,8aS)-6-[3-[(2-chlorophenoxy)methyl]pyrrolidine-1-carbonyl]-4,4a,5,7,8,8a-hexahydropyrido[4,3-b][1,4]oxazin-3-one;
(4aR,8aS)-6-[4-[[2-fluoro-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[(2-chlorophenyl)methoxy]pyrrolidine-1-carbonyl]-4,4a,5,7,8,8a-hexahydropyrido[4,3-b][1,4]oxazin-3-one;
rac-(4aR,8aS)-6-[3-[(3-chlorophenyl)methoxy]pyrrolidine-1-carbonyl]-4,4a,5,7,8,8a-hexahydropyrido[4,3-b][1,4]oxazin-3-one;
(4aR,8aS)-6-[4-[[2-cyclopropyl-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[(4-chlorophenoxy)methyl]pyrrolidine-1-carbonyl]-4,4a,5,7,8,8a-hexahydropyrido[4,3-b][1,4]oxazin-3-one;
rac-(4aR,8aS)-6-[3-[(4-chlorophenyl)methoxy]pyrrolidine-1-carbonyl]-4,4a,5,7,8,8a-hexahydropyrido[4,3-b][1,4]oxazin-3-one;
(4aR,8aS)-6-[4-[[2-methyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2-chloro-4-(trifluoromethyl)phenyl]methyl]piperidine-1-carbonyl]-4,4a,5,7,8,8a-hexahydropyrido[4,3-b][1,4]oxazin-3-one;
rac-(4aR,8aS)-6-[3-[[4-(trifluoromethyl)phenyl]methyl]pyrrolidine-1-carbonyl]-4,4a,5,7,8,8a-hexahydropyrido[4,3-b][1,4]oxazin-3-one;
(4aR,8aS)-6-[3-[[3-fluoro-5-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-chloro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2,4-difluoro-5-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-fluoro-5-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[3-fluoro-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[2-methoxy-4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[[4-chloro-2-(trifluoromethyl)phenyl]methoxy]azetidine-1-carbonyl]-4,4a,5,7,8,8a-hexahydropyrido[4,3-b][1,4]oxazin-3-one;
rac-(4aR,8aS)-6-[2-methyl-3-[[4-(trifluoromethyl)phenyl]methoxy]azetidine-1-carbonyl]-4,4a,5,7,8,8a-hexahydropyrido[4,3-b][1,4]oxazin-3-one;
(4aR,8aS)-6-[3-[4-(trifluoromethyl)phenoxy]azetidine-1-carbonyl]-4,4a,5,7,8,8a-hexahydropyrido[4,3-b][1,4]oxazin-3-one;
(4aR,8aS)-6-[4-[4-chloro-3-(trifluoromethyl)phenoxy]piperidine-1-carbonyl]-4,4a,5,7,8,8a-hexahydropyrido[4,3-b][1,4]oxazin-3-one;
(4aR,8aS)-6-[4-(4-chloro-3-cyclopropyl-phenoxy)piperidine-1-carbonyl]-4,4a,5,7,8,8a-hexahydropyrido[4,3-b][1,4]oxazin-3-one;
(4aR,8aS)-6-[4-(4-chloro-3-morpholino-phenoxy)piperidine-1-carbonyl]-4,4a,5,7,8,8a-hexahydropyrido[4,3-b][1,4]oxazin-3-one;
(4aR,8aS)-6-[4-[2-methyl-4-(trifluoromethyl)phenoxy]piperidine-1-carbonyl]-4,4a,5,7,8,8a-hexahydropyrido[4,3-b][1,4]oxazin-3-one; or
(4aR,8aS)-6-[4-(oxazolo[5,4-c]pyridin-2-ylmethyl)piperidine-1-carbonyl]-4,4a,5,7,8,8a-hexahydropyrido[4,3-b][1,4]oxazin-3-one.

43. The compound of claim 42, or a pharmaceutically acceptable salt thereof, wherein X is N.

44. The compound of claim 42, or a pharmaceutically acceptable salt thereof, wherein:
R² is a group 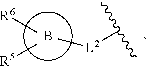
C₁-C₆-alkyl, C₁-C₆-alkoxy, halo-C₁-C₆-alkyl, halo-C₁-C₆-alkoxy, halogen, or SF₅;
R³ is hydrogen, halogen, C₁-C₆-alkyl, or halo-C₁-C₆-alkyl;
R⁴ is hydrogen;
B is
(i) C₆-C₁₄-aryl or 3-14 membered heterocyclyl and L² is a covalent bond; or
(ii) C₃-C₁₀-cycloalkyl and L² is a covalent bond, —O—, or —CH₂O—; and
R⁶ is hydrogen or halogen.

45. The compound of claim 42, or a pharmaceutically acceptable salt thereof, wherein A is C₆-C₁₄-aryl.

46. The compound of claim 44, or a pharmaceutically acceptable salt thereof, wherein A is C₆-C₁₄-aryl.

47. The compound claim 42, or a pharmaceutically acceptable salt thereof, wherein:
R² is a group 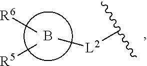
halo-C₁-C₆-alkyl, halo-C₁-C₆-alkoxy, halogen, or SF₅;
R⁴ is hydrogen;
R³ is hydrogen, halogen, or halo-C₁-C₆-alkyl;
B is
(i) 3-14 membered heterocyclyl and L² is a covalent bond; or
(ii) C₃-C₁₀-cycloalkyl and L² is a covalent bond, —O—, or —CH₂O—; and
R⁵ and R⁶ are independently selected from hydrogen and halogen.

48. The compound of claim 42, or a pharmaceutically acceptable salt thereof, wherein A is phenyl.

49. The compound of claim 47, or a pharmaceutically acceptable salt thereof, wherein A is phenyl.

50. The compound of claim 42, or a pharmaceutically acceptable salt thereof, wherein:
R² is a group 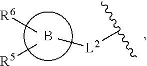
CF₃, OCF₃, 2,2,2-trifluoro-1,1-dimethyl-ethoxy, fluoro, or SF₅;
R³ is hydrogen, chloro, or CF₃;
R⁴ is hydrogen;
B is
(i) tetrahydropyranyl and L² is a covalent bond; or
(ii) cyclopropyl or cyclobutyl and L² is a covalent bond, —O—, and —CH₂O—; and
R⁵ and R⁶ are independently selected from hydrogen and fluoro.

51. The compound of claim 42, or a pharmaceutically acceptable salt thereof, wherein L¹ is a covalent bond, —CH₂—, —OCHR^{L}—, —CH₂O—, or —NHC(O)—.

52. The compound of claim 42, or a pharmaceutically acceptable salt thereof, wherein L¹ is a covalent bond, —OCH₂—, —CH₂O—, or —CH₂—.

53. The compound of claim 42, or a pharmaceutically acceptable salt thereof, wherein R¹ is hydrogen or methyl.

54. The compound of claim 46, or a pharmaceutically acceptable salt thereof, wherein L¹ is a covalent bond, —CH₂—, —OCHR^{L}—, —CH₂O—, or —NHC(O)—; R¹ is hydrogen or methyl; and X is N.

55. The compound of claim 49, or a pharmaceutically acceptable salt thereof, wherein L¹ is a covalent bond, —CH₂—, —OCHR^{L}—, —CH₂O—, or —NHC(O)—; R¹ is hydrogen or methyl; and X is N.

56. The compound of claim 50, or a pharmaceutically acceptable salt thereof, wherein L¹ is a covalent bond, —CH₂—, —OCHR^{L}—, —CH₂O—, or —NHC(O)—; R¹ is hydrogen or methyl; and X is N.

57. The compound of claim 42, wherein the compound is:
(+)- or (−)-(4aR,8aS)-6-[3-(4-Tetrahydrofuran-3-ylphenyl)azetidine-1-carbonyl]-4,4a,5,7,8,8a-hexahydropyrido[4,3-b][1,4]oxazin-3-one;
(4aR,8aS)-6-[3-[4-(3,3-Difluorocyclobutoxy)phenyl]azetidine-1-carbonyl]-4,4a,5,7,8,8a-hexahydropyrido[4,3-b][1,4]oxazin-3-one;
(4aR,8aS)-6-[3-(4-Isopropoxyphenyl)azetidine-1-carbonyl]-4,4a,5,7,8,8a-hexahydropyrido[4,3-b][1,4]oxazin-3-one;
(4aR,8aS)-6-[3-(4-tetrahydropyran-4-ylphenyl)azetidine-1-carbonyl]-4,4a,5,7,8,8a-hexahydropyrido[4,3-b][1,4]oxazin-3-one;
(4aR,8aS)-6-[3-[4-(3,3-difluorocyclobutyl)phenyl]azetidine-1-carbonyl]-4,4a,5,7,8,8a-hexahydropyrido[4,3-b][1,4]oxazin-3-one;
(4aR,8aS)-6-[3-[5-(2,4-dichlorophenyl)-1,2,4-thiadiazol-3-yl]azetidine-1-carbonyl]-4,4a,5,7,8,8a-hexahydropyrido[4,3-b][1,4]oxazin-3-one;
(4aR,8aS)-6-[3-[1-(2,4-dichlorophenyl)imidazol-4-yl]azetidine-1-carbonyl]-4,4a,5,7,8,8a-hexahydropyrido[4,3-b][1,4]oxazin-3-one;
(4aR,8aS)-6-[3-[3-[4-chloro-2-(trifluoromethyl)phenyl]-1,2,4-oxadiazol-5-yl]azetidine-1-carbonyl]-4,4a,5,7,8,8a-hexahydropyrido[4,3-b][1,4]oxazin-3-one;
(4aR,8aS)-6-(3-(2,2-dimethylchroman-6-yl)azetidine-1-carbonyl)hexahydro-2H-pyrido[4,3-b][1,4]oxazin-3(4H)-one;
(4aR,8aS)-6-(3-(4-isobutoxyphenyl)azetidine-1-carbonyl)hexahydro-2H-pyrido[4,3-b][1,4]oxazin-3(4H)-one;
(4aR,8aS)-6-[3-[4-(2,2,2-trifluoro-1,1-dimethyl-ethoxy)phenyl]azetidine-1-carbonyl]-4,4a,5,7,8,8a-hexahydropyrido[4,3-b][1,4]oxazin-3-one;
(4aR,8aS)-6-(3-(4-(cyclopentyloxy)phenyl)azetidine-1-carbonyl)hexahydro-2H-pyrido[4,3-b][1,4]oxazin-3(4H)-one;
(4aR,8aS)-6-(3-(4,4-Dimethylchroman-6-yl)azetidine-1-carbonyl)hexahydro-2H-pyrido[4,3-b][1,4]oxazin-3(4H)-one;
(4aR,8aS)-6-(3-(4-(Cyclopropylmethoxy)phenyl)azetidine-1-carbonyl)hexahydro-2H-pyrido[4,3-b][1,4]oxazin-3(4H)-one;
(4aR,8aS)-6-[3-[3-(2,4-dichlorophenyl)-1,2,4-oxadiazol-5-yl]azetidine-1-carbonyl]-4,4a,5,7,8,8a-hexahydropyrido[4,3-b][1,4]oxazin-3-one;
(4aR,8aS)-6-(3-(1-methyl-1H-indazol-5-yl)azetidine-1-carbonyl)hexahydro-2H-pyrido[4,3-b][1,4]oxazin-3(4H)-one;
(4aR,8aS)-6-(3-(5-(2,4-dichlorophenyl)-1,2,4-oxadiazol-3-yl)azetidine-1-carbonyl)hexahydro-2H-pyrido[4,3-b][1,4]oxazin-3(4H)-one;
(4aR,8aS)-6-(3-(3-(trifluoromethoxy)phenyl)azetidine-1-carbonyl)hexahydro-2H-pyrido[4,3-b][1,4]oxazin-3(4H)-one;
(4aR,8aS)-6-(4-(1-(4-fluorophenyl)-1H-pyrazol-3-yl)piperidine-1-carbonyl)hexahydro-2H-pyrido[4,3-b][1,4]oxazin-3(4H)-one;
(4aR,8aS)-6-(3-((2,6-Dichlorobenzyl)oxy)azetidine-1-carbonyl)hexahydro-2H-pyrido[4,3-b][1,4]oxazin-3(4H)-one;
(4aR,8aS)-6-(3-((3,5-dichlorobenzyl)oxy)azetidine-1-carbonyl)hexahydro-2H-pyrido[4,3-b][1,4]oxazin-3(4H)-one;
(4aR,8aS)-6-(4-((5-cyclopropyl-4-methylpyridin-3-yl)methyl)piperidine-1-carbonyl)hexahydro-2H-pyrido[4,3-b][1,4]oxazin-3(4H)-one;
(4aR,8aS)-6-(4-((4-(trifluoromethyl)benzyl)oxy)piperidine-1-carbonyl)hexahydro-2H-pyrido[4,3-b][1,4]oxazin-3(4H)-one;
(4aR,8aS)-6-(4-((2-chloro-4-fluorobenzyl)oxy)piperidine-1-carbonyl)hexahydro-2H-pyrido[4,3-b][1,4]oxazin-3(4H)-one;
(4aR,8aS)-6-(4-((2-Chloro-4-(trifluoromethyl)benzyl)oxy)piperidine-1-carbonyl)hexahydro-2H-pyrido[4,3-b][1,4]oxazin-3(4H)-one;
rac-(4aR,8aS)-6-[3-[[2-Fluoro-6-(trifluoromethyl)phenyl]methoxy]-2-methyl-azetidine-1-carbonyl]-4,4a,5,7,8,8a-hexahydropyrido[4,3-b][1,4]oxazin-3-one;
rac-(4aR,8aS)-6-[3-[[2-Fluoro-6-(trifluoromethyl)phenyl]methoxy]-2-methyl-azetidine-1-carbonyl]-4,4a,5,7,8,8a-hexahydropyrido[4,3-b][1,4]oxazin-3-one;
(4aR,8aS)-6-[3-[[2-Fluoro-6-(trifluoromethyl)phenyl]methoxy]-2-methylazetidine-1-carbonyl]-4,4a,5,7,8,8a-hexahydropyrido[4,3-b][1,4]oxazin-3-one CHECK!;
(4aR,8aS)-6-(3-((4-fluoro-3-methoxybenzyl)oxy)azetidine-1-carbonyl)hexahydro-2H-pyrido[4,3-b][1,4]oxazin-3(4H)-one;
(4aR,8aS)-6-(3-((4-(pentafluoro-16-sulfaneyl)benzyl)oxy)azetidine-1-carbonyl)hexahydro-2H-pyrido[4,3-b][1,4]oxazin-3(4H)-one;
(4aR,8aS)-6-(3-((2-(trifluoromethoxy)benzyl)oxy)azetidine-1-carbonyl)hexahydro-2H-pyrido[4,3-b][1,4]oxazin-3(4H)-one;
(4aR,8aS)-6-(3-((4-chloro-2-(trifluoromethoxy)benzyl)oxy)azetidine-1-carbonyl)hexahydro-2H-pyrido[4,3-b][1,4]oxazin-3(4H)-one;
(4aR,8aS)-6-(3-((2-methyl-5-(trifluoromethyl)benzyl)oxy)azetidine-1-carbonyl)hexahydro-2H-pyrido[4,3-b][1,4]oxazin-3(4H)-one;
(4aR,8aS)-6-[4-[[3-phenyl-4-(trifluoromethyl)phenyl]methyl]piperidine-1-carbonyl]-4,4a,5,7,8,8a-hexahydropyrido[4,3-b][1,4]oxazin-3-one;
(4aR,8aS)-6-[4-[[2,4-bis(trifluoromethyl)phenyl]methyl]piperidine-1-carbonyl]-4,4a,5,7,8,8a-hexahydropyrido[4,3-b][1,4]oxazin-3-one;
(4aR,8aS)-6-(4-((5-methyl-6-(trifluoromethyl)pyridin-3-yl)methyl)piperidine-1-carbonyl)hexahydro-2H-pyrido[4,3-b][1,4]oxazin-3(4H)-one;
(4aR,8aS)-6-(4-((2-chloro-4-fluorophenoxy)methyl)-3-methylpiperidine-1-carbonyl)hexahydro-2H-pyrido[4,3-b][1,4]oxazin-3(4H)-one;
(4aR,8aS)-6-(3-((4-(difluoromethoxy)benzyl)oxy)azetidine-1-carbonyl)hexahydro-2H-pyrido[4,3-b][1,4]oxazin-3(4H)-one;
(4aR,8aS)-6-(3-((2-chloro-4-(trifluoromethyl)benzyl)oxy)cyclobutane-1-carbonyl)hexahydro-2H-pyrido[4,3-b][1,4]oxazin-3(4H)-one;
(4aR,8aS)-6-(3-((5-(trifluoromethyl)pyridin-2-yl)methoxy)azetidine-1-carbonyl)hexahydro-2H-pyrido[4,3-b][1,4]oxazin-3(4H)-one;
(4aR,8aS)-6-(3-((4-(trifluoromethoxy)benzyl)oxy)azetidine-1-carbonyl)hexahydro-2H-pyrido[4,3-b][1,4]oxazin-3(4H)-one;
N-(2-chloro-4-fluorophenyl)-1-((4aR,8aS)-3-oxooctahydro-2H-pyrido[4,3-b][1,4]oxazine-6-carbonyl)azetidine-3-carboxamide;
(4aS,8aR)-6-(3-(1-(2-chloro-4-(trifluoromethyl)phenoxy)ethyl)azetidine-1-carbonyl)hexahydro-2H-pyrido[4,3-b][1,4]oxazin-3(4H)-one;
(4aR,8aS)-6-(4-(3-(trifluoromethyl)pyridazin-4-yl)piperidine-1-carbonyl)hexahydro-2H-pyrido[4,3-b][1,4]oxazin-3(4H)-one;
(4aR,8aS)-6-[3-[5-(2,4-difluorophenyl)-4H-1,2,4-triazol-3-yl]azetidine-1-carbonyl]-4,4a,5,7,8,8a-hexahydropyrido[4,3-b][1,4]oxazin-3-one; or
(4aR,8aS)-6-[3-[[4-fluoro-2-(trifluoromethyl)phenyl]methoxy]azetidine-1-carbonyl]-4,4a,5,7,8,8a-hexahydropyrido[4,3-b][1,4]oxazin-3-one,
or a pharmaceutically acceptable salt thereof.

58. A method of manufacturing a compound of claim 42, or pharmaceutically acceptable salt thereof, the method comprising:
(a) reacting a first amine 4a,5,6,7,8,8a-hexahydro-4H-pyrido[4,3-b][1,4]oxazin-3-one (1), 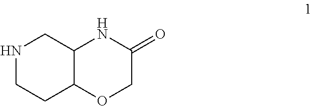 [SMILES: O=C1COC2CCNCC2N1]
with a second amine 2, wherein A, L¹, m, n, and R¹ to R⁴ are as defined in claim 42, 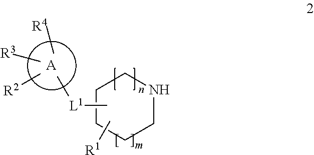
in the presence of a base and a urea forming reagent, to form a compound of formula (IA), wherein A, L¹, m, n, and R¹ to R⁴ are as defined in claim 42, 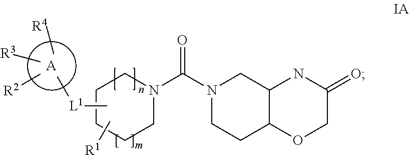
or
(b) reacting 4a,5,6,7,8,8a-hexahydro-4H-pyrido[4,3-b][1,4]oxazin-3-one(1), 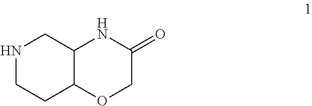 [SMILES: O=C1COC2CCNCC2N1]
with a carboxylic acid 3a, wherein A, L¹, m, n, and R¹ to R⁴ are as defined in claim 42, 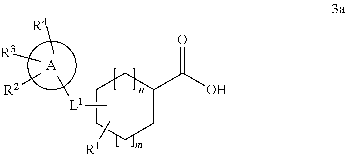
in the presence of a coupling reagent and optionally a base to form a compound of formula (IB), wherein A, L¹, m, n, and R¹ to R⁴ are as defined in claim 42, 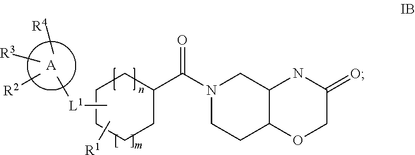
or
(c) reacting 4a,5,6,7,8,8a-hexahydro-4H-pyrido[4,3-b][1,4]oxazin-3-one(1), 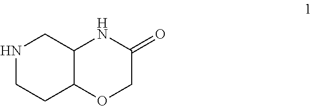 [SMILES: O=C1COC2CCNCC2N1]
with a carboxylic acid chloride 3b, wherein A, L¹, m, n, and R¹ to R⁴ are as defined in claim 42, 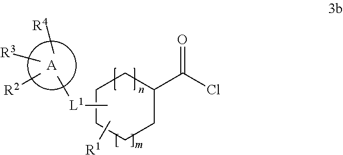
in the presence of a base, to form a compound of formula (IB), wherein A, L¹, m, n, and R¹ to R⁴ are as defined in claim 42,

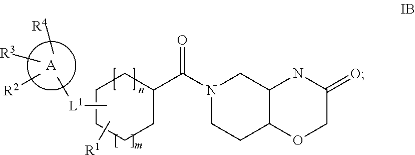

59. A compound of claim 42, or a pharmaceutically acceptable salt thereof, when manufactured according to a method comprising:
(a) reacting a first amine 4a,5,6,7,8,8a-hexahydro-4H-pyrido[4,3-b][1,4]oxazin-3-one (1), 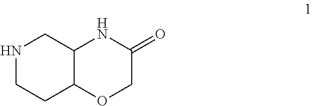 [SMILES: O=C1COC2CCNCC2N1]
with a second amine 2, wherein A, L¹, m, n, and R¹ to R⁴ are as defined in claim 42, 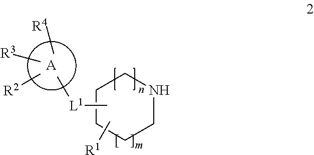
in the presence of a base and a urea forming reagent, to form a compound of formula (IA), wherein A, L¹, m, n, and R¹ to R⁴ are as defined in claim 42, 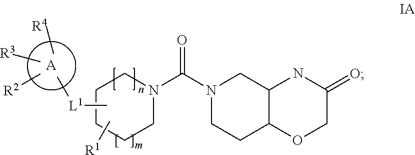
or
(b) reacting 4a,5,6,7,8,8a-hexahydro-4H-pyrido[4,3-b][1,4]oxazin-3-one(1), 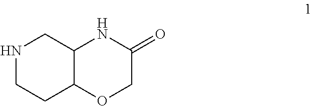 [SMILES: O=C1COC2CCNCC2N1]
with a carboxylic acid 3a, wherein A, L¹, m, n, and R¹ to R⁴ are as defined in claim 42, 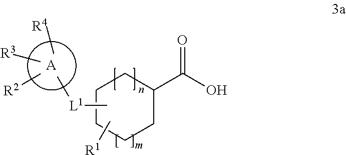
in the presence of a coupling reagent and optionally a base to form a compound of formula (IB), wherein A, L¹, m, n, and R¹ to R⁴ are as defined in claim 42, 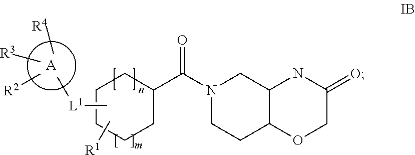
or
(c) reacting 4a,5,6,7,8,8a-hexahydro-4H-pyrido[4,3-b][1,4]oxazin-3-one (1), 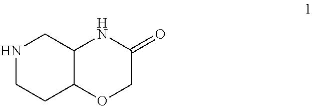 [SMILES: O=C1COC2CCNCC2N1]
with a carboxylic acid chloride 3b, wherein A, L¹, m, n, and R¹ to R⁴ are as defined in claim 42, 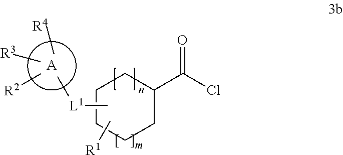
in the presence of a base, to form a compound of formula (IB), wherein A, L¹, m, n, and R¹ to R⁴ are as defined in claim 42,

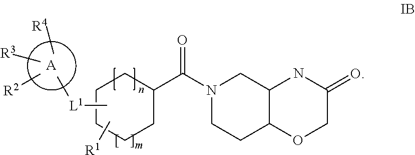

60. A pharmaceutical composition comprising a compound of claim 42, or a pharmaceutically acceptable salt thereof, and a therapeutically inert carrier.

61. A pharmaceutical composition comprising a compound of claim 57, or a pharmaceutically acceptable salt thereof, and a therapeutically inert carrier.

62. A method for the treatment or prophylaxis of a disease or disorder in a mammal, the method comprising administering an effective amount of a compound of claim 42, or a pharmaceutically acceptable salt thereof, to the mammal,
wherein the disease or disorder is neuroinflammation, neurodegenerative disease, pain, cancer, mental disorder, or inflammatory bowel disease.

63. The method of claim 62, wherein the disease or disorder is multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain in a mammal, abdominal pain, abdominal pain associated with irritable bowel syndrome, or visceral pain.
